(19) United States
(12) Patent Application Publication
(10) Pub. No.: US 2022/0267449 A1
(43) Pub. Date: Aug. 25, 2022

(54) METHODS OF PRODUCING AN ANTI-a4B7 ANTIBODY

(71) Applicant: Takeda Pharmaceutical Company Limited, Osaka (JP)

(72) Inventors: Debra Ameli, NW Oak Grove, MN (US); Susan R. Carter, Champlin, MN (US); Michael E. Dolan, Watertown, MA (US); Nicole Hilo, Minneapolis, MN (US); Amitava Kundu, Maple Grove, MN (US); Amy Miller, Ham Lake, MN (US); George Parks, Wakefield, MA (US); Olga Paley, Somerville, MA (US); Paras Bhatia, Lowell, MA (US)

(21) Appl. No.: 17/596,421

(22) PCT Filed: Jun. 10, 2020
(86) PCT No.: PCT/US2020/037059

§ 371 Date: Dec. 9, 2021

Related U.S. Application Data
(60) Provisional application No. 62/859,494, filed on Jun. 10, 2019.

(51) Int. Cl. C07K 16/28; C07K 16/30

(52) U.S. Cl. CPC C07K 16/2839; C07K 2317/24; C07K 2317/14; C07K 16/30

(57) ABSTRACT

Provided herein are methods for purifying an anti-α4β7 integrin antibody, such as vedolizumab, from a liquid solution, e.g., from a mammalian cell culture clarified harvest. The invention relates, inter alia, to purification methods for controlling the amount of product-related substances and/or process-related impurities present in purified preparations of an anti-α4β7 integrin antibody, or antigen-binding fragment thereof, e.g., vedolizumab. Compositions comprising an anti-α4β7 antibody, and uses thereof to treat a disorder, are also provided.

### RELATED APPLICATIONS

This application is a U.S. National Phase Application under 35 U.S.C. § 371 of International Application No. PCT/US2020/037059, filed on Jun. 10, 2020, which claims priority to U.S. Provisional Application 62/859,494 filed on Jun. 10, 2019. The entire content of each of the foregoing applications is incorporated herein by reference.

### FIELD OF THE INVENTION

The present invention relates to methods for purifying an anti-α4β7 antibody, or a fragment thereof.

### SEQUENCE LISTING

The instant application contains a Sequence Listing which has been submitted electronically in ASCII format and is hereby incorporated by reference in its entirety. Said ASCII copy, created on Jun. 5, 2020, is named T103022_1090US_SL.txt and is 10,009 bytes in size. The entire contents of the Sequence Listing in the sequence listing.txt file are incorporated herein.

### BACKGROUND

Large-scale, economic purification of proteins is an increasingly important concern in the biotechnology industry. Generally, biologic medicines are produced by cell culture using prokaryotic, e.g., bacterial, or eukaryotic, e.g., mammalian or fungal, cell lines that have been engineered to produce the therapeutic protein of interest in large quantities. Since the cell lines used are living organisms, they must be fed a complex cell culture medium comprising sugars, amino acids, and growth factors, sometimes supplied from preparations of animal serum. Separation of the desired recombinant therapeutic protein from process-related impurities, including, for example, cell culture media components, host cell proteins (HCPs), host nucleic acids, and/or chromatographic materials, as well as product-related impurities such as aggregates, mis-folded species, or fragments of the protein of interest, to a purity sufficient for use as a human therapeutic poses a formidable challenge.

Product-related and process-related impurities, including aggregates, have the potential to interfere with the purification process, affect the protein during storage, and/or can potentially be a cause of adverse reactions upon administration of the antibody to a subject (Shukla et al., J. Chromatogr. B. Analyt. Technol. Biomed. Life Sci., 848(1), 28-39).

Accordingly, there remains a need in the art for improved methods of purification of therapeutic proteins, e.g., antibodies, while removing impurities effectively, improving the recovery rate of the protein, and maintaining therapeutic requirements.

### SUMMARY OF THE INVENTION

The present invention is based, at least in part, on the development of processes for the production of an anti-α4β7 antibody, or an antigen binding portion thereof. In some embodiments, the anti-α4β7 antibody, or an antigen binding portion thereof, is a humanized anti-α4β7 antibody, or an antigen binding portion thereof. In any of the following aspects and embodiments, the humanized anti-α4β7 antibody, or an antigen binding portion thereof can comprise a heavy chain variable region comprising a CDR1 set forth in SEQ ID NO:2, a CDR2 set forth in SEQ ID NO:3, and a CDR3 set forth in SEQ ID NO:4, and/or a light chain variable region comprising a CDR1 set forth in SEQ ID NO:6, a CDR2 set forth in SEQ ID NO:7, and a CDR3 set forth in SEQ ID NO:8. In some embodiments, the humanized anti-α4β7 antibody, or an antigen binding portion thereof comprises a heavy chain variable region comprising SEQ ID NO:1, and/or a light chain variable region comprising SEQ ID NO:2. In some embodiments, the humanized anti-α4β7 antibody, or an antigen binding portion thereof comprises a heavy chain comprising SEQ ID NO:9, and/or a light chain comprising SEQ ID NO:10. In exemplary embodiments, the anti-α4β7 antibody is vedolizumab, or an antigen binding portion thereof.

Accordingly, in one aspect, the invention provides a method of producing a composition comprising a humanized anti-α4β7 antibody, or an antigen binding portion thereof, comprising: providing a composition comprising the humanized anti-α4β7 antibody, or an antigen binding portion thereof at a pH greater than pH 6.5; and incubating the composition comprising the humanized anti-α4β7 antibody, or an antigen binding portion thereof for a period of at least 20 minutes to 10 hours; thereby producing a composition comprising the humanized anti-α4β7 antibody, or an antigen binding portion thereof.

In another aspect, the invention provides a method of producing a composition comprising a humanized anti-α4β7 antibody, or an antigen binding portion thereof having a reduced level of basic isoform species, comprising: providing a composition comprising the humanized anti-α4β7 antibody, or an antigen binding portion thereof at a pH greater than pH 6.5; and incubating the composition comprising the humanized anti-α4β7 antibody, or an antigen binding portion thereof for a period of time sufficient to reduce the level of basic isoform species of the anti-α4β7 antibody, or an antigen binding portion thereof in the composition; thereby producing a composition comprising the humanized anti-α4β7 antibody, or an antigen binding portion thereof having a reduced level of basic isoform species.

In some embodiments of the foregoing aspects, the humanized anti-α4β7 antibody, or antigen binding portion thereof comprises a heavy chain variable region comprising a CDR1 set forth in SEQ ID NO:2, a CDR2 set forth in SEQ ID NO:3, and a CDR3 set forth in SEQ ID NO:4, and/or a light chain variable region comprising a CDR1 set forth in SEQ ID NO:6, a CDR2 set forth in SEQ ID NO:7, and a CDR3 set forth in SEQ ID NO:8. In some embodiments, the humanized anti-α4β7 antibody, or antigen binding portion thereof comprises a heavy chain variable region comprising SEQ ID NO:1, and/or a light chain variable region comprising SEQ ID NO:2. In some embodiments, the humanized anti-α4β7 antibody, or antigen binding portion thereof comprises a heavy chain comprising SEQ ID NO:9, and/or a light chain comprising SEQ ID NO:10. In exemplary embodiments, the anti-α4β7 antibody is vedolizumab, or an antigen binding portion thereof.

In some embodiments, the method produces a composition comprising the humanized anti-α4β7 antibody, or antigen binding portion thereof having <16%, <15%, <14%, <13%, <12%, <11% or <10% basic isoform species.

In some embodiments, the incubation is performed during purification of the humanized anti-α4β7 antibody, or antigen binding portion thereof, and wherein the incubation is performed (a) prior to ultrafiltration/diafiltration (UF/DF) of the antibody, or (b) prior to formulation of the antibody in a pharmaceutically acceptable buffer.

In some embodiments, the incubation is performed at ambient temperature. In some embodiments, the incubation is performed at 15-30° C. In some embodiments, the incubation is performed at 20-25° C.

In some embodiments, the composition comprising the humanized anti-α4β7 antibody, or antigen binding portion thereof is provided at a pH of about 6.5-8.5. In some embodiments, the composition comprising the humanized anti-α4β7 antibody, or antigen binding portion thereof is provided at a pH of about 7.0-8.0. In some embodiments, the composition comprising the humanized anti-α4β7 antibody, or antigen binding portion thereof is provided at a pH of about 7.0-7.5. In other embodiments, the composition comprising the humanized anti-α4β7 antibody, or antigen binding portion thereof is provided at a pH of about 6.6-7.3. In some embodiments, the composition comprising the humanized anti-α4β7 antibody, or antigen binding portion thereof is provided at a pH of about pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, or pH 8.5.

In some embodiments, the composition comprising the humanized anti-α4β7 antibody, or antigen binding portion thereof is incubated for a period of about 10-120 hours. In some embodiments, the composition comprising the humanized anti-α4β7 antibody, or antigen binding portion thereof is incubated for a period of about 12-120 hours. In some embodiments, the composition comprising the humanized anti-α4β7 antibody, or antigen binding portion thereof is incubated for a period of about 12-96 hours. In some embodiments, the composition comprising the humanized anti-α4β7 antibody, or antigen binding portion thereof is incubated for a period of about 12-72 hours. In some embodiments, the composition comprising the humanized anti-α4β7 antibody, or antigen binding portion thereof is incubated for a period of about 12-48 hours. In some embodiments, the composition comprising the humanized anti-α4β7 antibody, or antigen binding portion thereof is incubated for a period of at least 12 hours. In some embodiments, the composition comprising the humanized anti-α4β7 antibody, or antigen binding portion thereof is incubated for a period of at least 15-36 hours. In some embodiments, the composition comprising the humanized anti-α4β7 antibody, or antigen binding portion thereof is incubated for a period of about 24-120 hours. In some embodiments, the composition comprising the humanized anti-α4β7 antibody, or antigen binding portion thereof is incubated for a period of about 24-96 hours. In some embodiments, the composition comprising the humanized anti-α4β7 antibody, or antigen binding portion thereof is incubated for a period of about 24-72 hours. In some embodiments, the composition comprising the humanized anti-α4β7 antibody, or antigen binding portion thereof is incubated for a period of about 24-48 hours. In some embodiments, the composition comprising the humanized anti-α4β7 antibody, or antigen binding portion thereof is incubated for a period of about 10 hours, about 12 hours, about 24 hours, about 36 hours, about 48 hours, about 72 hours, about 96 hours, or about 120 hours.

In another aspect, provided herein is a method of purifying a humanized anti-α4β7 antibody or an antigen binding portion thereof from a clarified cell culture harvest comprising (i) providing a clarified cell culture harvest obtained from a culture of recombinant host cells expressing the anti-α4β7 antibody or an antigen binding portion thereof, and (ii) purifying the anti-α4β7 antibody or an antigen binding portion thereof from the cell culture harvest, wherein the antibody is exposed to a pH at or below 4.0 for no more than 24 hours, wherein the anti-α4β7 antibody or antigen binding portion thereof comprises a heavy chain variable region comprising the amino acid sequence set forth in SEQ ID NO:1, and a light chain variable region comprising the amino acid sequence set forth in SEQ ID NO:5.

In some embodiments, the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 4.0 (e.g., pH 3.6-4.0) for a longer duration of time, i.e., greater than 24 hours.

In some embodiments, the anti-α4β7 antibody, or an antigen binding portion thereof is vedolizumab, or an antigen binding portion thereof.

In some embodiments, the composition comprising the humanized anti-α4β7 antibody, or antigen binding portion thereof, e.g., vedolizumab, or an antigen binding portion thereof, comprises a first basic isoform peak (BP1) and a second basic isoform peak (BP2), and wherein the method produces a composition comprising the humanized anti-α4β7 antibody, or antigen binding portion thereof, having a reduced level of BP2. In certain embodiments, the method produces a composition comprising the humanized anti-α4β7 antibody, or antigen binding portion thereof, having less than 2%, less than 1.5%, less than 1%, or less than 0.7% BP2.

In some embodiments, the composition comprising the humanized anti-α4β7 antibody, or antigen binding portion thereof is derived from a mammalian cell culture expressing the humanized anti-α4β7 antibody, or antigen binding portion thereof. In certain embodiments, the mammalian cell culture is a Chinese Hamster Ovary (CHO) cell culture. In certain embodiments, the CHO cell culture comprises CHO cells that lack dihydrofolate reductase (DHFR) expression. In certain embodiments, the CHO cell culture comprises CHO cells that lack glutamine synthetase (GS) expression.

In some embodiments, the method further comprises purifying the composition comprising the humanized anti-α4β7 antibody, or antigen binding portion thereof from mammalian host cell protein (HCP) using one or more chromatographic separation steps selected from the group consisting of affinity chromatography, cation exchange chromatography, anion exchange chromatography, mixed mode chromatography, ceramic hydroxyapatite (CHT) chromatography, and hydrophobic interaction chromatography (HIC).

In certain embodiments, the composition comprising the humanized anti-α4β7 antibody, or antigen binding portion thereof is purified using an affinity chromatography resin comprising Protein A.

In certain embodiments, the composition comprising the humanized anti-α4β7 antibody, or antigen binding portion thereof is purified using affinity chromatography prior to the incubation to reduce basic isoforms. In certain embodiments, the composition comprising the humanized anti-α4β7 antibody, or antigen binding portion thereof is purified using affinity chromatography following the incubation.

In certain embodiments, the composition comprising the humanized anti-α4β7 antibody, or antigen binding portion thereof is purified using cation exchange chromatography. In certain embodiments, the composition comprising the humanized anti-α4β7 antibody, or antigen binding portion thereof is purified using cation exchange chromatography prior to the incubation. In certain embodiments, the composition comprising the humanized anti-α4β7 antibody, or antigen binding portion thereof is purified using cation exchange chromatography following the incubation.

In certain embodiments, the composition comprising the humanized anti-α4β7 antibody, or antigen binding portion thereof is purified using anion exchange chromatography. In certain embodiments, the composition comprising the humanized anti-α4β7 antibody, or antigen binding portion thereof is purified using anion exchange chromatography prior to the incubation. In certain embodiments, the composition comprising the humanized anti-α4β7 antibody, or antigen binding portion thereof is purified using anion exchange chromatography following the incubation.

In certain embodiments, the composition comprising the humanized anti-α4β7 antibody, or antigen binding portion thereof is purified using CHT chromatography. In certain embodiments, the composition comprising the humanized anti-α4β7 antibody, or antigen binding portion thereof is purified using CHT chromatography prior to the incubation. In certain embodiments, the composition comprising the humanized anti-α4β7 antibody, or antigen binding portion thereof is purified using CHT chromatography following the incubation.

In some embodiments, the method comprises incorporating the composition into a pharmaceutical formulation.

In certain embodiments, the pharmaceutical formulation is a lyophilized pharmaceutical formulation. In particular embodiments, the lyophilized pharmaceutical formulation is a dry, lyophilized pharmaceutical formulation. In some such embodiments, the method further comprises a step of reconstituting the dry, lyophilized pharmaceutical formulation with a liquid so that it is suitable for administration.

In alternative embodiments, the pharmaceutical formulation is a liquid pharmaceutical formulation. In some such embodiments, the liquid pharmaceutical formulation is suitable for subcutaneous administration to a human.

In another embodiment, the invention provides a method of purifying a humanized anti-α4β7 antibody, or antigen binding portion thereof with a reduced level of basic isoform species, wherein the humanized anti-α4β7 antibody, or antigen binding portion thereof is maintained at or above pH 6.5 for at least 60%, at least 65%, at least 70%, at least 75%, at least 80%, at least 85%, at least 90%, at least 95%, at least 97%, at least 98%, or at least 99% of the total time between primary recovery of the humanized anti-α4β7 antibody, or antigen binding portion thereof from a cell culture harvest to formulation of the humanized anti-α4β7 antibody, or antigen binding portion thereof in a pharmaceutically acceptable carrier by ultrafiltration/diafiltration (UF/DF). By maintaining the humanized anti-α4β7 antibody, or antigen binding portion thereof at a pH at or above pH 6.5 for the majority of the purification process, basic isoform species of the humanized anti-α4β7 antibody, or antigen binding portion thereof can be reduced, relative to an equivalent purification process in which the humanized anti-α4β7 antibody, or antigen binding portion thereof is at a pH below pH 6.5 for a significant time, for example, greater than 5%, greater than 10%, greater than 15%, greater than 20%, greater than 25%, greater than 30%, greater than 35%, or greater than 40% of the total time between primary recovery of the humanized anti-α4β7 antibody, or antigen binding portion thereof from a cell culture harvest to formulation of the humanized anti-α4β7 antibody, or antigen binding portion thereof in a pharmaceutically acceptable carrier by ultrafiltration/diafiltration (UF/DF).

In another aspect, the invention provides a composition comprising a humanized anti-α4β7 antibody, or antigen binding portion thereof (e.g., vedolizumab) having a reduced level of basic isoform species. In some embodiments, the basic isoform species of the humanized anti-α4β7 antibody, or antigen binding portion thereof comprises less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of the humanized anti-α4β7 antibody, or antigen binding portion thereof present in the composition. In some embodiments, the composition comprising the humanized anti-α4β7 antibody, or antigen binding portion thereof, having a reduced level of basic isoform species is produced using the methods described herein. In some embodiments, the composition comprising the humanized anti-α4β7 antibody, or antigen binding portion thereof is produced by a combination of the methods of the invention.

In another aspect, provided herein is a method of producing a composition comprising a humanized anti-α4β7 antibody, or antigen binding portion thereof (e.g., vedolizumab), comprising: (a) contacting a sample containing the humanized anti-α4β7 antibody, or antigen binding portion thereof and host cell protein (HCP) with an anion exchange resin in the presence of a loading buffer, wherein the loading buffer has a conductivity of 11 mS/cm or less, such that HCP binds to the anion exchange resin; and (b) collecting the flow through material from the anion exchange resin, wherein the flow through material comprises the humanized anti-α4β7 antibody, or antigen binding portion thereof.

In another aspect, the invention provides a method of producing a composition comprising a humanized anti-α4β7 antibody, or antigen binding portion thereof (e.g., vedolizumab), comprising: (a) contacting a sample containing the humanized anti-α4β7 antibody, or antigen binding portion thereof and host cell protein (HCP) with an anion exchange resin in the presence of a loading buffer, wherein the loading buffer has a conductivity of 11 mS/cm or less (e.g., about 11 mS/cm or less, about 10 mS/cm or less, about 9 mS/cm or less, about 8 mS/cm or less, about 7 mS/cm or less, about 6 mS/cm or less, about 5 mS/cm or less, about 4 mS/cm or less, about 3 mS/cm or less, or about 2 mS/cm or less), such that HCP binds to the anion exchange resin; (b) contacting the anion exchange resin with an elution buffer; and (c) collecting the flow through from the anion exchange resin, wherein the flow through comprises the humanized anti-α4β7 antibody, or antigen binding portion thereof.

In some embodiments of the above aspects, the method is a method of producing a composition comprising the humanized anti-α4β7 antibody, or antigen binding portion thereof, e.g., vedolizumab, having a reduced amount of HCP, and the flow through comprises the humanized anti-α4β7 antibody, or antigen binding portion thereof, e.g., vedolizumab, and a reduced amount of HCP.

In some embodiments, the loading buffer has a conductivity of 9 mS/cm to 11 mS/cm (e.g., about 9 mS/cm to about 11 mS/cm, or about 10 mS/cm to about 11 mS/cm).

In some embodiments, the loading buffer has a conductivity of less than 11 mS/cm (e.g., less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, or less than 2 mS/cm).

In some embodiments, the loading buffer has a conductivity of about 9 mS/cm, 9.5 mS/cm, 10 mS/cm, 10.5 mS/cm, or 11 mS/cm.

In some embodiments, the HCP is a Chinese Hamster Ovary (CHO) cell protein. In certain embodiments, the HCP is derived from a CHO cell that lacks dihydrofolate reductase (DHFR) expression. In certain embodiments, the HCP is derived from a CHO cell that lacks glutamine synthetase (GS) expression.

In some embodiments, the anion exchange resin is washed with a wash buffer. In some embodiments, the wash buffer has a conductivity of 11 mS/cm or less. In some embodiments, the wash buffer has a conductivity of 9 mS/cm to 11 mS/cm. In some embodiments, the wash buffer has a conductivity of less than 11 mS/cm. In some embodiments, the wash buffer has a conductivity of about 9 mS/cm, 9.5 mS/cm, 10 mS/cm, 10.5 mS/cm, or 11 mS/cm. In some embodiments, the wash buffer has the same conductivity as the loading buffer.

In some embodiments, the loading buffer comprises sodium chloride and/or sodium phosphate.

In some embodiments, the loading buffer comprises 20-150 mM salt, 50-125 mM salt, or 75-100 mM salt. In one embodiment, the salt comprises sodium chloride and/or sodium phosphate. For example, the buffer can comprise about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, or about 150 mM sodium chloride. In addition, or in the alternative, the buffer can comprise about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, or about 150 mM sodium phosphate.

In some embodiments, the wash buffer comprises sodium chloride and/or sodium phosphate.

In some embodiments, the wash buffer comprises 20-150 mM salt, 50-125 mM salt, or 75-100 mM salt. In one embodiment, the salt comprises sodium chloride and/or sodium phosphate. For example, the buffer can comprise about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, or about 150 mM sodium chloride. In addition, or in the alternative, the buffer can comprise about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, or about 150 mM sodium phosphate.

In some embodiments, the wash buffer has a conductivity at or below that of the loading buffer. In some embodiments, the wash buffer has the same conductivity as the loading buffer.

In some embodiments, the anion exchange resin is formatted as an anion exchange column or an anion exchange membrane.

In some embodiments, the anion exchange resin comprises a quaternary amine functional group.

In some embodiments, the sample containing the humanized anti-α4β7 antibody, or antigen binding portion thereof (e.g., vedolizumab) and HCP is derived from a mammalian cell culture following one or more chromatographic separation steps. In certain embodiments, the one or more chromatographic separation steps comprise one or more steps selected from the group consisting of affinity chromatography, cation exchange chromatography, mixed mode chromatography, hydrophobic interaction chromatography (HIC) and ceramic hydroxyapatite (CHT) chromatography.

In some embodiments, the amount of HCP in the flow through is 8 ppm or less, 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, or 2 ppm or less.

In some embodiments, the amount of HCP in the flow through is reduced by at least 50% (e.g., at least about 50%, at least about 55%, at least about 60%, at least about 65%, at least about 70%, at least about 75%, at least about 80%, at least about 85%, at least about 90%, at least about 95%, or at least about 98% or more than about 98%) relative to the amount of HCP in a flow through produced when the method is performed using the same sample with a loading buffer having a conductivity greater than 12 mS/cm.

In some embodiments, the method further comprises processing the flow through to exchange the buffer by a process comprising ultrafiltration and/or diafiltration to a buffer comprising one or more pharmaceutically acceptable carriers or excipients.

In some embodiments, the method comprises incorporating the composition into a pharmaceutical formulation.

In certain embodiments, the pharmaceutical formulation is a lyophilized pharmaceutical formulation. In particular embodiments, the lyophilized pharmaceutical formulation is a dry, lyophilized pharmaceutical formulation. In some such embodiments, the method further comprises a step of reconstituting the dry, lyophilized pharmaceutical formulation with a liquid so that it is suitable for administration.

In alternative embodiments, the pharmaceutical formulation is a liquid pharmaceutical formulation. In some such embodiments, the liquid pharmaceutical formulation is suitable for subcutaneous administration to a human.

In another aspect, provided herein is a composition comprising a humanized anti-α4β7 antibody, or antigen binding portion thereof (e.g., vedolizumab) produced by any method of the invention. Also provided herein is a composition comprising a humanized anti-α4β7 antibody, or antigen binding portion thereof (e.g., vedolizumab), wherein the composition is obtainable by any method of the invention. In some embodiments, the amount of HCP in the composition is 8 ppm or less, 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, or 2 ppm or less. In another embodiment, the basic isoform species of the humanized anti-α4β7 antibody, or antigen binding portion thereof (e.g., vedolizumab) comprises less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of the antibody species present in the composition.

In some embodiments, the composition comprising the humanized anti-α4β7 antibody, or antigen binding portion thereof (e.g., vedolizumab) is produced by a combination of methods of the invention.

In another aspect, the invention is directed to a method of increasing the yield of a humanized anti-α4β7 antibody, or antigen binding portion thereof (e.g., vedolizumab) recovered following elution from a mixed mode chromatography resin, comprising equilibrating the mixed mode chromatography resin with an equilibration buffer, loading a solution comprising the anti-α4β7 antibody, or antigen binding portion thereof and a loading buffer onto the mixed mode chromatography resin such that the anti-α4β7 antibody, or antigen binding portion thereof binds the mixed mode chromatography resin, washing the mixed mode chromatography resin with a wash buffer, and eluting the anti-α4β7 antibody, or antigen binding portion thereof from the mixed mode chromatography resin with an elution buffer, wherein the equilibration buffer, the loading buffer, and/or the wash buffer have a pH at or below 7.0.

In one embodiment, the equilibration buffer, the loading buffer, and/or the wash buffer have a pH of 6.0-7.0. In another embodiment, the equilibration buffer, the loading buffer, and/or the wash buffer have a pH of 6.5-7.0. In another embodiment, the equilibration buffer, the loading buffer, and/or the wash buffer have a pH of 6.6-6.8.

In another embodiment, the equilibration buffer, the loading buffer, and/or the wash buffer have a salt concentration of 30 mM to 70 mM. In another embodiment, the equilibration buffer, the loading buffer, and/or the wash buffer have a salt concentration of 40 mM to 70 mM. In another embodiment, the equilibration buffer, the loading buffer, and/or the wash buffer have a salt concentration of 50 mM to 65 mM. In another embodiment, the loading buffer, and/or the wash buffer have a salt concentration of 55 mM-65 mM. In another embodiment, the salt comprises sodium chloride and/or sodium phosphate.

In another embodiment, the equilibration buffer, the loading buffer, and/or the wash buffer have a sodium chloride concentration of 30 mM to 70 mM. In another embodiment, the loading buffer, and/or the wash buffer have a sodium chloride concentration of 40 mM to 70 mM. In another embodiment, the equilibration buffer, the loading buffer, and/or the wash buffer have a sodium chloride concentration of 40 mM to 60 mM. In another embodiment, the equilibration buffer, the loading buffer, and/or the wash buffer have a sodium chloride concentration of 45 mM-55 mM. In another embodiment, the equilibration buffer, the loading buffer, and/or the wash buffer have a sodium chloride concentration of about 50 mM. In another embodiment, the equilibration buffer, the loading buffer, and/or the wash buffer further comprise sodium phosphate.

In some embodiments, the equilibration buffer, the loading buffer, and/or the wash buffer have the same pH. In some embodiments, the equilibration buffer, the loading buffer, and/or the wash buffer have the same salt concentration. In some embodiments, the equilibration buffer, the loading buffer, and/or the wash buffer can be the same buffer. In some embodiments of the foregoing aspect, the mixed mode resin can be a ceramic hydroxyapatite resin, e.g., a CHT resin.

In another aspect, provided herein is a low basic species composition comprising an anti-α4β7 antibody, wherein the composition comprises less than 16%, less than 15%, less than 14%, less than 13%, less than 12%, less than 11%, or less than 10% total basic isoform species of the anti-α4β7 antibody, wherein the basic isoform species have a net positive charge relative to a main isoform of the anti-α4β7 antibody, and wherein the anti-α4β7 antibody comprises a heavy chain variable region comprising SEQ ID NO:1, and a light chain variable region comprising SEQ ID NO:2. In some embodiments, basic isoform species can be quantified by cation exchange (CEX) chromatography. For example, in some embodiments, basic isoform species can be quantified by determining the relative area of peaks that elute more slowly from a cation exchange (CEX) resin than a peak corresponding to the main isoform.

Accordingly, in another aspect, provided herein is a low basic species composition comprising an anti-α4β7 antibody, wherein the composition comprises less than 16%, less than 15%, less than 14%, less than 13%, less than 12%, less than 11%, or less than 10% total basic isoform species of the anti-α4β7 antibody, wherein the basic isoform species have a net positive charge relative to a main isoform of the anti-α4β7 antibody and can be quantified by determining the relative area of peaks that elute more slowly from a cation exchange (CEX) resin than a peak corresponding to the main isoform, and wherein the anti-α4β7 antibody comprises a heavy chain variable region comprising SEQ ID NO:1, and a light chain variable region comprising SEQ ID NO:2.

In some embodiments, the composition comprises a first basic isoform peak (BP1) and a second basic isoform peak (BP2). In some embodiments, the composition comprises less than 2% BP2. In some embodiments, the composition comprises less than 1.5% BP2. In some embodiments, the composition comprises less than 1% BP2. In some embodiments, the composition comprises less than 0.7% BP2.

In some embodiments, the ratio of BP1 to BP2 is at least 3. In some embodiments, the ratio of BP1 to BP2 is at least 5. In some embodiments, the ratio of BP1 to BP2 is at least 7. In some embodiments, the ratio of BP1 to BP2 is at least 10.

In some embodiments, the composition comprises less than 8% total basic isoform species of the anti-α4β7 antibody. In some embodiments, the composition comprises less than 7% total basic isoform species of the anti-α4β7 antibody. In some embodiments, the composition comprises less than 6% total basic isoform species of the anti-α4β7 antibody. In some embodiments, the composition comprises less than 5% total basic isoform species of the anti-α4β7 antibody.

In another aspect, provided herein is a pharmaceutical composition comprising a composition provided herein and a pharmaceutically acceptable carrier or excipient. In some embodiments, the pH of the pharmaceutical composition is between 6.0-7.0. In some embodiments, the pH of the pharmaceutical composition is about pH 6.3. In other embodiments, the pH of the pharmaceutical composition is pH 6.3 to pH 6.5.

In some embodiments, the pharmaceutical composition further comprises an amino acid. In some embodiments, the amino acid is arginine or histidine.

In some embodiments, the pharmaceutical composition further comprises a sugar. In certain embodiments, the sugar is sucrose or trehalose.

In some embodiments, the pharmaceutical composition comprises arginine, histidine, and sucrose. In some embodiments, the pharmaceutical composition comprises arginine, histidine, sucrose and polysorbate 80.

In some embodiments, the pharmaceutical composition comprises at least 200 mg, at least 250 mg, or at least 300 mg of the anti-α4β7 antibody. In some embodiments, the pharmaceutical composition comprises about 300 mg of the anti-α4β7 antibody.

In some embodiments, the anti-α4β7 antibody is vedolizumab, or an antigen binding portion thereof.

In another aspect, provided herein is a method of producing a low basic species composition comprising an anti-α4β7 antibody having less than 16%, less than 15%, less than 14%, less than 13%, less than 12%, less than 11%, or less than 10% total basic isoform species, the method comprising providing a composition comprising the anti-α4β7 antibody at a pH greater than pH 6.3; and incubating the composition comprising the anti-α4β7 antibody for a period of greater than 10 hours; thereby producing a low basic species composition comprising the anti-α4β7 antibody having less than 16%, less than 15%, less than 14%, less than 13%, less than 12%, less than 11%, or less than 10% total basic isoform species. In some embodiments, the anti-α4β7 antibody comprises a heavy chain variable region comprising SEQ ID NO:1, and a light chain variable region comprising SEQ ID NO:2.

In another aspect, provided herein is a method of producing a low basic species composition comprising an anti-α4β7 antibody having less than 16%, less than 15%, less than 14%, less than 13%, less than 12%, less than 11%, or less than 10% total basic isoform species, the method comprising providing a composition comprising vedolizumab at a pH greater than pH 6.3; and incubating the composition comprising vedolizumab for a period of time sufficient to reduce the level of basic vedolizumab isoform species in the composition; thereby producing a low basic species composition comprising the anti-α4β7 antibody having less than 16%, less than 15%, less than 14%, less than 13%, less than 12%, less than 11%, or less than 10% total basic isoform species. In some embodiments, the anti-α4β7 antibody comprises a heavy chain variable region comprising SEQ ID NO:1, and a light chain variable region comprising SEQ ID NO:2.

In general, basic isoform species have a net positive charge relative to a main isoform of the anti-α4β7 antibody. In some embodiments, the level of basic isoform species can be quantified using cation exchange chromatography (CEX). In some embodiments, the level of basic isoform species can be quantified by determining the relative area of peaks that elute more slowly from a cation exchange (CEX) resin than a peak corresponding to the main isoform. In some embodiments, the composition comprises a first basic isoform peak (BP1) and a second basic isoform peak (BP2). In some embodiments, the composition comprises less than 2% BP2. In some embodiments, the composition comprises less than 1.5% BP2. In some embodiments, the composition comprises less than 1% BP2. In some embodiments, the composition comprises less than 0.7% BP2.

In some embodiments, the ratio of BP1 to BP2 is at least 3. In some embodiments, the ratio of BP1 to BP2 is at least 5. In some embodiments, the ratio of BP1 to BP2 is at least 7. In some embodiments, the ratio of BP1 to BP2 is at least 10.

In some embodiments, the composition comprises less than 8% total basic isoform species of the anti-α4β7 antibody. In some embodiments, the composition comprises less than 7% total basic isoform species of the anti-α4β7 antibody. In some embodiments, the composition comprises less than 6% total basic isoform species of the anti-α4β7 antibody. In some embodiments, the composition comprises less than 5% total basic isoform species of the anti-α4β7 antibody.

In some embodiments, provided herein is a composition comprising an anti-α4β7 antibody that is obtainable by the foregoing methods. In some embodiments, the antibody is vedolizumab, or an antigen binding portion thereof.

In some embodiments, provided herein is a composition comprising an anti-α4β7 antibody that is obtained by the foregoing methods. In some embodiments, the antibody is vedolizumab, or an antigen binding portion thereof.

In another aspect, provided herein is a method for treating a disease or disorder in a human subject, wherein the method comprises administering to a subject a pharmaceutical composition provided herein comprising an anti-α4β7 antibody (e.g., vedolizumab) in an amount effective to treat the disease or disorder in the human subject. In some embodiments, the pharmaceutical composition comprises a composition of vedolizumab having a reduced level of basic vedolizumab isoform species, and/or having a reduced level of host cell protein, as provided herein, and/or as produced in accordance with the methods provided herein. In some embodiments, the anti-α4β7 antibody comprises a heavy chain variable region comprising SEQ ID NO:1, and a light chain variable region comprising SEQ ID NO:2.

In one embodiment, the disease or disorder is an inflammatory bowel disease (IBD). In some embodiments, the IBD is ulcerative colitis, Crohn's disease, ileitis, Celiac disease, nontropical Sprue, enteropathy associated with seronegative arthropathies, microscopic or collagenous colitis, eosinophilic gastroenteritis, or pouchitis resulting after proctocolectomy, and ileoanal anastomosis. In some embodiments, the inflammatory bowel disease is Crohn's disease or ulcerative colitis. Additional diseases which can be treated include, for example, primary sclerosing cholangitis (PSC), and graft-versus-host disease (GVHD).

Additionally, the invention also comprises the following embodiments:

1. A method of producing a composition comprising vedolizumab having a reduced level of basic vedolizumab isoform species, comprising:

providing a composition comprising vedolizumab at a pH greater than pH 6.5; and

incubating the composition comprising vedolizumab for a period of greater than 10 hours;

thereby producing a composition comprising vedolizumab having a reduced level of basic vedolizumab isoform species.

2. The method of item 1, wherein the incubation is performed at ambient temperature.
3. The method of item 1, wherein the incubation is performed at 15-30° C.
4. The method of item 1, wherein the incubation is performed at 20-25° C.
5. The method of any one of the previous items, wherein the composition comprising vedolizumab is provided at a pH of about 6.5-8.5.
6. The method of any one of the previous items, wherein the composition comprising vedolizumab is provided at a pH of about 7.0-8.0.
7. The method of any one of the previous items, wherein the composition comprising vedolizumab is provided at a pH of about 7.0-7.5.
8. The method of any one of the previous items, wherein the composition comprising vedolizumab is provided at a pH of about pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, or pH 8.5.
9. The method of any one of the previous items, wherein the composition comprising vedolizumab is incubated for a period of about 10-120 hours.
10. The method of any one of the previous items, wherein the composition comprising vedolizumab is incubated for a period of about 12-120 hours.
11. The method of any one of the previous items, wherein the composition comprising vedolizumab is incubated for a period of about 12-96 hours.
12. The method of any one of the previous items, wherein the composition comprising vedolizumab is incubated for a period of about 12-72 hours.
13. The method of any one of the previous items, wherein the composition comprising vedolizumab is incubated for a period of about 12-48 hours.
14. The method of any one of the previous items, wherein the composition comprising vedolizumab is incubated for a period of at least 12 hours.
15. The method of any one of the previous items, wherein the composition comprising vedolizumab is incubated for a period of about 24-120 hours.
16. The method of any one of the previous items, wherein the composition comprising vedolizumab is incubated for a period of about 24-96 hours.
17. The method of any one of the previous items, wherein the composition comprising vedolizumab is incubated for a period of about 24-72 hours.
18. The method of any one of the previous items, wherein the composition comprising vedolizumab is incubated for a period of about 24-48 hours.
19. The method of any one of the previous items, wherein the composition comprising vedolizumab is incubated for a period of about 12 hours, about 24 hours, about 36 hours, about 48 hours, about 72 hours, about 96 hours, or about 120 hours.
20. The method of any one of the previous items, wherein the composition comprising vedolizumab is derived from a mammalian cell culture expressing vedolizumab.
21. The method of item 20, wherein the mammalian cell culture is a Chinese Hamster Ovary (CHO) cell culture.
22. The method of item 21, wherein the CHO cell culture comprises CHO cells that lack dihydrofolate reductase (DHFR) expression.
23. The method of item 21, wherein the CHO cell culture comprises CHO cells that lack glutamine synthetase (GS) expression.
24. The method of any one of the previous items, wherein the method further comprises purifying the composition comprising vedolizumab from mammalian host cell protein (HCP) using one or more chromatographic separation steps selected from the group consisting of affinity chromatography, cation exchange chromatography, anion exchange chromatography, and ceramic hydroxyapatite (CHT) chromatography.
25. The method of item 24, wherein the composition comprising vedolizumab is purified using an affinity chromatography resin comprising Protein A.
26. The method of item 25, wherein the composition comprising vedolizumab is purified using affinity chromatography prior to the incubation.
27. The method of item 25, wherein the composition comprising vedolizumab is purified using affinity chromatography following the incubation.
28. The method of item 24, wherein the composition comprising vedolizumab is purified using cation exchange chromatography.
29. The method of item 28, wherein the composition comprising vedolizumab is purified using cation exchange chromatography prior to the incubation.
30. The method of item 28, wherein the composition comprising vedolizumab is purified using cation exchange chromatography following the incubation.
31. The method of item 24, wherein the composition comprising vedolizumab is purified using anion exchange chromatography.
32. The method of item 31, wherein the composition comprising vedolizumab is purified using anion exchange chromatography prior to the incubation.
33. The method of item 31, wherein the composition comprising vedolizumab is purified using anion exchange chromatography following the incubation.
34. The method of item 24, wherein the composition comprising vedolizumab is purified using CHT chromatography.
35. The method of item 34, wherein the composition comprising vedolizumab is purified using CHT chromatography prior to the incubation.
36. The method of item 34, wherein the composition comprising vedolizumab is purified using CHT chromatography following the incubation.
37. A composition comprising vedolizumab, wherein the composition is produced by the method of any one of items 1-36.
38. The composition of item 37, wherein the basic vedolizumab isoform species comprises less than 10% of the vedolizumab species present in the composition.
39. A method of producing a composition comprising vedolizumab having a reduced amount of host cell protein (HCP), comprising:

(a) contacting a sample containing vedolizumab and HCP with an anion exchange resin in the presence of a loading buffer, wherein the loading buffer has a conductivity of 11 mS/cm or less, such that HCP binds to the anion exchange resin; and

(b) collecting the flow through material from the anion exchange resin,

wherein the flow through material comprises vedolizumab and a reduced amount of HCP.

40. The method of item 39, wherein the loading buffer has a conductivity of 9 mS/cm to 11 mS/cm.
41. The method of item 39, wherein the loading buffer has a conductivity of 10 mS/cm or less.
42. The method of item 39, wherein the loading buffer has a conductivity of 9 mS/cm or less.
43. The method of item 39, wherein the loading buffer has a conductivity of about 9 mS/cm, 9.5 mS/cm, 10 mS/cm, 10.5 mS/cm, or 11 mS/cm.
44. The method of any one of items 39-42, wherein the HCP is a Chinese Hamster Ovary (CHO) cell protein.
45. The method of item 44, wherein the HCP is derived from a CHO cell that lacks dihydrofolate reductase (DHFR) expression.
46. The method of item 44, wherein the HCP is derived from a CHO cell that lacks glutamine synthetase (GS) expression.
47. The method of any one of items 39-46, further comprising contacting the anion exchange resin with a wash buffer.
48. The method of item 47, wherein the wash buffer has a conductivity of less than 11 mS/cm.
49. The method of item 47, wherein the wash buffer has a conductivity of 9 mS/cm to 11 mS/cm.
50. The method of item 47, wherein the wash buffer has the same conductivity as the loading buffer.
51. The method of any one of items 39-50, wherein the loading buffer comprises sodium chloride and/or sodium phosphate.
52. The method of any one of items 39-50, wherein the wash buffer comprises sodium chloride and/or sodium phosphate.
53. The method of any one of items 39-52, wherein the anion exchange resin is formatted as an anion exchange column or an anion exchange membrane.
54. The method of any one of items 39-53, wherein the anion exchange resin comprises a quaternary amine functional group.
55. The method of any one of items 39-54, wherein the sample containing vedolizumab and HCP is derived from a mammalian cell culture following one or more chromatographic separation steps.
56. The method of item 55, wherein the one or more chromatographic separation steps comprise one or more steps selected from the group consisting of affinity chromatography, cation exchange chromatography, and ceramic hydroxyapatite (CHT) chromatography.
57. The method of any one of items 39-56, wherein the amount of HCP in the eluate is 8 ppm or less, 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, or 2 ppm or less.
58. The method of any one of items 39-57, wherein the amount of HCP in the flow through material is reduced by at least 50% relative to the amount of HCP in flow through material produced when the method is performed using the same sample with a loading buffer having a conductivity greater than 12 mS/cm.
59. The method of any one of items 39-58, wherein the method further comprises processing the flow through material to exchange the elution buffer by a process comprising ultrafiltration and/or diafiltration to a buffer comprising one or more pharmaceutically acceptable carriers or excipients.
60. A composition comprising vedolizumab produced by the method of any one of items 39-59.
61. The composition of item 60, wherein the amount of HCP in the composition is 8 ppm or less, 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, or 2 ppm or less.
62. A method of increasing the yield of vedolizumab recovered following elution from a mixed mode chromatography resin, comprising equilibrating the mixed mode chromatography resin with an equilibration buffer, loading a solution comprising vedolizumab and a loading buffer onto the mixed mode chromatography resin such that vedolizumab binds the mixed mode chromatography resin, washing the mixed mode chromatography resin with a wash buffer, and eluting vedolizumab from the mixed mode chromatography resin with an elution buffer, wherein the equilibration buffer, the loading buffer, and/or the wash buffer have a pH at or below 7.0.
63. The method of item 62, wherein the equilibration buffer, the loading buffer, and/or the wash buffer have a pH of 6.0-7.0.
64. The method of item 62, wherein the equilibration buffer, the loading buffer, and/or the wash buffer have a pH of 6.5-7.0.
65. The method of item 62, wherein the equilibration buffer, the loading buffer, and/or the wash buffer have a pH of 6.6-6.8.
66. The method of any one of items 62-65, wherein the equilibration buffer, the loading buffer, and/or the wash buffer have a salt concentration of 30 mM to 70 mM.
67. The method of item 66, wherein the equilibration buffer, the loading buffer, and/or the wash buffer have a salt concentration of 40 mM to 70 mM.
68. The method of item 66, wherein the equilibration buffer, the loading buffer, and/or the wash buffer have a salt concentration of 50 mM to 65 mM.
69. The method of item 66, wherein the equilibration buffer, the loading buffer, and/or the wash buffer have a salt concentration of 55 mM-65 mM.
70. The method of any one of items 66-69, wherein the salt comprises sodium chloride and/or sodium phosphate.
71. The method of any one of items 62-65, wherein the equilibration buffer, the loading buffer, and/or the wash buffer have a sodium chloride concentration of 30 mM to 70 mM.
72. The method of item 71, wherein the equilibration buffer, the loading buffer, and/or the wash buffer have a sodium chloride concentration of 40 mM to 70 mM.
73. The method of item 71, wherein the equilibration buffer, the loading buffer, and/or the wash buffer have a sodium chloride concentration of 40 mM to 60 mM.
74. The method of item 71, wherein the equilibration buffer, the loading buffer, and/or the wash buffer have a sodium chloride concentration of 45 mM-55 mM.
75. The method of item 71, wherein the equilibration buffer, the loading buffer, and/or the wash buffer have a sodium chloride concentration of about 50 mM.
76. The method of any one of items 71-75, wherein the equilibration buffer, the loading buffer, and/or the wash buffer further comprise sodium phosphate.
77. The method of any one of items 62-76, wherein the equilibration buffer, the loading buffer, and/or the wash buffer have the same pH.
78. The method of any one of items 62-77, wherein the equilibration buffer, the loading buffer, and/or the wash buffer have the same salt concentration.
79. The method of any one of items 62-76, wherein the equilibration buffer, the loading buffer, and/or the wash buffer are the same buffer.
80. The method of any one of items 62-79, wherein the mixed mode resin is a ceramic hydroxyapatite resin.

### BRIEF DESCRIPTION OF THE DRAWINGS

FIG. 1 depicts the cation exchange (CEX)-high performance liquid chromatography (HPLC) profile of vedolizumab, with peaks representing acidic species, basic species, and the major isoform of vedolizumab indicated.

FIG. 2 depicts the elution profile of vedolizumab purified using a standard ceramic hydroxyapatite (CHT) equilibration and wash buffer after loading the CHT column with 27 mg/ml protein or 35 mg/ml protein.

FIG. 3 depicts the elution profile of vedolizumab purified using a standard CHT equilibration and wash buffer, or a reduced pH buffer, after loading the CHT column with 38 mg/ml protein.

### DETAILED DESCRIPTION OF THE INVENTION

Provided herein are methods for purifying an anti-α4β7 integrin antibody, such as vedolizumab, from a liquid solution, e.g., from a mammalian cell culture clarified harvest. The invention relates, inter alia, to purification methods for controlling the amount of product-related substances (e.g., basic and/or acidic isoform species) and/or process-related impurities (e.g., host cell proteins (HCPs), host cell nucleic acids, viruses, chromatographic materials, and/or media components) present in purified preparations of an anti-α4β7 integrin antibody, or antigen-binding fragment thereof, e.g., vedolizumab. Vedolizumab is a relatively hydrophobic antibody, which presents challenges in purification, particularly when the antibody is produced in large quantities at a level of purity necessary for therapeutic use.

### I. Definitions

In order that the present invention may be more readily understood, certain terms are first defined.

The cell surface molecule, “α4β7 integrin,” or “α4β7” (used interchangeably throughout) is a heterodimer of an α4 chain (CD49D, ITGA4) and a β7 chain (ITGB7). Human α4-integrin and β7-integrin genes GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)). An antibody that binds to α4β7 integrin is referred to herein as an “anti-α4β7 antibody”.

As used herein, an antibody, or antigen-binding fragment thereof, that has “binding specificity for the α4β7 complex” binds to α4β7, but not to α4β1 or α_{E}B7. Vedolizumab is an example of an antibody that has binding specificity for the α4β7 complex.

The term “about” denotes that the thereafter following value is no exact value but is the center point of a range that is +/−5% of the value of the value. If the value is a relative value given in percentages the term “about” also denotes that the thereafter following value is no exact value but is the center point of a range that is +/−5% of the value, whereby the upper limit of the range cannot exceed a value of 100%.

As used herein, the terms “aggregate” or “aggregates” refer to the association of two or more antibodies or antibody fragments. For example, an aggregate can be a dimer, trimer, tetramer, or a multimer greater than a tetramer, of antibodies and/or antibody fragments. Antibody aggregates can be soluble or insoluble. The association between the aggregated molecules may be either covalent or non-covalent without respect to the mechanism by which they are associated. The association may be direct between the aggregated molecules or indirect through other molecules that link them together. Examples of the latter include, but are not limited to disulfide linkages with other proteins, hydrophobic associations with lipids, charge associations with DNA, affinity associations with leached protein A, or mixed associations with multiple components. Aggregates can be irreversibly formed either during protein expression in cell culture, during protein purification in downstream processing, or during storage. The presence of aggregates in a solution can be determined using, for example, size exclusion chromatography (SEC) (e.g., SEC with UV detection, SEC with light scattering detection (SEC-LSD)), field flow fractionation, analytical ultracentrifugation sedimentation velocity, or capillary electrophoresis-sodium dodecyl sulfate (CE-SDS, reduced and non-reduced).

The term “antibody” as used herein, is intended to refer to an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the antibody has a fragment crystallizable (Fc) region. In certain embodiments, the antibody is an IgG1 isotype and has a kappa light chain.

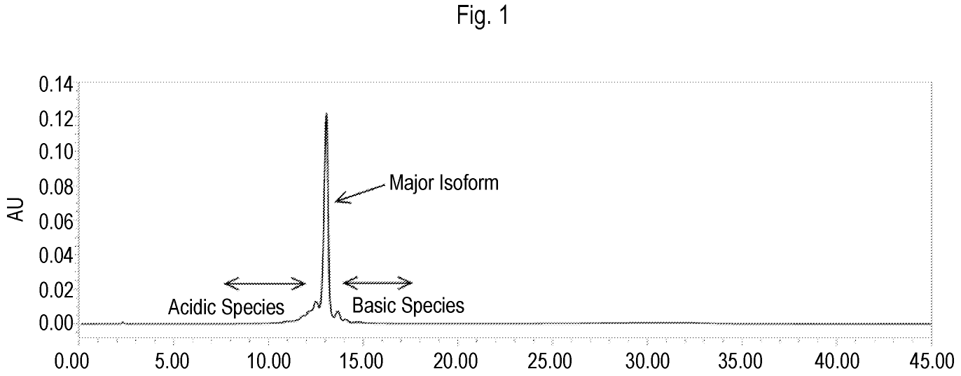

The terms “basic species” or “basic isoform species”, as used herein, refer to variants of an antibody, or antigen binding portion thereof, e.g., vedolizumab, which are characterized by an overall basic charge. Basic species of an antibody or antigen binding portion thereof can be detected by various methods known in the art, such as cation exchange-high performance liquid chromatography (CEX-HPLC), CEX-mass spectrometry, or isoelectric focusing. Basic species of an antibody may include, but are not limited to, charge variants, structural variants, and/or fragmentation variants. In some embodiments, a composition comprising an antibody, or antigen binding portion thereof, can comprise more than one type of basic isoform species. In some embodiments, multiple basic isoform species can be identified based on differences in retention time during CEX-HPLC separation. For example, when a composition comprising an antibody, e.g., vedolizumab, is analyzed using CEX-HPLC, one or more basic isoform peaks may be identified, each representing one or more basic isoform species of the antibody, as illustrated in FIG. 1. For example, in some embodiments, the basic isoform species is an isoform of the antibody in which an aspartic acid residue has undergone isomerization to succinimide. Host cell impurities, or other impurities that are not related to the antibody, or antigen binding portion thereof, by primary sequence, are not considered “basic species” or “basic isoform species” of the antibody, or antigen binding portion thereof.

The term “buffer,” as used herein, refers to an aqueous solution that resists changes in pH by the action of its acid-base conjugate components. A buffer is used to establish a specified set of conditions, e.g., biochemical conditions, to mediate control of a processing step or chromatographic support, such as a chromatography resin or membrane.

A “CDR” or “complementarity determining region” is a region of hypervariability interspersed within regions that are more conserved, termed “framework regions” (FR). As used herein, the term “antigen binding fragment” or “antigen binding portion” of an antibody refers to Fab, Fab′, F(ab′)₂, and Fv fragments, single chain antibodies, functional heavy chain antibodies (nanobodies), as well as any portion of an antibody having specificity toward at least one desired epitope, that competes with the intact antibody for specific binding (e.g., an isolated portion of a complementarity determining region having sufficient framework sequences so as to bind specifically to an epitope). Antigen binding fragments can be produced by recombinant techniques, or by enzymatic or chemical cleavage of an antibody.

A “chromatographic support”, as used herein, refers to a solid or porous matrix of a specific chemical composition or specific three-dimensional structure or on which specific chemical groups or macromolecules may be immobilized in order to perform chromatography, including affinity chromatography, gel filtration (size exclusion chromatography), or ion exchange chromatography. Examples of a chromatographic support include, but are not limited to, resin (e.g., agarose) or a membrane. A “chromatographic housing,” as used herein refers to a structure containing the chromatographic support. Examples of a chromatographic housing include a column or a cartridge, or other container.

The term “clarified harvest,” as used herein, refers to a liquid material containing a protein of interest, for example, an anti-α4β7 antibody, that has been extracted from cell culture, for example, a fermentation bioreactor, after undergoing one or more process steps to remove solid particles, such as cell debris and particulate impurities from the material. Following cell culture, the harvest is typically purified to remove cells and cellular debris using separation techniques, such as centrifugation and filtration. Initial clarification, particulate removal steps result in a “clarified harvest” that can be used, for example, in subsequent chromatographic steps (downstream processing). The clarified harvest is generally the starting material for downstream processing, such as downstream processing steps described herein.

As used herein, the terms “culture” and “cell culture” generally refer to the (upstream) process by which cells are grown under controlled conditions, generally outside of their natural environment. “Culturing” a cell refers to contacting a cell with a cell culture medium under conditions suitable to the survival and/or growth and/or proliferation of the cell. Cell culture, in certain embodiments, refers to methods for generating and maintaining a population of host cells capable of producing a recombinant protein of interest, e.g., an anti-α4β7 antibody, as well as the methods and techniques for the production and collection of the protein of interest. For example, once an expression vector has been incorporated into an appropriate host, e.g., a host cell in culture, the host can be maintained under conditions suitable for expression of the relevant nucleotide coding sequences, and the collection and purification of the desired recombinant protein. “Cell culture” can also refer to a solution containing cells.

As used herein, the term “downstream process” refers to one or more techniques used after the upstream process to purify the protein, e.g., antibody, of interest. For example, a downstream process technique includes purification of the protein product, using, for example, affinity chromatography, including Protein A affinity chromatography, ion exchange chromatography, such as anion or cation exchange chromatography, size exclusion chromatography, mixed mode chromatography, hydrophobic interaction chromatography (HIC), or displacement chromatography.

The term “elution solution” or “eluent,” as used herein, refers to an aqueous liquid formulated to displace a protein of interest, e.g., antibody from a chromatographic support, e.g., resin or membrane. In one embodiment, an elution solution has biochemical characteristics different from the equilibration and/or wash solution, such that the protein, e.g., antibody, of interest prefers to associate with the elution solution, rather than with the chromatographic support, e.g., resin or membrane.

The term “equilibration solution,” as used herein, refers to an aqueous liquid formulated to create the initial operating conditions for a processing step or chromatographic support, such as a chromatographic operation. An equilibration solution is used to prepare, for example, a solid phase, e.g., a chromatographic support, e.g., resin or membrane, for loading the protein, e.g., antibody, of interest.

A “flow-through operation,” as used herein, e.g., in relation to a chromatographic step, refers to a process by which the protein elutes during loading and a wash, while impurities bind and remain associated with the chromatographic support.

The term “high molecular weight” or “HMW” is used to indicate an antibody complex having a molecular weight greater than a monomer antibody. In one embodiment, a HMW aggregate has a molecular weight greater than about 147 kDa. The presence of high molecular weight aggregates may be determined by standard methods known in the art, e.g., size-exclusion chromatography (SEC).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable CDR loops correspond to those of a non-human antibody and all or substantially all of the FRs are those of a human antibody sequence. The humanized antibody optionally also will comprise at least a portion of an antibody constant region (Fc), typically that of a human antibody. For further details, see Jones et al., Nature 321:522-525 (1986); Riechmann et al., Nature 332:323-329 (1988); and Presta, Curr. Op. Struct. Biol. 2:593-596 (1992).

The term “impurity,” as used herein with respect to impurities contained in a solution comprising an antibody to be purified, includes both process-related impurities and product-related impurities. The term “process-related impurity,” as used herein, refers to an impurity (or impurities) that are present in a composition, e.g., a solution, comprising a protein but are not derived from the protein itself. For example, process-related impurities include, but are not limited to, cell culture media components, host cell components (such as proteins (HCPs), host cell nucleic acids, or lipid-containing subcellular structures or fragments thereof), viruses, trace metals or ions from the buffers, leachable materials from the material-handling vessels or chromatographic support. Process-related impurities can be formed during the preparation (upstream and/or downstream processing) of the protein, e.g., the antibody. As used herein, the term “host cell impurity” refers to any proteinaceous, nucleic acid contaminant, lipid contaminant, or by-product introduced by the host cell line, cell cultured fluid, or cell culture. The term “host cell protein” refers to a proteinaceous by-product introduced by the host cell line, cell culture fluid, or cell culture. Examples of impurities include, but are not limited to, Chinese Hamster Ovary Protein (CHOP), E. coli protein, yeast protein, simian COS protein, or myeloma cell protein (e.g., NS0 protein (mouse plastocytoma cells derived from a BALB/c mouse)). A host cell protein does not include a protein of interest that is produced in a host cell expression system. For example, when a CHO cell is used to produce a recombinant antibody, or fragment thereof, the term “host cell protein” encompasses proteins derived from the CHO cell, other than the recombinant antibody or fragment thereof. The term “product-related impurities,” as used herein, includes impurities derived from the protein, e.g., antibody, of interest itself. For example, product-related impurities include, but are not limited to, aggregates, mis-folded species, oxidized or deamidated species or low molecular weight fragments, of the antibody of interest.

As used herein, the term “recombinant antibody” refers to an antibody produced as the result of the transcription and translation of a gene(s) carried on a recombinant expression vector(s) that has been introduced into a host cell, e.g. a mammalian host cell. In certain embodiments the recombinant protein is an antibody of an isotype selected from group consisting of: IgG (e.g., IgG1, IgG2, IgG3, IgG4), IgM, IgA1, IgA2, IgD, or IgE. In certain embodiments the recombinant antibody is an IgG1.

The term “recombinant host cell” (used interchangeably herein with the term “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Further, it should be understood that unless specified otherwise, where the term “cell” is used, e.g., host cell or mammalian cell or mammalian host cell, it is intended to include a population of cells.

“Substantially purified” with regard to the desired protein means that the purified sample comprising the protein comprises at least 90%, at least 91%, at least 92%, at least 93%, at least 94%, at least 95%, at least 96%, at least 97%, at least 97.5%, at least 98%, at least 98.5%, or at least 99% of the desired recombinant protein with less than 3%, less than 2.5%, less than 2%, less than 1.5%, less than 1%, or less than 0.5% of impurities.

As used herein, the term “upstream process” in the context of protein, e.g., antibody, preparation, refers to activities involving the production and collection of proteins (e.g. antibodies) from host cells (e.g., upon cell culture to produce a protein of interest, e.g., antibody).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “wash” or “wash solution,” as used herein, refers to an aqueous liquid formulated to displace unbound contaminants from a chromatographic support, such as resin or membrane. In some embodiments, a wash is passed over a solid support, e.g., a resin or membrane, following loading with a protein, e.g., antibody, of interest and prior to elution of the protein, e.g., antibody, of interest. In one embodiment, a wash has biochemical characteristics similar to the equilibration solution.

### II. Anti-α4β7 Integrin Antibodies

The methods disclosed herein may be used to produce large quantities of a highly purified composition comprising an anti-α4β7 integrin antibody. As will be apparent, any of the methods for producing an anti-α4β7 integrin antibody described herein can be used individually, or in combination. In exemplary embodiments, the antibody is vedolizumab, or an antibody having the antigen binding region(s) of vedolizumab. Vedolizumab is also known by its trade name ENTYVIO® (Takeda Pharmaceuticals, Inc.). Vedolizumab is a humanized antibody that comprises human IgG1 framework and constant regions, and antigen-binding CDRs from the murine antibody Act-1. Vedolizumab CDRs, variable regions, and mutated Fc region (mutated to eliminate Fc effector functions) are described in U.S. Pat. No. 7,147,851, incorporated by reference herein in its entirety.

Vedolizumab is a humanized monoclonal antibody that specifically binds to α4β7 integrin, e.g., the α4β7 complex. Vedolizumab blocks the interaction of α4β7 integrin with mucosal addressin cell adhesion molecule-1 (MAdCAM-1), and inhibits the migration of memory T-lymphocytes across the endothelium into inflamed gastrointestinal parenchymal tissue. Vedolizumab does not bind to or inhibit function of the integrins α4β1 or αEβ7, and does not antagonize the interaction of α4 integrins with vascular cell adhesion molecule-1 (VCAM-1).

The α4β7 integrin is expressed on the surface of a discrete subset of memory T-lymphocytes that preferentially migrate into the gastrointestinal tract. MAdCAM-1 is mainly expressed on gut endothelial cells and plays a critical role in the homing of T-lymphocytes to gut lymph tissue. The interaction of the α4β7 integrin with MAdCAM-1 has been implicated as an important contributor to mucosal inflammation, such as the chronic inflammation that is a hallmark of ulcerative colitis and Crohn's disease. Vedolizumab may be used to treat inflammatory bowel disease, including Crohn's disease and ulcerative colitis, pouchitis, including chronic pouchitis, graft-versus host disease, and HIV.

The heavy chain variable region of vedolizumab is provided herein as SEQ ID NO:1, and the light chain variable region of vedolizumab is provided herein as SEQ ID NO:5. Vedolizumab comprises a heavy chain variable region comprising a CDR1 of SEQ ID NO:2, a CDR2 of SEQ ID NO:3, and a CDR3 of SEQ ID NO:4. Vedolizumab comprises a light chain variable region comprising a CDR1 of SEQ ID NO:6, a CDR2 of SEQ ID NO:7 and CDR3 of SEQ ID NO:8. In one embodiment, the antibody comprises a heavy chain comprising the amino acid sequence of SEQ ID NO: 9, and a light chain comprising the amino acid sequence of SEQ ID NO: 10. Vedolizumab and the sequences of vedolizumab are also described in U.S. Patent Publication No. 2014/0341885 and U.S. Patent Publication No. 2014-0377251, the entire contents of each which are expressly incorporated herein by reference in their entireties. The methods disclosed herein can be performed using an antibody comprising binding regions, e.g., CDRs or variable regions, set forth above.

The methods of the invention are useful for producing an anti-α4β7 antibody, particularly vedolizumab or an antibody having the binding regions, i.e., CDRs or variable regions, of vedolizumab, in mammalian cells.

Exemplary Strategies for Antibody Production

In certain embodiments, the methods described herein can be performed in conjunction with one or more additional steps to facilitate the production and/or purification of vedolizumab, including one or more steps described below. For long-term, high-yield production of recombinant proteins such as vedolizumab, mammalian host cells can be engineered to stably express an anti-α4β7 antibody (e.g., vedolizumab). An exemplary cell culture process, and considerations for production of monoclonal antibodies such as vedolizumab, is described in Li et al. (2010) mAbs 2:5, 466-477, and Birch and Racher (2006) Adv. Drug Delivery Rev. 58:671-685, the entire contents of which are incorporated by reference herein.

In certain embodiments, primary recovery can proceed by sequentially employing pH reduction, centrifugation, and filtration steps to remove cells and cell debris (including HCPs) from the production bioreactor harvest. In certain embodiments, the present invention is directed to subjecting a sample mixture from said primary recovery to one or more of affinity chromatography, anion exchange (AEX), cation exchange (CEX), hydrophobic interaction chromatography (HIC), ceramic hydroxyapatite chromatography (CHT), and/or mixed mode (MM) purification steps. In some embodiments, the order of steps can impact the resulting quality of the antibody composition, by modulating the levels of aggregates, impurities, or isoforms.

In one exemplary embodiment, a composition comprising vedolizumab can be purified using a process comprising steps in the order: affinity chromatography (e.g., Protein A), mixed mode, cation exchange, anion exchange.

In another exemplary embodiment, a composition comprising vedolizumab can be purified using a process comprising steps in the order: affinity chromatography (e.g., Protein A), mixed mode, anion exchange, cation exchange.

In another exemplary embodiment, a composition comprising vedolizumab can be purified using a process comprising steps in the order: affinity chromatography (e.g., Protein A), cation exchange, mixed mode, anion exchange.

In another exemplary embodiment, a composition comprising vedolizumab can be purified using a process comprising steps in the order: affinity chromatography (e.g., Protein A), anion exchange, mixed mode, cation exchange.

In another exemplary embodiment, a composition comprising vedolizumab can be purified using a process comprising steps in the order: mixed mode, affinity chromatography (e.g., Protein A), anion exchange, cation exchange.

In another exemplary embodiment, a composition comprising vedolizumab can be purified using a process comprising steps in the order: affinity chromatography (e.g., Protein A), CHT chromatography, cation exchange, anion exchange.

In another exemplary embodiment, a composition comprising vedolizumab can be purified using a process comprising steps in the order: affinity chromatography (e.g., Protein A), CHT chromatography, anion exchange, cation exchange.

In another exemplary embodiment, a composition comprising vedolizumab can be purified using a process comprising steps in the order: affinity chromatography (e.g., Protein A), cation exchange, CHT chromatography, anion exchange.

In another exemplary embodiment, a composition comprising vedolizumab can be purified using a process comprising steps in the order: affinity chromatography (e.g., Protein A), hydrophobic interaction chromatography, cation exchange, anion exchange.

In another exemplary embodiment, a composition comprising vedolizumab can be purified using a process comprising steps in the order: affinity chromatography (e.g., Protein A), hydrophobic interaction chromatography, anion exchange, cation exchange.

In another exemplary embodiment, a composition comprising vedolizumab can be purified using a process comprising steps in the order: affinity chromatography (e.g., Protein A), anion exchange, hydrophobic interaction chromatography, cation exchange.

In another exemplary embodiment, a composition comprising vedolizumab can be purified using a process comprising steps in the order: affinity chromatography (e.g., Protein A), cation exchange, hydrophobic interaction chromatography, anion exchange.

In another exemplary embodiment, a composition comprising vedolizumab can be purified using a process comprising steps in the order: affinity chromatography (e.g., Protein A), cation exchange, anion exchange, hydrophobic interaction chromatography.

In another exemplary embodiment, a composition comprising vedolizumab can be purified using a process comprising steps in the order: affinity chromatography (e.g., Protein A), anion exchange, cation exchange, hydrophobic interaction chromatography.

In another exemplary embodiment, a composition comprising vedolizumab can be purified using a process comprising steps in the order: hydrophobic interaction chromatography, affinity chromatography (e.g., Protein A), anion exchange, cation exchange.

It is to be understood that the purification steps are not necessarily immediately adjacent to each other; various other process steps such as filtration or viral reduction steps may be inserted between the chromatography steps without disturbing the effect of chromatography step order on charge profile.

In order to modulate the level of basic isoform species present in a vedolizumab composition, an incubation step can be incorporated between any of the foregoing purification steps, as described herein. Additionally or alternatively, the purification process can be adapted to minimize the duration of time that the antibody is exposed to low pH conditions, as described herein.

In order to modulate the level of host cell protein present in a vedolizumab composition, AEX can be performed using low conductivity buffers as described herein, at any stage in the vedolizumab purification process employing anion exchange chromatography.

In order to modulate the yield of vedolizumab in a purification process comprising CHT, the CHT conditions described herein can be used at any stage in the vedolizumab purification process employing ceramic hydroxyapatite chromatography.

Certain embodiments of the present invention will include further purification steps. Examples of additional purification procedures which can be performed prior to, during, or following the ion exchange chromatography method include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxylapatite chromatography, gel electrophoresis, dialysis, and affinity chromatography (e.g., using protein G, an antibody, a specific substrate, ligand or antigen as the capture reagent).

In certain embodiments the unbound flow through and wash fractions can be further fractionated, and a combination of fractions providing a target product purity can be pooled.

In certain embodiments the protein concentration can be adjusted to achieve a differential partitioning behavior between the antibody product and the product-related substances such that the purity and/or yield can be further improved. In certain embodiments the loading can be performed at different protein concentrations during the loading operation to improve the product quality/yield of any particular purification step.

In certain embodiments the column temperature can be independently varied to improve the separation efficiency and/or yield of any particular purification step.

In certain embodiments, the loading and washing solution matrices can be different or composed of mixtures of chemicals, while achieving similar “resin interaction” behavior such that the above novel separation can be effected. For example, but not by way of limitation, the loading and washing solutions can be different, in terms of ionic strength or pH, while remaining substantially similar in function in terms of the washout of the product achieved during the wash step. In certain embodiments, additives such as amino acids, sugars, PEG, etc. can be added to the load or wash steps to modulate the partitioning behavior to achieve the separation efficiency and/or yield.

In certain embodiments, the loading & washing steps can be controlled by in-line, at-line or off-line measurement of the product related impurity/substance levels, either in the column effluent, or the collected pool or both, so as to achieve the target product quality and/or yield. In certain embodiments, the loading concentration can be dynamically controlled by in-line or batch or continuous dilutions with solutions or other solutions to achieve the partitioning necessary to improve the separation efficiency and/or yield.

Certain embodiments of the present invention employ ultrafiltration and diafiltration steps to further concentrate and formulate the protein of interest, e.g., an antibody product. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). One filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 μm. By employing filters having such small pore size, the volume of the sample can be reduced through permeation of the sample solution through the filter membrane pores while proteins, such as antibodies, are retained above the membrane surface.

Diafiltration is a method of using membrane filters to remove and exchange salts, sugars, and non-aqueous solvents, to separate free from bound species, to remove low molecular-weight species, and/or to cause the rapid change of ionic and/or pH environments. Microsolutes are removed most efficiently by adding solvent to the solution being diafiltered at a rate approximately equal to the permeate flow rate. This washes away microspecies from the solution at a constant volume, effectively purifying the retained protein of interest. In certain embodiments of the present invention, a diafiltration step is employed to exchange the various solutions used in connection with the instant invention, optionally prior to further chromatography or other purification steps, as well as to remove impurities from the protein preparations.

The ultrafiltration/diafiltration (UF/DF) of vedolizumab can be performed in a choice of apparatus and membrane, for example polyethersulfone or regenerated cellulose, e.g., ULTRACEL® or BIOMAX® membrane, in a PELLICON® cassette (MilliporeSigma, Burlington Mass.). In one embodiment, UF is performed using a cellulose membrane cast on a microporous polyethylene membrane having a molecular weight cut off of 30 kDa.

### III. Preparation of Vedolizumab Compositions Containing Altered Levels of Basic Isoform Species

Preparations of vedolizumab from mammalian host cells typically contain small quantities of acidic and/or basic isoform species, in addition to the major (or main) isoform of vedolizumab. Acidic and basic isoform species can be quantified by methods known in the art, including, for example, cation exchange-high pressure liquid chromatography (CEX-HPLC). Acidic and basic vedolizumab isoforms can be resolved from the major isoform based on differences in retention time on a CEX resin. In general, as depicted in FIG. 1, acidic vedolizumab isoform species present in a liquid preparation of vedolizumab have a shorter retention time relative to the major isoform, while basic vedolizumab isoform species have a longer retention time relative to the major isoform. A preparation of vedolizumab may comprise more than one acidic species, and/or more than one basic species, which have slight variations in charge and consequently elute from a CEX resin with different retention times. Multiple basic peaks are referenced herein in relation to their retention time on a CEX resin, whereby the first basic isoform peak to elute from the CEX resin following the major isoform peak is referred to herein as “basic peak 1”, the second basic isoform peak to elute from the CEX resin following the major isoform peak is referred to herein as “basic peak 2”, the third basic isoform peak to elute from the CEX resin following the major isoform peak is referred to herein as “basic peak 3”, and so forth.

In a pharmaceutical antibody preparation, it can be desirable to maximize the percentage of the antibody present as the major isoform, while minimizing the percentage of basic and/or acidic isoforms. The present invention provides, in one aspect, a method of modulating the percentage of basic vedolizumab isoforms in a composition comprising vedolizumab. This method is based on the surprising finding that the distribution of vedolizumab isoforms can be modulated by changes in pH. The basic isoform of vedolizumab is particularly sensitive to pH fluctuations. As described herein, this pH dependent modulation in isoform distribution is driven, at least in part, by fluctuations in the level of basic peak 2, and accompanying fluctuations in the level of the vedolizumab major isoform.

Without wishing to be bound by theory, and based at least in part on the findings provided herein, at least two basic isoform species are believed to be present in some vedolizumab preparations. The first, eluting from a CEX resin as “basic peak 1”, is attributable to the presence of a lysine residue at the carboxy terminus of the IgG heavy chain. The second, eluting from a CEX resin as “basic peak 2”, is attributable to the isomerization of one or more aspartic acid residues in the antibody to form a succinimide intermediate. In certain embodiments, one or more aspartic acid residues has undergone isomerization to form a succinimide intermediate. A glycine or serine residue adjacent to an aspartic acid at the “n+1 position” (adjacent and one amino acid closer to the carboxy terminus) can favor isomerization of the aspartic acid residue to succinimide. Identification of this variant of vedolizumab, which comprises succinimide in place of aspartic acid at residue 102 of SEQ ID NO:1, allows this basic isoform variant, in some embodiments, to be reduced, minimized or removed in a vedolizumab preparation. In some embodiments, this basic isoform variant can be removed by methods including, for example, fractionating a preparation comprising vedolizumab (e.g., on a CEX resin), and removing (or failing to collect) those fractions containing succinimide in place of aspartic acid at residue 102 of SEQ ID NO:1, also identifiable as the fraction of vedolizumab eluting from a CEX resin as “basic peak 2”. In some embodiments, the level of this basic isoform variant (referred to herein as the “succinimide variant” or alternatively as the “basic isoform peak 2” or “BP2” variant) can be minimized during vedolizumab production and purification, by controlling the pH exposure of the antibody. In some embodiments, a composition comprising reduced levels of this basic isoform variant can comprise a corresponding increase in the relative proportion of the vedolizumab major isoform. Accordingly, in some embodiments, a composition comprising reduced levels of this basic isoform variant can have increased potency relative to a composition comprising increased levels of this basic isoform variant.

In one embodiment, provided herein are methods of purifying vedolizumab with a reduced level of basic isoform species, wherein vedolizumab is maintained at or above pH 5.5, at or above pH 5.6, at or above pH 5.7, at or above pH 5.8, at or above pH 5.9, at or above pH 6.0, at or above pH 6.1, at or above pH 6.2, at or above pH 6.3, at or above pH 6.4, or at or above pH 6.5 for at least 60%, at least 65%, at least 70%, at least 75%, at least 80%, at least 85%, at least 90%, at least 95%, at least 97%, at least 98%, or at least 99% of the total time between primary recovery of vedolizumab from a cell culture harvest to formulation of vedolizumab in a pharmaceutically acceptable carrier by ultrafiltration/diafiltration (UF/DF). By maintaining vedolizumab at a pH at or above pH 5.5, pH 5.6, pH 5.7, pH 5.8, pH 5.9, pH 6.0, pH 6.1, pH 6.2, pH 6.3, pH 6.4 or pH 6.5 for the majority of the purification process, basic vedolizumab isoform species can be reduced, relative to an equivalent purification process in which vedolizumab is at a pH below pH 5.5-6.5 for a significant time, for example, greater than 5%, greater than 10%, greater than 15%, greater than 20%, greater than 25%, greater than 30%, greater than 35%, or greater than 40% of the total time between primary recovery of vedolizumab from a cell culture harvest to formulation of vedolizumab in a pharmaceutically acceptable carrier by ultrafiltration/diafiltration (UF/DF). In some embodiments, the method is performed at commercial manufacturing scale, e.g., using a preparation of vedolizumab derived from a cell culture harvest produced at 1000 L scale, 2000 L scale, 3000 L scale, 4000 L scale, or 5000 L scale (e.g., at least 3000 L scale).

Accordingly, in one aspect, the invention provides a method of producing a low basic species composition of an anti-α4β7 antibody, or an antigen binding portion thereof, comprising (i) providing a clarified cell culture harvest obtained from a culture of recombinant host cells expressing the anti-α4β7 antibody or an antigen binding portion thereof, (ii) and purifying the anti-α4β7 antibody or an antigen binding portion thereof from the cell culture harvest, wherein the antibody is exposed to a pH at or below 3.5 (e.g., pH 2.5-3.5, pH below 3.0, or pH below 3.5) for no more than 20 minutes, no more than 30 minutes, no more than 45 minutes, no more than 1 hour, no more than 3 hours, no more than 5 hours, no more than 7 hours, no more than 10 hours, or no more than 12 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 3.5 (e.g., pH 2.5-3.5, pH below 3.0, or pH below 3.5) for a longer duration of time, i.e., greater than 20 minutes, greater than 30 minutes, greater than 45 minutes, greater than 1 hour, greater than 3 hours, greater than 5 hours, greater than 7 hours, greater than 10 hours, or greater than 12 hours. In some embodiments, the low basic species composition comprises a lower level of BP2 relative to the control. In some embodiments, the antibody or antigen binding portion thereof comprises a heavy chain variable region comprising the amino acid sequence set forth in SEQ ID NO:1, and a light chain variable region comprising the amino acid sequence set forth in SEQ ID NO:5. In some embodiments, the antibody or antigen binding portion thereof is vedolizumab, or an antigen binding portion thereof. In some embodiments, the step of purifying the anti-α4β7 antibody or an antigen binding portion thereof comprises one or more of Protein A chromatography, anion exchange chromatography, cation exchange chromatography, mixed mode chromatography, hydrophobic interaction chromatography, and combinations thereof. In some embodiments, the host cells expressing the anti-α4β7 antibody or an antigen binding portion thereof are GS-CHO cells. In other embodiments, the host cells are DHFR-CHO cells. In some embodiments, the low basic species composition comprises less than 16%, less than 15%, less than 14%, less than 13%, less than 12%, less than 11%, less than 10%, or less than 9% basic isoform species. In some embodiments, the low basic species composition comprises less than 4%, less than 3%, less than 2%, or less than 1% basic isoform peak 2.

In another aspect, the invention provides a method of producing a low basic species composition of an anti-α4β7 antibody, or an antigen binding portion thereof, comprising (i) providing a clarified cell culture harvest obtained from a culture of recombinant host cells expressing the anti-α4β7 antibody or an antigen binding portion thereof, (ii) and purifying the anti-α4β7 antibody or an antigen binding portion thereof from the cell culture harvest, wherein the antibody is exposed to a pH at or below 4.0 (e.g., pH 3.6 to 4.0), for no more than 20 minutes, no more than 30 minutes, no more than 45 minutes, no more than 1 hour, no more than 3 hours, no more than 5 hours, no more than 10 hours, no more than 12 hours, no more than 15 hours, no more than 18 hours, or no more than 24 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 4.0 (e.g., pH 3.6-4.0) for a longer duration of time, i.e., greater than 20 minutes, greater than 30 minutes, greater than 45 minutes, greater than 1 hour, greater than 3 hours, greater than 5 hours, greater than 10 hours, greater than 12 hours, greater than 15 hours, greater than 18 hours, or greater than 24 hours. In some embodiments, the low basic species composition comprises a lower level of BP2 relative to the control. In some embodiments, the antibody or antigen binding portion thereof comprises a heavy chain variable region comprising the amino acid sequence set forth in SEQ ID NO:1, and a light chain variable region comprising the amino acid sequence set forth in SEQ ID NO:5. In some embodiments, the antibody or antigen binding portion thereof is vedolizumab, or an antigen binding portion thereof. In some embodiments, the step of purifying the anti-α4β7 antibody or an antigen binding portion thereof comprises one or more of Protein A chromatography, anion exchange chromatography, cation exchange chromatography, mixed mode chromatography, hydrophobic interaction chromatography, and combinations thereof. In some embodiments, the host cells expressing the anti-α4β7 antibody or an antigen binding portion thereof are GS-CHO cells. In other embodiments, the host cells are DHFR-CHO cells. In some embodiments, the low basic species composition comprises less than 16%, less than 15%, less than 14%, less than 13%, less than 12%, less than 11%, less than 10%, or less than 9% basic isoform species. In some embodiments, the low basic species composition comprises less than 4%, less than 3%, less than 2%, or less than 1% basic isoform peak 2.

In another aspect, the invention provides a method of producing a low basic species composition of an anti-α4β7 antibody, or an antigen binding portion thereof, comprising (i) providing a clarified cell culture harvest obtained from a culture of recombinant host cells expressing the anti-α4β7 antibody or an antigen binding portion thereof, (ii) and purifying the anti-α4β7 antibody or an antigen binding portion thereof from the cell culture harvest, wherein the antibody is exposed to a pH at or below 4.5 (e.g., pH 4.1-4.5) for no more than 3 hours, no more than 5 hours, no more than 10 hours, or no more than 12 hours, no more than 18 hours, no more than 24 hours, no more than 36 hours, no more than 48 hours, no more than 72 hours, or no more than 96 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 4.5 (e.g., pH 4.1-4.5) for a longer duration of time, i.e., greater than 3 hours, greater than 5 hours, greater than 10 hours, greater than 12 hours, greater than 18 hours, greater than 24 hours, greater than 36 hours, greater than 48 hours, greater than 72 hours, or greater than 96 hours. In some embodiments, the low basic species composition comprises a lower level of BP2 relative to the control. In some embodiments, the antibody or antigen binding portion thereof comprises a heavy chain variable region comprising the amino acid sequence set forth in SEQ ID NO:1, and a light chain variable region comprising the amino acid sequence set forth in SEQ ID NO:5. In some embodiments, the antibody or antigen binding portion thereof is vedolizumab, or an antigen binding portion thereof. In some embodiments, the step of purifying the anti-α4β7 antibody or an antigen binding portion thereof comprises one or more of Protein A chromatography, anion exchange chromatography, cation exchange chromatography, mixed mode chromatography, hydrophobic interaction chromatography, and combinations thereof. In some embodiments, the host cells expressing the anti-α4β7 antibody or an antigen binding portion thereof are GS-CHO cells. In other embodiments, the host cells are DHFR-CHO cells. In some embodiments, the low basic species composition comprises less than 16%, less than 15%, less than 14%, less than 13%, less than 12%, less than 11%, less than 10%, or less than 9% basic isoform species. In some embodiments, the low basic species composition comprises less than 4%, less than 3%, less than 2%, or less than 1% basic isoform peak 2.

In another aspect, the invention provides a method of producing a low basic species composition of an anti-α4β7 antibody, or an antigen binding portion thereof, comprising (i) providing a clarified cell culture harvest obtained from a culture of recombinant host cells expressing the anti-α4β7 antibody or an antigen binding portion thereof, (ii) and purifying the anti-α4β7 antibody or an antigen binding portion thereof from the cell culture harvest, wherein the antibody is exposed to a pH at or below 5.0 (e.g., pH 4.6-5.0) for no more than 3 hours, no more than 5 hours, no more than 10 hours, or no more than 12 hours, no more than 18 hours, no more than 24 hours, no more than 36 hours, no more than 48 hours, no more than 72 hours, or no more than 96 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 5.0 (e.g., pH 4.6-5.0) for a longer duration of time, i.e., greater than 3 hours, greater than 5 hours, greater than 10 hours, greater than 12 hours, greater than 18 hours, greater than 24 hours, greater than 36 hours, greater than 48 hours, greater than 72 hours, or greater than 96 hours. In some embodiments, the low basic species composition comprises a lower level of BP2 relative to the control. In some embodiments, the antibody or antigen binding portion thereof comprises a heavy chain variable region comprising the amino acid sequence set forth in SEQ ID NO:1, and a light chain variable region comprising the amino acid sequence set forth in SEQ ID NO:5. In some embodiments, the antibody or antigen binding portion thereof is vedolizumab, or an antigen binding portion thereof. In some embodiments, the step of purifying the anti-α4β7 antibody or an antigen binding portion thereof comprises one or more of Protein A chromatography, anion exchange chromatography, cation exchange chromatography, mixed mode chromatography, hydrophobic interaction chromatography, and combinations thereof. In some embodiments, the host cells expressing the anti-α4β7 antibody or an antigen binding portion thereof are GS-CHO cells. In other embodiments, the host cells are DHFR-CHO cells. In some embodiments, the low basic species composition comprises less than 16%, less than 15%, less than 14%, less than 13%, less than 12%, less than 11%, less than 10%, or less than 9% basic isoform species. In some embodiments, the low basic species composition comprises less than 4%, less than 3%, less than 2%, or less than 1% basic isoform peak 2.

In another aspect, the invention provides a method of producing a low basic species composition of an anti-α4β7 antibody, or an antigen binding portion thereof, comprising (i) providing a clarified cell culture harvest obtained from a culture of recombinant host cells expressing the anti-α4β7 antibody or an antigen binding portion thereof, (ii) and purifying the anti-α4β7 antibody or an antigen binding portion thereof from the cell culture harvest, wherein the antibody is exposed to a pH at or below 5.5 (e.g., pH 5.1-5.5) for no more than 3 hours, no more than 5 hours, no more than 10 hours, or no more than 12 hours, no more than 18 hours, no more than 24 hours, no more than 36 hours, no more than 48 hours, no more than 72 hours, or no more than 96 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 5.5 (e.g., pH 5.1-5.5) for a longer duration of time, i.e., greater than 3 hours, greater than 5 hours, greater than 10 hours, greater than 12 hours, greater than 18 hours, greater than 24 hours, greater than 36 hours, greater than 48 hours, greater than 72 hours, or greater than 96 hours. In some embodiments, the low basic species composition comprises a lower level of BP2 relative to the control. In some embodiments, the antibody or antigen binding portion thereof comprises a heavy chain variable region comprising the amino acid sequence set forth in SEQ ID NO:1, and a light chain variable region comprising the amino acid sequence set forth in SEQ ID NO:5. In some embodiments, the antibody or antigen binding portion thereof is vedolizumab, or an antigen binding portion thereof. In some embodiments, the step of purifying the anti-α4β7 antibody or an antigen binding portion thereof comprises one or more of Protein A chromatography, anion exchange chromatography, cation exchange chromatography, mixed mode chromatography, hydrophobic interaction chromatography, and combinations thereof. In some embodiments, the host cells expressing the anti-α4β7 antibody or an antigen binding portion thereof are GS-CHO cells. In other embodiments, the host cells are DHFR-CHO cells. In some embodiments, the low basic species composition comprises less than 16%, less than 15%, less than 14%, less than 13%, less than 12%, less than 11%, less than 10%, or less than 9% basic isoform species. In some embodiments, the low basic species composition comprises less than 4%, less than 3%, less than 2%, or less than 1% basic isoform peak 2.

In some embodiments, the methods provided herein are performed at commercial manufacturing scale, e.g., using a preparation of vedolizumab derived from a cell culture harvest produced at 1000 L scale, 2000 L scale, 3000 L scale, 4000 L scale, or 5000 L scale (e.g., at least 3000 L scale).

In some aspects, the methods described herein can be used to modulate the level of basic vedolizumab isoforms in a composition, e.g., a liquid composition, comprising vedolizumab. The methods can be used, in some embodiments, to produce a vedolizumab composition having low levels of basic vedolizumab species.

In one aspect, the invention provides a method of reducing the level of basic vedolizumab isoform species in a composition comprising vedolizumab, by incubating the composition at a pH of greater than pH 6.5 for a period of time sufficient for the level of basic vedolizumab isoform species to be reduced. In one embodiment, the method is performed at ambient temperature, e.g., 20-25° C. In other embodiments, the method is performed at 2-8° C.

In another aspect, the invention provides a method of producing a composition comprising vedolizumab having a reduced level of basic vedolizumab isoform species. The method can comprise providing a composition containing vedolizumab at a pH at or above pH 6.5, and incubating the composition comprising vedolizumab for a period of time sufficient for the level of basic vedolizumab isoform species to be reduced, thereby producing a composition comprising vedolizumab having a reduced level of basic vedolizumab isoform species.

In order to efficiently reduce the level of basic vedolizumab isoform species in a vedolizumab composition, the incubation is preferably performed at a pH at or above pH 6.5.

In exemplary embodiments, the pH of the vedolizumab composition is about pH 6.5-9.0. For example, the pH of the vedolizumab composition can be in the range of about pH 6.5-9.0, pH 6.5-8.5, pH 6.5-8.0, pH 6.5-7.5, or pH 6.5-7.0. Alternatively, the pH of the vedolizumab composition can be in the range of about pH 7.0-9.0, pH 7.5-9.0, pH 8.0-9.0, or pH 8.5-9.0. In other embodiments, the pH of the vedolizumab composition can be in the range of about pH 6.5-7.5, for example, pH 6.6-7.3, pH 6.6-7.5, pH 6.7-7.5, pH 6.8-7.5, pH 6.9-7.5, pH 7.0-7.5, pH 7.1-7.5, pH 7.2-7.5, pH 7.3-7.5, or pH 7.4-7.5. In other embodiments, the pH of the vedolizumab composition can be in the range of about pH 6.5-7.5, pH 6.5-7.4, pH 6.5-7.3, pH 6.5-7.2, pH 6.5-7.1, pH 6.5-7.0, pH 6.5-6.9, pH 6.5-6.8, pH 6.5-6.75, or pH 6.5-6.6. In other embodiments, the pH of the vedolizumab composition can be in the range of about 7.0-7.5. In other embodiments, the pH of the vedolizumab composition can be in the range of about 7.5-8.0. In other embodiments, the pH of the vedolizumab composition can be in the range of about 8.0-8.5. In exemplary embodiments, the pH of the vedolizumab composition can be about pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6, pH 8.7, pH 8.8, pH 8.9, or pH 9.0.

A composition comprising vedolizumab at the pH noted above, e.g., a pH at or above 6.5, can be incubated for a period of time sufficient for the percentage of the basic vedolizumab isoform species in the vedolizumab composition to be reduced. In exemplary embodiments, the incubation occurs for a period of 20 minutes or more, 30 minutes or more, 45 minutes or more, 1 hour or more, 1.5 hours or more, 2 hours or more, 3 hours or more, 4 hours or more, 5 hours or more, 8 hours or more, 10 hours or more, 12 hours or more, 15 hours or more, 18 hours or more, 24 hours or more, 48 hours or more, 72 hours or more, 96 hours or more, 120 hours or more, 144 hours or more, or 168 hours or more. In some embodiments, the incubation occurs for a period of about 0.5 days or more, 1 day or more, 2 days or more, 3 days or more, 4 days or more, 5 days or more, 6 days or more, or 7 days or more. In some embodiments, the incubation can take place for about 20 minutes to about 1 hour, about 20 minutes to about 1 hour, about 20 minutes to about 2 hours, about 20 minutes to about 3 hours, about 1 hour to about 3 hours, about 1 hour to about 5 hours, or about 5 hours to about 8 hours. In some embodiments, the incubation can take place for about 8 hours to about 168 hours (7 days) or more. In some embodiments, the incubation can take place for about 8-168 hours, e.g., about 8-144 hours (6 days), about 8-120 hours (5 days), about 8-96 hours (4 days), about 8-72 hours (3 days), about 8-48 hours (2 days), about 8-36 hours, about 8-24 hours (1 day), about 8-18 hours, about 8-12 hours, about 15-36 hours, or about 8-10 hours. In other embodiments, the incubation can take place for about 10 hours to about 168 hours or more, e.g., about 10-168 hours, about 12-168 hours, about 18-168 hours, about 24-168 hours, about 36-168 hours, about 48-168 hours, about 72-168 hours, about 96-168 hours, about 120-168 hours, or about 144-168 hours. In some embodiments, the incubation can take place for about 0.5-7 days, e.g., about 0.5-5 days, about 0.5-4 days, about 0.5-3 days, about 0.5-2 days, or about 0.5-1 day. In some embodiments, the incubation can take place for about 1-5 days, about 1-3 days, or about 1-2 days. In exemplary embodiments, the incubation at or above pH 6.5 occurs for a period of 1-2 days, 1-3 days, or 1-5 days. In other exemplary embodiments, the incubation at or above pH 6.5 occurs for >25% of the total purification time, e.g., for a duration of time beginning with provision of a clarified cell culture harvest to and ending with UF/DF of the purified antibody.

In other embodiments, the incubation occurs for a period of time sufficient to reduce the percentage of basic vedolizumab isoforms in the composition by 1% or more, e.g., 1.5% or more, 2% or more, 2.5% or more, 3% or more, 3.5% or more, 4% or more, 4.5% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 60% or more, 70% or more, 80% or more, 90% or more, 95% or more, 99% or more, or 100% or more. In exemplary embodiments, the incubation occurs for a period of time sufficient to reduce the percentage of basic vedolizumab isoform species in the composition by 1%-5%. In other embodiments, the incubation occurs for a period of time sufficient to reduce the percentage of basic vedolizumab isoform species in the composition by 1-10%. In other embodiments, the incubation occurs for a period of time sufficient to reduce the percentage of basic vedolizumab isoform species in the composition by 2-10%. In other embodiments, the incubation occurs for a period of time sufficient to reduce the percentage of basic vedolizumab isoform species in the composition by 5-10%. In other embodiments, the incubation occurs for a period of time sufficient to reduce the percentage of basic vedolizumab isoform species in the composition by 2-20%. In other embodiments, the incubation occurs for a period of time sufficient to reduce the percentage of basic vedolizumab isoform species in the composition by 5-20%.

In other embodiments, the incubation occurs for a period of time sufficient to produce a low basic species composition comprising an anti-α4β7 antibody (e.g., vedolizumab), wherein the composition has less than less than 16%, less than 15%, less than 14%, less than 13%, less than 12%, less than 11%, less than 10%, or less than 9% total basic isoform species. In some embodiments, the method comprises incubating the composition at a pH of greater than pH 6.3 for a period of time sufficient for the level of basic isoform peak 2 to reach less than 4%, less than 3%, less than 2% or less than 1%. In some embodiments, the method comprises incubating the composition comprising vedolizumab at a pH greater than pH 6.3 (e.g., pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6, pH 8.7, pH 8.8, pH 8.9, or pH 9.0); and incubating the composition comprising the anti-α4β7 antibody (e.g., vedolizumab) for a period of 20 minutes or more, e.g., 30 minutes or more, 1 hour or more, 2 hours or more, 3 hours or more, 4 hours or more, 5 hours or more, 6 hours or more, 7 hours or more, 8 hours or more, 10 hours or more, 12 hours or more, 15 hours or more, 18 hours or more, 24 hours or more, 48 hours or more, 72 hours or more, 96 hours or more, 120 hours or more, 144 hours or more, or 168 hours or more.

During the incubation, in some embodiments, the pH of the vedolizumab composition can be maintained at or above pH 6.3. During the incubation, in other embodiments, the pH of the vedolizumab composition can be maintained at or above pH 6.5. In some embodiments, the vedolizumab composition is maintained at about the same pH for the duration of the incubation period.

The incubation may be performed at any suitable temperature. For example, the incubation may be performed at a temperature in the range of about 0-40° C. In another embodiment, the incubation may be performed at a temperature in the range of about 1-37° C. In some embodiments, the incubation is performed at a temperature in the range of about 0-4° C. or in the range of 4-8° C. In other embodiments, the incubation is performed at ambient temperature. For example, the incubation can be performed at a temperature in the range of about 20-25° C. In other embodiments, the incubation is performed at or about 37° C. In some embodiments, the incubation is performed at a temperature in the range of about 1-25° C. In some embodiments, the incubation is performed at a temperature in the range of about 5-18° C. In some embodiments, the incubation is performed at a temperature in the range of about 15-30° C. In some embodiments, the incubation is performed at a temperature in the range of about 33-37° C. In exemplary embodiments, the incubation is performed at 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

The foregoing methods may optionally comprise an additional step of adjusting the pH of a vedolizumab composition. For example, if it is desirable to reduce the percentage of basic vedolizumab species in a composition comprising vedolizumab, the method may comprise a step of raising the pH of the composition prior to incubation. For example, if the composition comprising vedolizumab is at a pH below 6.5, the method may comprise a step of raising the pH of the composition to a pH at or above 6.5. Conversely, if it is desirable to increase the percentage of basic vedolizumab species in a composition comprising vedolizumab, the method may comprise a step of lowering the pH of the composition prior to incubation. For example, if the composition comprising vedolizumab is at a pH at or above 6.5, the method may comprise a step of lowering the pH of the composition to a pH below 6.5.

The composition comprising vedolizumab can be a liquid solution. In some embodiments, the composition comprising vedolizumab is derived from host cells used to produce vedolizumab, or an antigen binding portion thereof. In some embodiments, the host cells are mammalian cells. In some embodiments, the host cells are Chinese Hamster Ovary (CHO) cells, e.g., CHO cells that lack dihydrofolate reductase (DHFR) expression, or CHO cells that lack glutamine synthetase (GS) expression.

The foregoing methods may be incorporated into small-scale or large-scale processes of vedolizumab purification following isolation of the antibody from cell culture. In certain embodiments, primary recovery of vedolizumab from a host cell culture, e.g., a bioreactor harvest, can employ the steps of centrifugation and filtration. The methods described herein can be performed on the vedolizumab composition prior to or after centrifugation, and/or prior to or after filtration, to reduce the level of basic isoform species in the composition at the corresponding stages of purification. Following primary recovery, downstream process steps that may be used to purify vedolizumab from process-related impurities and/or product-related impurities include, but are not limited to, affinity chromatography (such as Protein A chromatography), depth filtration, cation exchange (CEX), anion exchange (AEX), mixed-mode chromatography (MM), ceramic hydroxyapatite chromatography (CHT), hydrophobic interaction chromatography (HIC), ultrafiltration, and/or diafiltration. The methods described herein can be performed on the vedolizumab composition prior to or following any downstream process steps, to reduce the level of basic isoform species in the composition at the corresponding stage of purification.

For example, the vedolizumab composition can be incubated as described herein prior to or following affinity chromatography. In one embodiment, the method can comprise adjusting the pH of the vedolizumab composition, e.g., the affinity chromatography load material, or the affinity chromatography eluate, to a pH at or above pH 6.5.

In another embodiment, the vedolizumab composition can be incubated as described herein prior to or following depth filtration. In one embodiment, the method can comprise adjusting the pH of the vedolizumab composition prior to or following depth filtration at a pH at or above pH 6.5.

In another embodiment, the vedolizumab composition can be incubated as described herein prior to or following cation exchange (CEX). In one embodiment, the method can comprise adjusting the pH of the vedolizumab composition, e.g., the CEX load material, or the CEX eluate, to a pH at or above pH 6.5.

In another embodiment, the vedolizumab composition can be incubated as described herein prior to or following anion exchange (AEX). In one embodiment, the method can comprise adjusting the pH of the vedolizumab composition, e.g., the AEX load material, or the AEX flow through, to a pH at or above pH 6.5.

In another embodiment, the vedolizumab composition can be incubated as described herein prior to or following mixed-mode chromatography (MM). In one embodiment, the method can comprise adjusting the pH of the vedolizumab composition, e.g., the MM load material, or the MM eluate, to a pH at or above pH 6.5.

In another embodiment, the vedolizumab composition can be incubated as described herein prior to or following ceramic hydroxyapatite chromatography (CHT). In one embodiment, the method can comprise adjusting the pH of the vedolizumab composition, e.g., the CHT load material, or the CHT eluate, to a pH at or above pH 6.5.

In another embodiment, the vedolizumab composition can be incubated as described herein prior to or following hydrophobic interaction chromatography (HIC). In one embodiment, the method can comprise adjusting the pH of the vedolizumab composition, e.g., the HIC load material, or the HIC eluate, to a pH at or above pH 6.5.

In another embodiment, the vedolizumab composition can be incubated as described herein prior to or following ultrafiltration and/or diafiltration (UF/DF). In one embodiment, the method can comprise adjusting the pH of the vedolizumab composition before or after UF/DF to a pH at or above pH 6.5.

### IV. Vedolizumab Compositions Containing Reduced Basic Isoform Species

In some aspects, the invention provides a vedolizumab composition comprising a reduced level of basic vedolizumab isoform species. In some embodiments, the composition having a reduced level of basic isoform species is obtainable by a method provided herein (e.g., see Section III and the Examples). In some embodiments, the composition having a reduced level of basic isoform species is produced by a method provided herein (e.g., see Section III). Accordingly, in one aspect, provided herein is a composition comprising vedolizumab, wherein the composition is produced by a method comprising, inter alia, incubating a composition comprising vedolizumab at a pH greater than pH 6.5 for a time sufficient to reduce the level of basic isoform species in the composition. Following the incubation, the composition comprising vedolizumab can optionally be subjected to further purification steps, designed, for example, to reduce the level process-derived impurities and/or product-derived impurities in the composition.

In another aspect, provided herein is a composition comprising vedolizumab, or an antigen binding portion thereof, wherein the composition is obtainable by the methods described above, e.g., by limiting the duration of time the antibody is exposed to low pH conditions during the purification process.

In some embodiments, provided herein is a composition comprising vedolizumab, or an antigen binding portion thereof, wherein the composition is obtainable by a method comprising, inter alia, (i) providing a clarified cell culture harvest obtained from a culture of recombinant host cells expressing vedolizumab or an antigen binding portion thereof, (ii) and purifying vedolizumab or an antigen binding portion thereof from the cell culture harvest, wherein the antibody or antigen binding portion thereof is exposed to a pH at or below 3.5 (e.g., pH 2.5-3.5, pH below 3.0, or pH below 3.5) for no more than 20 minutes, no more than 30 minutes, no more than 45 minutes, no more than 1 hour, no more than 3 hours, no more than 5 hours, no more than 7 hours, no more than 10 hours, or no more than 12 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 3.5 (e.g., pH 2.5-3.5, pH below 3.0, or pH below 3.5) for a longer duration of time, i.e., greater than 20 minutes, greater than 30 minutes, greater than 45 minutes, greater than 1 hour, greater than 3 hours, greater than 5 hours, greater than 7 hours, greater than 10 hours, or greater than 12 hours.

In some embodiments, provided herein is a composition comprising vedolizumab, or an antigen binding portion thereof, wherein the composition is obtainable by a method comprising, inter alia, (i) providing a clarified cell culture harvest obtained from a culture of recombinant host cells expressing vedolizumab or an antigen binding portion thereof, (ii) and purifying vedolizumab or an antigen binding portion thereof from the cell culture harvest, wherein the antibody or antigen binding portion thereof is exposed to a pH at or below 4.0 (e.g., pH 3.6 to 4.0), for no more than 20 minutes, no more than 30 minutes, no more than 45 minutes, no more than 1 hour, no more than 3 hours, no more than 5 hours, no more than 10 hours, no more than 12 hours, no more than 15 hours, no more than 18 hours, or no more than 24 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 4.0 (e.g., pH 3.6-4.0) for a longer duration of time, i.e., greater than 20 minutes, greater than 30 minutes, greater than 45 minutes, greater than 1 hour, greater than 3 hours, greater than 5 hours, greater than 10 hours, greater than 12 hours, greater than 15 hours, greater than 18 hours, or greater than 24 hours.

In another aspect, provided herein is a composition comprising vedolizumab, or an antigen binding portion thereof, wherein the composition is obtainable by a method comprising, inter alia, (i) providing a clarified cell culture harvest obtained from a culture of recombinant host cells expressing vedolizumab or an antigen binding portion thereof, (ii) and purifying vedolizumab or an antigen binding portion thereof from the cell culture harvest, wherein the antibody or antigen binding portion thereof is exposed to a pH at or below 4.5 (e.g., pH 4.1-4.5) for no more than 3 hours, no more than 5 hours, no more than 10 hours, or no more than 12 hours, no more than 18 hours, no more than 24 hours, no more than 36 hours, no more than 48 hours, no more than 72 hours, or no more than 96 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 4.5 (e.g., pH 4.1-4.5) for a longer duration of time, i.e., greater than 3 hours, greater than 5 hours, greater than 10 hours, greater than 12 hours, greater than 18 hours, greater than 24 hours, greater than 36 hours, greater than 48 hours, greater than 72 hours, or greater than 96 hours.

In another aspect, provided herein is a composition comprising vedolizumab, or an antigen binding portion thereof, wherein the composition is obtainable by a method comprising, inter alia, (i) providing a clarified cell culture harvest obtained from a culture of recombinant host cells expressing vedolizumab or an antigen binding portion thereof, (ii) and purifying vedolizumab or an antigen binding portion thereof from the cell culture harvest, wherein the antibody or antigen binding portion thereof is exposed to a pH at or below 5.0 (e.g., pH 4.6-5.0) for no more than 3 hours, no more than 5 hours, no more than 10 hours, or no more than 12 hours, no more than 18 hours, no more than 24 hours, no more than 36 hours, no more than 48 hours, no more than 72 hours, or no more than 96 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 5.0 (e.g., pH 4.6-5.0) for a longer duration of time, i.e., greater than 3 hours, greater than 5 hours, greater than 10 hours, greater than 12 hours, greater than 18 hours, greater than 24 hours, greater than 36 hours, greater than 48 hours, greater than 72 hours, or greater than 96 hours.

In another aspect, provided herein is a composition comprising vedolizumab, or an antigen binding portion thereof, wherein the composition is obtainable by a method comprising, inter alia, (i) providing a clarified cell culture harvest obtained from a culture of recombinant host cells expressing vedolizumab or an antigen binding portion thereof, (ii) and purifying vedolizumab or an antigen binding portion thereof from the cell culture harvest, wherein the antibody or antigen binding portion thereof is exposed to a pH at or below 5.5 (e.g., pH 5.1-5.5) for no more than 3 hours, no more than 5 hours, no more than 10 hours, or no more than 12 hours, no more than 18 hours, no more than 24 hours, no more than 36 hours, no more than 48 hours, no more than 72 hours, or no more than 96 hours, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 5.5 (e.g., pH 5.1-5.5) for a longer duration of time, i.e., greater than 3 hours, greater than 5 hours, greater than 10 hours, greater than 12 hours, greater than 18 hours, greater than 24 hours, greater than 36 hours, greater than 48 hours, greater than 72 hours, or greater than 96 hours.

In some embodiments of the foregoing aspects, the antibody or antigen binding portion thereof comprises a heavy chain variable region comprising the amino acid sequence set forth in SEQ ID NO:1, and a light chain variable region comprising the amino acid sequence set forth in SEQ ID NO:5.

In some embodiments of the foregoing aspects, the step of purifying the anti-α4β7 antibody or an antigen binding portion thereof comprises one or more of Protein A chromatography, anion exchange chromatography, cation exchange chromatography, mixed mode chromatography, hydrophobic interaction chromatography, and combinations thereof.

In some embodiments of the foregoing aspects, the host cells expressing the anti-α4β7 antibody or an antigen binding portion thereof are GS-CHO cells. In other embodiments, the host cells are DHFR-CHO cells.

In some embodiments of the foregoing aspects, the composition comprises less than 16%, less than 15%, less than 14%, less than 13%, less than 12%, less than 11%, less than 10%, or less than 9% basic isoform species.

In some embodiments of the foregoing aspects, the composition comprises less than 4%, less than 3%, less than 2%, or less than 1% basic isoform peak 2.

In some embodiments, provided herein is a vedolizumab composition wherein basic species of vedolizumab comprise 15% or less of the vedolizumab isoforms in the composition. For example, in some embodiments, provided herein is a vedolizumab composition comprising basic isoform species at a level of about 14% or less, 13% or less, 12% or less, 11% or less, 10% or less, 9% or less, 8% or less, 7% or less, 6% or less, 5% or less, 4% or less, 3% or less, 2% or less, or 1% or less.

In some embodiments, the level of basic isoform species in the vedolizumab composition is about 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, or 1% to 2%. In other embodiments, the level of basic isoform species in the vedolizumab composition is about 2% to 11%, 3% to 11%, 4% to 11%, 5% to 11%, 6% to 11%, 7% to 11%, 8% to 11%, 9% to 11%, or 10% to 11%. In other embodiments, the level of basic isoform species in the vedolizumab composition is about 1% to 10%, 2% to 10%, 3% to 10%, 4% to 10%, 5% to 10%, 6% to 10%, 7% to 10%, 8% to 10%, or 9% to 10%. In other embodiments, the level of basic isoform species in the vedolizumab composition is about 1% to 9%, 2% to 9%, 3% to 9%, 4% to 9%, 5% to 9%, 6% to 9%, 7% to 9%, or 8% to 9%. In other embodiments, the level of basic isoform species in the vedolizumab composition is about 1% to 8%, 2% to 8%, 3% to 8%, 4% to 8%, 5% to 8%, 6% to 8%, or 7% to 8%. In other embodiments, the level of basic isoform species in the vedolizumab composition is about 1% to 7%, 2% to 7%, 3% to 7%, 4% to 7%, 5% to 7%, or 6% to 7%. In other embodiments, the level of basic isoform species in the vedolizumab composition is about 1% to 6%, 2% to 6%, 3% to 6%, 4% to 6%, or 5% to 6%. In other embodiments, the level of basic isoform species in the vedolizumab composition is about 1% to 5%, 2% to 5%, 3% to 5%, or 4% to 5%. In an exemplary embodiment, the level of basic isoform species in the vedolizumab composition is about 5% or less.

In some embodiments, the percentage of basic isoform species in the vedolizumab composition is reduced by about 1% or more, e.g., 1.5% or more, 2% or more, 2.5% or more, 3% or more, 3.5% or more, 4% or more, 4.5% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 60% or more, 70% or more, 80% or more, 90% or more, 95% or more, 99% or more, or 100% or more, relative to the percentage of basic isoform species in a vedolizumab composition produced by the same method, without incubation at a pH greater than pH 6.5, as described herein.

The percentage of basic isoform species in a composition comprising vedolizumab can be determined by any suitable method, including but not limited to CEX-HPLC. In some embodiments, provided herein is a low basic species composition comprising an anti-α4β7 antibody, or an antigen binding portion thereof (e.g., vedolizumab), wherein the composition comprises less than 16%, less than 15%, less than 14%, less than 13%, less than 12%, less than 11%, less than 10%, or less than 9% total basic isoform species of the anti-α4β7 antibody, or an antigen binding portion thereof, wherein the basic isoform species have a net positive charge relative to a main isoform of the anti-α4β7 antibody, or an antigen binding portion thereof, as determined by CEX, wherein the anti-α4β7 antibody, or an antigen binding portion thereof, comprises a heavy chain variable region comprising SEQ ID NO:1, and a light chain variable region comprising SEQ ID NO:5. In some embodiments, the composition comprises a first basic isoform peak (BP1) and a second basic isoform peak (BP2). In some such embodiments, the composition comprises less than 2% BP2, less than 1.5% BP2, less than 1% BP2, or less than 0.7% BP2. In some embodiments, the composition comprises 1.5% to 2.5% BP2, 1.2% to 2.2%, 1% to 2% BP2, 1% to 1.8% BP2, 1% to 1.6% BP2, 1% to 1.5% BP2, 0.8% to 1.8% BP2, 0.8% to 1.6% BP2, 0.8% to 1.4% BP2, 0.8% to 1.2% BP2, 0.8% to 1% BP2, 0.7% to 1.7% BP2, 0.7% to 1.5% BP2, 0.7% to 1.3% BP2, 0.7% to 1% BP2, 0.6% to 1.6% BP2, 0.6% to 1.4% BP2, 0.6% to 1.2% BP2, 0.6% to 1% BP2, 0.6% to 0.8% BP2. 0.5% to 1.5% BP2, 0.5% to 1.3% BP2, 0.5% to 1% BP2, or 0.5% to 0.8% BP2.

In some embodiments, the ratio of BP1 to BP2 in the composition is at least 3 (e.g., at least 5, at least 6, at least 7, at least 8, at least 9, or at least 10). In some embodiments, the ratio of BP1 to BP2 is from 2 to 12, 2 to 10, 2 to 8, 2 to 6, 2 to 4, 3 to 12, 3 to 11, 3 to 9, 3 to 6, 3 to 5, 3 to 4, 4 to 12, 4 to 11, 4 to 10, 4 to 8, 4 to 6, 4 to 5, 5 to 12, 5 to 11, 5 to 10, 5 to 9, 5 to 8, 5 to 6, 6 to 12, 6 to 11, 6 to 10, 6 to 8, 6 to 7, 7 to 12, 7 to 11, 7 to 10, 7 to 9, 7 to 8, 8 to 12, 8 to 11, 8 to 10, 8 to 9, 9 to 12, 9 to 11, or 9 to 10.

In some embodiments, the low basic species composition comprises less than 8% (e.g., less than 7%, less than 6%, or less than 5%) total basic isoform species of the anti-α4β7 antibody (e.g., vedolizumab). In some embodiments, the low basic species composition comprises 4% to 8%, 4% to 7.5% 4% to 7%, 4% to 6.5%, 4% to 6%, 4% to 5.5%, 4% to 5%, 5% to 8%, 5% to 7.5% 5% to 7%, 5% to 6.5%, 5% to 6%, 6% to 8%, 6% to 7.5%, or 6% to 7% total basic isoform species of the anti-α4β7 antibody (e.g., vedolizumab).

In some embodiments, the foregoing compositions can be incorporated into a pharmaceutical composition comprising an anti-α4β7 antibody, or an antigen binding portion thereof (e.g., vedolizumab), and a pharmaceutically acceptable carrier or excipient. Accordingly, in some embodiments, provided herein is a pharmaceutical composition comprising a low basic species anti-α4β7 antibody, or an antigen binding portion thereof (e.g., vedolizumab), and a pharmaceutically acceptable carrier. The antibody formulation may remain as a liquid or be lyophilized into a dry antibody formulation. In one aspect, the dry, lyophilized antibody formulation is provided in a single dose vial comprising 150 mg, 180 mg, 240 mg, 300 mg, 360 mg, 450 mg or 600 mg of anti-α4β7 antibody and can be reconstituted with a liquid, such as sterile water, for administration. In another aspect, the anti-α4β7 antibody, e.g., vedolizumab, is in a stable liquid pharmaceutical composition stored in a container, e.g., a vial, a syringe or cartridge, at about 2-8° C. until it is administered to a subject in need thereof. In some embodiments, the syringe or cartridge can provide a single dose of 54 mg, 108 mg, 160 mg, or 216 mg of the antibody. In some embodiments, the reconstituted lyophilized formulation or the stable liquid pharmaceutical composition of anti-α4β7 antibody can comprise one or more excipients, including but not limited to an amino acid (e.g., arginine, histidine, and/or histidine monohydrochloride), a sugar (e.g., sucrose), a surfactant (e.g., polysorbate 80), and/or a buffer (e.g., citrate, phosphate, etc.). In one embodiment, the reconstituted lyophilized formulation or the stable liquid pharmaceutical composition of anti-α4β7 antibody comprises L-arginine, L-histidine, L-histidine monohydrochloride, sucrose, and/or polysorbate 80. In another embodiment, the reconstituted lyophilized formulation or the stable liquid pharmaceutical composition of anti-α4β7 antibody comprises citrate, arginine, histidine, and/or polysorbate 80. Additional formulations and uses of an anti-α4β7 antibody are described, for example, in U.S. Pat. Nos. 9,764,033 and 10,040,855. The entire contents of each of the foregoing patents are incorporated herein by reference.

### V. Preparation of Vedolizumab Compositions Containing Reduced Levels of Host Cell Protein

In some aspects, the invention provides methods of producing a composition comprising vedolizumab having a reduced amount of host cell protein. This method is based on the surprising finding that using an AEX buffer (e.g., loading buffer) with reduced conductivity relative to standard operating conditions yields a vedolizumab composition having a reduced level of host cell proteins (HCPs), relative to vedolizumab compositions produced under standard operating conditions.

Accordingly, in one aspect, the invention provides a method of producing a composition comprising vedolizumab having a reduced amount of host cell protein (HCP), wherein the method involves contacting a sample containing vedolizumab and HCP with an anion exchange resin in the presence of a loading buffer, wherein the loading buffer has a reduced conductivity relative to standard buffer conditions, and collecting a flow through material from the anion exchange resin, wherein the flow through comprises vedolizumab and a reduced amount of HCP. In exemplary embodiments, AEX is performed in flow-through mode, where vedolizumab does not bind the AEX resin, and is collected in the flow through material without a separate elution step.

The standard operating range of buffer conductivity for anion exchange (AEX) (e.g., via an anion exchange Q membrane adsorber), is approximately 11-15 mS/cm (average approximately 13.6 mS/cm). Accordingly, in some embodiments, the standard AEX buffer conditions comprise a loading buffer having a conductivity of about 11 mS/cm or greater, about 12 mS/cm or greater, about 13 mS/cm or greater, about 14 mS/cm or greater, or about 15 mS/cm or greater. In some embodiments, the standard buffer conductivity is about 11 mS/cm to about 12 mS/cm, about 11 mS/cm to about 13 mS/cm, about 11 mS/cm to about 14 mS/cm, or about 11 mS/cm to about 15 mS/cm. In some embodiments, the standard buffer conductivity is about 14 mS/cm to about 15 mS/cm, about 13 mS/cm to about 15 mS/cm, about 12 mS/cm to about 15 mS/cm, or about 11 mS/cm to about 15 mS/cm. In some embodiments, the standard buffer conductivity is about 11 mS/cm, about 12 mS/cm, about 13 mS/cm, about 14 mS/cm, or about 15 mS/cm.

The reduced conductivity AEX buffer (e.g., AEX loading buffer) used in the methods described herein has a reduced conductivity relative to a standard AEX buffer condition. For example, in certain embodiments, the loading buffer with reduced conductivity has a conductivity of about 15 mS/cm or less, about 14 mS/cm or less, about 13 mS/cm or less, about 12 mS/cm or less, about 11 mS/cm or less, about 10 mS/cm or less, about 9 mS/cm or less, about 8 mS/cm or less, about 7 mS/cm or less, about 6 mS/cm or less, about 5 mS/cm or less, about 4 mS/cm or less, about 3 mS/cm or less, or about 2 mS/cm or less. In some embodiments, the loading buffer having a reduced conductivity has a conductivity of about 11 mS/cm or less.

In certain embodiments, the loading buffer with reduced conductivity has a conductivity of about 1 mS/cm to about 11 mS/cm, about 2 mS/cm to about 11 mS/cm, about 3 mS/cm to about 11 mS/cm, about 4 mS/cm to about 11 mS/cm, about 5 mS/cm to about 11 mS/cm, about 6 mS/cm to about 11 mS/cm, about 7 mS/cm to about 11 mS/cm, about 8 mS/cm to about 11 mS/cm, about 9 mS/cm to about 11 mS/cm, or about 10 mS/cm to about 11 mS/cm, including ranges within one or more of the preceding. In some embodiments, the loading buffer having reduced conductivity has a conductivity of about 11 mS/cm to about 12 mS/cm, about 11 mS/cm to about 13 mS/cm, about 11 mS/cm to about 14 mS/cm, or about 11 mS/cm to about 14.5 mS/cm, including ranges within one or more of the preceding. In certain embodiments, the loading buffer with reduced conductivity has a conductivity of about 1 mS/cm to about 2 mS/cm, about 1 mS/cm to about 3 mS/cm, about 1 mS/cm to about 4 mS/cm, about 1 mS/cm to about 5 mS/cm, about 1 mS/cm to about 6 mS/cm, about 1 mS/cm to about 7 mS/cm, about 1 mS/cm to about 8 mS/cm, about 1 mS/cm to about 9 mS/cm, about 1 mS/cm to about 10 mS/cm, or about 1 mS/cm to about 11 mS/cm, including ranges within one or more of the preceding.

In certain embodiments, the loading buffer with reduced conductivity has a conductivity of about 1 mS/cm, about 1.5 mS/cm, about 2 mS/cm, about 2.5 mS/cm, about 3 mS/cm, about 3.5 mS/cm, about 4 mS/cm, about 4.5 mS/cm, about 5 mS/cm, about 5.5 mS/cm, about 6 mS/cm, about 6.5 mS/cm, about 7 mS/cm, about 7.5 mS/cm, about 8 mS/cm, about 8.5 mS/cm, 9 mS/cm, 9.5 mS/cm, 10 mS/cm, 10.5 mS/cm, 11 mS/cm, 11.5 mS/cm, 12 mS/cm, 12.5 mS/cm, 13 mS/cm, 13.5 mS/cm, 14 mS/cm, 14.5 mS/cm, or 15 mS/cm.

In some embodiments, the method can further comprise applying a wash buffer to the AEX resin following application of the loading buffer containing vedolizumab. In some embodiments, the wash buffer has the same conductivity as the loading buffer. In other embodiments, the wash buffer has an increased conductivity relative to the loading buffer. In other embodiments, the wash buffer has a decreased conductivity relative to the loading buffer.

In some embodiments, the loading buffer comprises sodium chloride and/or sodium phosphate. In exemplary embodiments, the loading buffer contains 40-70 mM NaCl (e.g., 40 mM, 45 mM, 50 mM, 55 mM, 60 mM, 65 mM or 70 mM NaCl). In some embodiments, the loading buffer contains 55-65 mM NaCl. In addition, or alternatively, the loading buffer can contain sodium phosphate. In exemplary embodiments, the loading buffer contains 20-50 mM sodium phosphate (e.g., 20 mM, 25 mM, 30 mM, 35 mM, 40 mM, 45 mM, or 50 mM sodium phosphate). In some embodiments, the loading buffer contains 35-45 mM sodium phosphate. In some embodiments, the loading buffer is at a pH at or above pH 6.5, e.g., pH 6.5-8.5, pH 7.0-7.5, pH 6.8-7.4, etc. In some embodiments, the loading buffer is at a pH of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

In some embodiments, provided herein is a method of purifying an α4β7 antibody, e.g., vedolizumab, comprising contacting a solution comprising the antibody with an AEX resin equilibrated in a low conductivity buffer in flow through mode, and collecting the flow through material. In one embodiment, the low conductivity solution comprises a mixture of NaCl and a buffer at a pH of 6.5-8.5. In some embodiments, the low conductivity solution has a conductivity of 5 to 15 mS/cm, 5 to 11 mS/cm, 7 to 10 mS/cm or about 10 mS/cm.

In some embodiments, the anion exchange resin is formatted as an anion exchange membrane. In some embodiments, the anion exchange resin is formatted as an anion exchange chromatography column. In some embodiments, the anion exchange resin comprises a quaternary amine functional group. Exemplary AEX resins include, but are not limited to, Mustang Q (Pall Corporation, Port Washington, N.Y.), Sartobind Q (Sartorius GmbH, Goettingen, Germany). Other exemplary AEX resins include, for example, Eshmuno Q resin (EMD Millipore, Burlington, Mass.) and Nuvia Q resin (Bio-Rad, Hercules, Calif.).

The HCP can be derived from host cells used to produce vedolizumab, or an antigen binding portion thereof. For example, in some embodiments, vedolizumab is produced in Chinese Hamster Ovary (CHO) cells, and the HCP is a CHO cell protein. In certain embodiments, the HCP originates from a CHO cell that lacks dihydrofolate reductase (DHFR) expression. In certain embodiments, the HCP originates from a CHO cell that lacks glutamine synthetase (GS) expression.

In certain embodiments, the amount of HCP in the flow through is about 8 ppm or less, about 7.5 ppm or less, about 7 ppm or less, about 6.5 ppm or less, about 6 ppm or less, about 5.5 ppm or less, about 5 ppm or less, about 4.5 ppm or less, about 4 ppm or less, about 3.5 ppm or less, about 3 ppm or less, about 2.5 ppm or less, about 2 ppm or less, about 1.5 ppm or less, or about 1 ppm or less.

In some embodiments, the amount of HCP in the flow through is 1 ppm to 8 ppm, 2 ppm to 8 ppm, 3 ppm to 8 ppm, 4 ppm to 8 ppm, 5 ppm to 8 ppm, 6 ppm to 8 ppm, or 7 ppm to 8 ppm, including ranges within one or more of the preceding. In some embodiments, the amount of HCP in the flow through is 1 ppm to 2 ppm, 1 ppm to 3 ppm, 1 ppm to 4 ppm, 1 ppm to 5 ppm, 1 ppm to 6 ppm, 1 ppm to 7 ppm, or 1 ppm to 8 ppm, including ranges within one or more of the preceding. In some embodiments, the amount of HCP in the flow through is 1 ppm to 3 ppm, 3 ppm to 5 ppm, or 5 ppm to 7 ppm.

In certain embodiments, the amount of HCP in the flow through is reduced by at least about 0.5%, at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98% or more than about 98% relative to the amount of HCP in a flow through material produced when the method is performed using the same sample with a loading buffer having standard conductivity (e.g., conductivity at or above 11-15 mS/cm).

In certain embodiments, the amount of HCP in the flow through is reduced by about 0.5% to about 50%, about 1% to about 50%, about 2% to about 50%, about 5% to about 50%, about 10% to about 50%, about 15% to about 50%, about 20% to about 50%, about 25% to about 50%, about 30% to about 50%, about 35% to about 50%, about 40% to about 50%, or about 45% about to about 50%. In certain embodiments, the amount of HCP in the flow through is reduced by about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, or about 50% to about 98% relative to the amount of HCP in the flow through produced when the method is performed using the same sample with a loading buffer having standard conductivity (e.g., approximately 11-15 mS/cm).

In certain embodiments, the amount of HCP in the flow through is reduced by about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 98%. In certain embodiments, the amount of HCP in the flow through is reduced by about 0.5% to about 1%, about 1% to about 2%, about 2% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% about to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 98% relative to the amount of HCP in the flow through produced when the method is performed using the same sample with a loading buffer having standard conductivity (e.g., approximately 11-15 mS/cm).

The sample containing vedolizumab and HCP is derived from a mammalian cell culture, optionally following one or more additional purification steps, including, for example, affinity chromatography, cation exchange chromatography, hydrophobic interaction chromatography, ceramic hydroxyapatite (CHT) chromatography, and mixed-mode chromatography, or a combination thereof. In addition, following the AEX methods described herein, the level of HCP in a sample containing vedolizumab can optionally be further reduced by one or more additional purification steps, including, for example, affinity chromatography, cation exchange chromatography, hydrophobic interaction chromatography, ceramic hydroxyapatite (CHT) chromatography, and mixed-mode chromatography, or a combination thereof.

Accordingly, the methods described herein can include, in some embodiments, one or more additional purification steps to further reduce the level of HCP in a sample containing vedolizumab. In one embodiment, the invention provides methods of reducing the level of HCP in a composition comprising vedolizumab, using the AEX methods described herein, and further comprising one or more additional purification steps. The one or more additional purification steps may be performed before or after the AEX methods described herein. In some embodiments, the one or more additional purification steps include one or more chromatographic separations. In exemplary embodiments, the one or more additional purifications steps include affinity chromatography (e.g., Protein A chromatography), cation exchange chromatography, hydrophobic interaction chromatography, ceramic hydroxyapatite (CHT) chromatography, or mixed-mode chromatography, or a combination thereof.

In an exemplary embodiment, the invention provides a method of reducing the level of HCP in a composition comprising vedolizumab, that comprises providing a composition comprising vedolizumab and HCP, purifying vedolizumab from the HCP by performing affinity chromatography, mixed-mode chromatography, and/or cation exchange chromatography, and further purifying vedolizumab from the HCP by performing the AEX methods described herein. In one embodiment, the load material for the AEX methods described herein comprises a cation exchange eluate. The foregoing method can be used to produce a composition comprising vedolizumab having a reduced level of HCP, relative to the level of HCP present in a composition resulting from performance of the same method, using an anion exchange buffer at a conductivity at or above 11-15 mS/cm.

In another exemplary embodiment, the invention provides a method of reducing the level of HCP in a composition comprising vedolizumab, that comprises providing a composition comprising vedolizumab and HCP, purifying vedolizumab from the HCP by performing affinity chromatography, cation exchange chromatography, and/or hydroxyapatite chromatography (e.g., ceramic hydroxyapatite (CHT) chromatography), and further purifying vedolizumab from the HCP by performing the AEX methods described herein. In one embodiment, the load material for the AEX methods described herein comprises a hydroxyapatite chromatography eluate. The foregoing method can be used to produce a composition comprising vedolizumab having a reduced level of HCP, relative to the level of HCP present in a composition resulting from performance of the same method, using an anion exchange buffer at a conductivity at or above 11-15 mS/cm.

Vedolizumab content and/or host cell protein content can be measured by any methods known in the art, including but not limited to HCP ELISA, chromatography (e.g., SEC), analytical ultracentrifugation, light scattering (DLS or MALLS), mass spectrometry (e.g., MALDI-TOF MS), or nanoscale measurement, such as nanoparticle tracking analysis NTA, NanoSight Ltd, Wiltshire, UK).

In some embodiments, the method further comprises processing the AEX flow through material to exchange the elution buffer by a process comprising ultrafiltration and/or diafiltration, to a buffer comprising one or more pharmaceutically acceptable carriers or excipients.

### VI. Vedolizumab Compositions Containing Reduced Host Cell Protein

In some aspects, the invention provides vedolizumab compositions comprising reduced host cell protein. In some embodiments, the composition having reduced host cell protein is produced by a method provided herein (e.g., see Section V). Accordingly, in one aspect, provided herein is a composition comprising vedolizumab, wherein the composition is produced by contacting a sample containing vedolizumab and HCP with an anion exchange resin in the presence of a loading buffer, wherein the loading buffer has a reduced conductivity relative to standard buffer conditions, and collecting the flow through material from the anion exchange resin, wherein the flow through material comprises vedolizumab and a reduced amount of HCP. Following elution from the AEX resin, the flow through material can optionally be processed to exchange the elution buffer to a buffer comprising one or more pharmaceutically acceptable carriers or excipients, thereby forming a pharmaceutical composition having a reduced amount of HCP. This buffer exchange step may be performed using standard methods, including, for example, ultrafiltration and/or diafiltration.

The HCP can be derived from host cells used to produce vedolizumab, or an antigen binding portion thereof. For example, in some embodiments, vedolizumab is produced in Chinese Hamster Ovary (CHO) cells, and the HCP is a CHO cell protein. In certain embodiments, the HCP originates from a CHO cell that lacks dihydrofolate reductase (DHFR) expression. In certain embodiments, the HCP originates from a CHO cell that lacks glutamine synthetase (GS) expression.

In certain embodiments, the amount of HCP in the composition is about 8 ppm or less, about 7.5 ppm or less, about 7 ppm or less, about 6.5 ppm or less, about 6 ppm or less, about 5.5 ppm or less, about 5 ppm or less, about 4.5 ppm or less, about 4 ppm or less, about 3.5 ppm or less, about 3 ppm or less, about 2.5 ppm or less, about 2 ppm or less, about 1.5 ppm or less, or about 1 ppm or less.

In some embodiments, the amount of HCP in the composition is 1 ppm to 8 ppm, 2 ppm to 8 ppm, 3 ppm to 8 ppm, 4 ppm to 8 ppm, 5 ppm to 8 ppm, 6 ppm to 8 ppm, or 7 ppm to 8 ppm, including ranges within one or more of the preceding. In some embodiments, the amount of HCP in the composition is 1 ppm to 2 ppm, 1 ppm to 3 ppm, 1 ppm to 4 ppm, 1 ppm to 5 ppm, 1 ppm to 6 ppm, 1 ppm to 7 ppm, or 1 ppm to 8 ppm, including ranges within one or more of the preceding. In some embodiments, the amount of HCP in the composition is 1 ppm to 3 ppm, 3 ppm to 5 ppm, or 5 ppm to 7 ppm.

In certain embodiments, the amount of HCP in the composition is reduced by at least about 0.5%, at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98% or more than about 98% relative to the amount of HCP in a composition produced by a method performed using the same sample with a loading buffer having standard conductivity (e.g., conductivity at or above 11-15 mS/cm).

In certain embodiments, the amount of HCP in the composition is reduced by about 0.5% to about 50%, about 1% to about 50%, about 2% to about 50%, about 5% to about 50%, about 10% to about 50%, about 15% to about 50%, about 20% to about 50%, about 25% to about 50%, about 30% to about 50%, about 35% to about 50%, about 40% to about 50%, or about 45% about to about 50%. In certain embodiments, the amount of HCP in the flow through material is reduced by about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, or about 50% to about 98% relative to the amount of HCP in a composition produced by a method performed using the same sample with a loading buffer having standard conductivity (e.g., conductivity at or above 11-15 mS/cm).

In certain embodiments, the amount of HCP in the composition is reduced by about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 98%. In certain embodiments, the amount of HCP in the flow through material is reduced by about 0.5% to about 1%, about 1% to about 2%, about 2% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% about to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 98% relative to the amount of HCP in a composition produced by a method performed using the same sample with a loading buffer having standard conductivity (e.g., conductivity at or above 11-15 mS/cm).

Vedolizumab content and/or host cell protein content can be measured by any methods known in the art, including but not limited to HCP ELISA, chromatography (e.g., SEC), analytical ultracentrifugation, light scattering (DLS or MALLS), mass spectrometry (e.g., MALDI-TOF MS), or nanoscale measurement, such as nanoparticle tracking analysis NTA, NanoSight Ltd, Wiltshire, UK).

### VII. Preparation of Vedolizumab Using Mixed Mode Chromatography

Described herein are buffers that maximize the yield of vedolizumab following elution from a mixed mode chromatography resin. Protein loss can occur during the wash step of mixed mode purification using, for example, a ceramic hydroxyapatite resin, under high concentration loading conditions (e.g., a loading concentration of at least 14 g/L, 15 g/L, 17 g/L, 10 g/L, 25 g/L, 30 g/L, 35 g/L or more). To increase the loading capacity of a mixed mode resin, e.g., a ceramic hydroxyapatite resin, and improve the resulting yield of vedolizumab, the equilibration, loading, and wash buffers can be optimized to reduce loss of the antibody when washing the column. Surprisingly, the method described herein allows for a greater load concentration on a mixed mode resin, and increases the yield by 2-3% relative to standard mixed mode buffers, without altering product quality (e.g., percent aggregates) in the final eluate.

Accordingly, in one aspect, the invention provides a method of increasing the yield of vedolizumab recovered following elution from a mixed mode chromatography resin, comprising equilibrating the mixed mode chromatography resin with an equilibration buffer, loading a solution comprising vedolizumab and a loading buffer onto the mixed mode chromatography resin such that vedolizumab binds the mixed mode chromatography resin, washing the mixed mode chromatography resin with a wash buffer, and eluting vedolizumab from the mixed mode chromatography resin with an elution buffer, wherein the equilibration buffer, the loading buffer, and/or the wash buffer have a pH at or below 7.0. Standard buffers for mixed mode chromatography can be at a higher pH, i.e., a pH of 7.2 or above. In some embodiments of the present invention, an equilibration buffer is used that has pH at or below 7.0. In some embodiments, a loading buffer is used that has a pH at or below 7.0. In some embodiments, a wash buffer is used that has a pH at or below 7.0.

A range of buffer pH below 7.0 can be used to prevent loss of vedolizumab during the wash step of mixed mode purification. For example, in some embodiments, the equilibration buffer, the loading buffer, and/or the wash buffer can have a pH of less than 7.0, less than 6.9, less than 6.8, less than 6.7, less than 6.6, less than 6.5, less than 6.4, less than 6.3, less than 6.2, less than 6.1, less than 6.0, less than 5.9, less than 5.8, less than 5.7, less than 5.6, or less than 5.5. For example, the buffer can have a pH of about 7.0, 6.9, 6.8, 6.7, 6.6, 6.5, 6.4, 6.3, 6.2, 6.1, 6.0, 5.9, 5.8, 5.7, 5.6, or 5.5. In some embodiments, the buffer has a pH in the range of about 7.0-5.5. In other embodiments, the buffer has a pH in the range of about 7.0-6.0. In other embodiments, the buffer has a pH in the range of about 7.0-6.5. In other embodiments, the buffer has a pH in the range of about 6.8-5.8. In other embodiments, the buffer has a pH in the range of about 6.8-6.0. In other embodiments, the buffer has a pH in the range of about 6.8-6.5. In other embodiments, the buffer has a pH of about 6.6-6.8.

In addition, or alternatively, the buffer used for equilibration, loading, and/or washing of a mixed mode chromatography resin for purification of vedolizumab can have a total salt concentration of less than 70 mM. For example, the buffer can have a salt concentration of less than 70 mM, less than 65 mM, less than 60 mM, less than 55 mM, less than 50 mM, less than 45 mM, less than 40 mM, less than 35 mM, or less than 30 mM. In some embodiments, the buffer has a salt concentration of about 70 mM, about 65 mM, about 60 mM, about 55 mM, about 50 mM, about 45 mM, about 40 mM, about 35 mM, or about 30 mM. In other embodiments, the buffer has a salt concentration in the range of 30-70 mM. In some embodiments, the buffer has a salt concentration in the range of 40-65 mM. In some embodiments, the buffer has a salt concentration in the range of 45-65 mM. In some embodiments, the buffer has a salt concentration in the range of 50-60 mM. In some embodiments, the buffer has a salt concentration in the range of 40-50 mM. In some embodiments, the buffer has a salt concentration in the range of 45-55 mM. In some embodiments, the salt present in the buffer used for equilibration, loading, and/or washing of a CHT chromatography resin for purification of vedolizumab comprises sodium chloride and/or sodium phosphate.

In some embodiments, the buffer used for equilibration, loading, and/or washing of a mixed mode chromatography resin for purification of vedolizumab can have a sodium chloride concentration of less than 70 mM. For example, the buffer can have a sodium chloride concentration of less than 70 mM, less than 65 mM, less than 60 mM, less than 55 mM, less than 50 mM, less than 45 mM, less than 40 mM, less than 35 mM, or less than 30 mM. In some embodiments, the buffer has a sodium chloride concentration of about 70 mM, about 65 mM, about 60 mM, about 55 mM, about 50 mM, about 45 mM, about 40 mM, about 35 mM, or about 30 mM. In other embodiments, the buffer has a sodium chloride concentration in the range of 30-70 mM. In some embodiments, the buffer has a sodium chloride concentration in the range of 40-65 mM. In some embodiments, the buffer has a sodium chloride concentration in the range of 45-65 mM. In some embodiments, the buffer has a sodium chloride concentration in the range of 50-60 mM. In some embodiments, the buffer has a sodium chloride concentration in the range of 40-50 mM. In some embodiments, the buffer has a sodium chloride concentration in the range of 45-55 mM. In some embodiments, the foregoing sodium chloride buffers can further comprise sodium phosphate. In some embodiments, the foregoing sodium chloride buffers can further comprise 1-30 mM sodium phosphate, e.g., 5-20 mM sodium phosphate, 10-20 mM sodium phosphate, etc. In exemplary embodiments, the sodium chloride buffers can further comprise sodium phosphate at a concentration of 1 mM, 2 mM, 3 mM, 4 mM, 5 mM, 6 mM, 7 mM, 8 mM, 9 mM, 10 mM, 15 mM, 20 mM, 25 mM, or 30 mM.

The buffers described above can be used for equilibration, loading and/or wash of a mixed-mode chromatography resin during the purification of vedolizumab. In exemplary embodiments, the mixed-mode resin can be a ceramic hydroxyapatite resin.

In some embodiments, two of the equilibration, loading, and wash buffers have the same pH. In some embodiments, two of the equilibration, loading, and wash buffers have the same salt concentration. In some embodiments, two of the equilibration, loading, and wash buffers have the same pH and the same salt concentration. In some embodiments, the equilibration, loading, and wash buffers all have the same pH. In some embodiments, the equilibration, loading, and wash buffers all have the same salt concentration. In some embodiments, the equilibration, loading, and wash buffers all have the same pH and the same salt concentration.

In some embodiments, a method of increasing the yield of vedolizumab recovered following loading a mixed mode chromatography resin, comprises eluting the column at a pH less than 7, pH 5.5 to 6.9 or pH 6.5 to 6.8 and a salt, e.g., NaCl, concentration of 40 to 60 mM or 45 to 55 mM. The method can further comprise washing the column at a pH less than 7, pH 5.5 to 6.9 or pH 6.5 to 6.8.

The buffers and methods described herein can improve the yield of vedolizumab eluted from a mixed-mode chromatography column, relative to the yield when the equilibration, load, and/or wash steps are performed using a buffer having a pH greater than 7.0, and/or a salt concentration greater than 70 mM, and/or a sodium chloride concentration greater than 70 mM. In some embodiments, the yield is improved by at least 1%, 2%, 3%, 4%, 5%, 6%, 7%, 8%, 9%, 10%, 12%, 15%, 18%, 20% or more. In some embodiments the yield is improved by more than 2% relative to a buffer having a pH greater than 7.0. In some embodiments, the yield is improved by more than 3% relative to a buffer having a pH greater than 7.0. In some embodiments, the yield is improved by more than 4% relative to a buffer having a pH greater than 7.0. In some embodiments, the yield is improved by more than 5% relative to a buffer having a pH greater than 7.0.

### VIII. Methods of Assessing Purity of a Composition Containing an Anti-α4β7 Antibody

The purity of a composition comprising an antibody, e.g., vedolizumab, can be assessed by any suitable method, including, but not limited to, the methods described herein.

(a) Assessing Basic Isoform Species

The present invention provides methods of modulating, e.g., reducing, the level of basic isoform species in a composition comprising vedolizumab, or an antigen binding portion thereof, and methods of modulating, e.g., increasing, the level of major isoform in a composition comprising vedolizumab. The relative amount of basic vedolizumab isoform species, and the relative amount of major vedolizumab isoform, present in a vedolizumab composition can be measured using cation exchange chromatography (CEX), as described in detail in the Examples section. The CEX method fractionates antibody species according to overall surface charge. After dilution to low ionic strength using mobile phase, the test sample can be injected onto a CEX column, such as for example a Dionex Pro-Pac™ WCX-10 column (Thermo Fisher Scientific, Waltham, Mass. (USA)), equilibrated in a suitable buffer, e.g., 10 mM sodium phosphate, pH 6.6. The antibody can be eluted using a sodium chloride gradient in the same buffer. Protein elution can be monitored at 280 nm, and peaks are assigned to acidic, basic, or major isoforms categories. Acidic peaks elute from the column with a shorter retention time than the major isoform peak, and basic peaks elute from the column with a longer retention time than the major isoform peak. The percent major isoform, the sum of percent acidic species, and the sum of percent basic species are reported. The major isoform retention time of the sample is compared with that of a reference standard to determine the conformance. In certain embodiments, a CEX-HPLC method is used. For example, a composition comprising vedolizumab and acidic and/or basic species thereof can be resolved using cation exchange chromatography, as described above, followed by analysis of the eluted peaks using HPLC. In one embodiment, HPLC can be performed using an Agilent 1200 HPLC system (Agilent, Santa Clara, Calif.). Quantitation is based on the relative area percent of detected peaks. The CEX-HPLC profile of an exemplary vedolizumab preparation is provided in FIG. 1.

In one embodiment, a CEX assay method comprises diluting a test sample to low ionic strength, injecting onto a CEX column which is equilibrated in 10 mM sodium phosphate, pH 6.6, eluting the column with a NaCl gradient in this buffer, monitoring the peaks at 280 nm and assigning peaks as acidic, main or basic, wherein the acidic peaks elute first with the shortest retention times, the main peak elutes second and the basic peaks elute with the longest retention times, and the peak areas are quantified and their amounts are calculated as the percent of all the peak area.

(b) Assessing Host Cell Protein Levels

The present invention provides methods of modulating, e.g., reducing, the level of residual host cell protein in a composition comprising vedolizumab, or an antigen binding portion thereof. In some embodiments, the amount of host cell protein present in a vedolizumab composition can be measured using enzyme-linked immunosorbent assay (ELISA), using standard techniques. Many ELISA kits designed for this purpose are commercially available, such as the CHO HCP ELISA Kit 3G from Cygnus Technologies (Southport, N.C. (USA)). Host cell proteins in a test sample can be captured using an immobilized polyclonal anti-CHO HCP antibody. Captured proteins can then be detected using a suitable detection agent, for example, a horseradish peroxidase-labeled version of the same antibody. In this exemplary embodiment, the amount of captured peroxidase, which is directly proportional to the concentration of CHO HCP, can be measured colorimetrically at 450 nm using the peroxidase substrate 3,3′,5,5′-tetramethylbenzidine (TMB). The HCP concentration can be determined by comparison to a CHO HCP standard curve, such as that included in the test kit, and is reported as a percentage of the total level of protein in the antibody preparation. In another embodiment, HCP can be determined using the Rabbit-Rabbit (“RaRa”) method exemplified herein. A polyclonal anti-CHO HCP antibody was generated by immunizing rabbits with null cell manufactured harvest material with similar manufacture conditions as vedolizumab, and this antibody was affinity purified. Host CHO cell proteins in vedolizumab samples are captured using immobilized polyclonal anti-CHO HCP antibody, then detected using biotin labeled version of the same antibody and followed by horseradish peroxidase-conjugated streptavidin. The amount of captured peroxidase, which is directly proportional to the concentration of CHO HCP, is measured colorimetrically at 450 nm using the peroxidase substrate 3,3′,5,5′-tetramethylbenzidine (TMB). The HCP concentration is determined by comparison to a CHO HCP standard curve included in the test kit and is reported as a percentage of the total protein. In another embodiment, HCP can be determined using the Rabbit-Goat (“RaGo”) method exemplified herein. Polyclonal anti-CHO HCP antibodies were generated by immunizing rabbits and goats with null cell manufactured harvest material, using similar manufacturing process as that for vedolizumab. The antibody pools were independently affinity purified. Host cell proteins in MLN0002 test samples are captured using immobilized polyclonal rabbit anti-CHO HCP antibody, then detected by sequential addition of the goat anti-CHO affinity purified antibody and a Donkey anti-Goat IgG reagent, labeled with horseradish peroxidase. The amount of captured peroxidase, which is directly proportional to the concentration of CHO HCP, is measured colorimetrically at 450 nm using the peroxidase substrate 3,3′,5,5′-tetramethylbenzidine (TMB). The HCP concentration is determined by comparison to a CHO HCP standard curve included in the test kit and is reported as a (ng/mg) concentration relative to the total vedolizumab.

A CHO HCP assay suitable for use in connection with various embodiments provided herein comprises using a polyclonal anti-CHO HCP antibody to capture HCP, which is detected after binding a horseradish peroxidase-labeled version of the polyclonal anti-CHO HCP antibody which converts the peroxidase substrate 3,3′,5,5′-tetramethylbenzidine (TMB) to a substance that is quantified colorimetrically at 450 nm. In one embodiment, the HCP ELISA suitable for use in connection with various embodiments provided herein is an ELISA method which captures HCP using an immobilized polyclonal anti-CHO HCP antibody, preferably that provided with the CHO HCP ELISA Kit 3G from Cygnus Technologies (Southport, N.C. (USA)), whereby captured proteins are detected by a horseradish peroxidase-labeled version of the same antibody, and the amount of captured peroxidase is measured colorimetrically at 450 nm using 3,3′,5,5′-tetramethylbenzidine (TMB) followed by determination of HCP concentration by comparison to a CHO HCP standard curve.

(c) Assessing Size Variants

In certain embodiments, the levels of aggregates, monomer, and fragments in the chromatographic samples produced using the techniques described herein are analyzed. In various embodiments set forth herein, size exclusion chromatography (SEC) can be used to determine the relative level of monomers, high molecular weight (HMW) aggregates, and low molecular weight (LMW) degradation products present in a population of an antibody or antigen binding portion thereof, e.g., vedolizumab. The SEC method provides size-based separation of antibody monomer from HMW species and LMW degradation products. Test samples and reference standards can be analyzed using commercially available SEC columns, using an appropriate buffer. For example, in some embodiments, SEC analysis can be performed using a G3000 SWxl column (Tosoh Bioscience, King of Prussia, Pa. (USA)), or two G3000 SWxl columns connected in tandem, and an isocratic phosphate-sodium chloride buffer system, pH 6.8. Elution of protein species is monitored at 280 nm. The main peak (monomer) and the total peak area are assessed to determine purity. The purity (%) of the sample (calculated as % monomer), the % HMW aggregate, and/or the % LMW degradation product are reported.

In one embodiment, the SEC analysis comprises injecting a sample onto two G3000 SWxl columns connected in tandem, and run in an isocratic phosphate-sodium chloride buffer system, pH 6.8, wherein the elution of protein species is monitored at 280 nm and the main peak (monomer) and the total peak area are measured.

### IX. Pharmaceutical Compositions and Uses Thereof

Compositions comprising vedolizumab provided herein, e.g., compositions comprising vedolizumab having a reduced level of basic isoform species, and/or compositions comprising vedolizumab having a reduced level of host cell protein, may be incorporated into a pharmaceutical formulation for therapeutic use. Pharmaceutical formulations comprising vedolizumab can be prepared by any suitable method.

In one aspect, a pharmaceutical formulation comprising a composition described herein is a lyophilized pharmaceutical formulation. In one aspect, a lyophilized formulation can be stored as a single dose in one container, e.g., a vial. The container, e.g., vial is stored refrigerated, e.g., at about 2-8° C., or at room temperature, e.g., at about 20° C. to 35° C., about 25° C. or about 30° C., until it is administered to a subject in need thereof. A vial may for example be a 10, 20 or 50 cc vial. The container, e.g., vial may contain about 90 to 115 mg, about 95 to 105 mg, at least about 100 mg, about 135 to 160 mg, about 145 to 155 mg, at least about 150 mg, about 180 to 220 mg, about 190 to 210 mg, about 195 to 205 mg, at least about 200 mg, about 280 mg to 320 mg, about 290 mg to 310 mg, at least about 300 mg, about 380 to 420 mg, about 390 to 410 mg, at least about 400 mg, about 580 to 620 mg, about 590 to 610 mg, or at least about 600 mg of anti-α4β7 antibody. In one aspect, the vial contains about 200 mg of anti-α4β7 antibody. The vial may contain enough of the anti-α4β7 antibody, e.g., vedolizumab, to permit delivery of, e.g., be manufactured to deliver, about 100 mg, about 108 mg, about 150 mg, about 200 mg, about 300 mg, about 400 mg, or about 600 mg of anti-α4β7 antibody. For example, the vial may contain about 15%, about 12%, about 10% or about 8% more anti-α4β7 antibody than the dose amount.

In some embodiments, a composition described herein is formulated as a dry, lyophilized pharmaceutical formulation which can be reconstituted with a liquid, such as sterile water, for administration. Administration of a reconstituted formulation can be by parenteral injection by one of the routes described above. An intravenous injection can be by infusion, such as by further dilution with sterile isotonic saline, buffer, e.g., phosphate-buffered saline or Ringer's (lactated or dextrose) solution.

In some embodiments, a composition described herein is formulated as a liquid pharmaceutical formulation suitable for subcutaneous administration to a human. In some embodiments, the anti-α4β7 antibody is administered by subcutaneous injection, e.g., a dose of about 54 mg, 108 mg or about 165 mg or about 216 mg, at about every two, three or four weeks after the start of therapy or after the third subsequent dose.

In another aspect, a composition described herein comprising an anti-α4β7 antibody, e.g., vedolizumab, is in a stable liquid pharmaceutical composition stored in a container, e.g., a vial, a syringe or cartridge, at about 2-8° C. until it is administered to a subject in need thereof. In some embodiments, the stable liquid pharmaceutical composition of anti-α4β7 antibody comprises about 0% to 5.0%, 0% to 2%, <2%, <1%, <0.6% or <0.5% aggregates. The syringe or cartridge may be a 1 mL or 2 mL container (for example for a 160 mg/mL dose) or more than 2 ml, e.g., for a higher dose (at least 320 mg or 400 mg or higher). The syringe or cartridge may contain at least about 20 mg, at least about 50 mg, at least about 70 mg, at least about 80 mg, at least about 100 mg, at least about 108 mg, at least about 120 mg, at least about 155 mg, at least about 180 mg, at least about 200 mg, at least about 240 mg, at least about 300 mg, at least about 360 mg, at least about 400 mg, or at least about 500 mg of anti-α4β7 antibody. In some embodiments, the container, e.g., syringe or cartridge may be manufactured to deliver about 20 to 120 mg, about 40 mg to 70 mg, about 45 to 65 mg, about 50 to 57 mg or about 54 mg of anti-α4β7 antibody, e.g., vedolizumab. In other embodiments, the syringe or cartridge may be manufactured to deliver about 90 to 120 mg, about 95 to 115 mg, about 100 to 112 mg or about 108 mg of anti-α4β7 antibody, e.g., vedolizumab. In other embodiments, the syringe or cartridge may be manufactured to deliver about 140 to 250 mg, about 150 to 200 mg, about 160 to 170 mg, about 160 to 250 mg, about 175 mg to 210 mg or about 160 mg, about 165 mg, about 180 mg or about 200 mg of anti-α4β7 antibody, e.g., vedolizumab.

Containers that can be used to store and freeze purified compositions described herein include polycarbonate bottles (for IV formulations) or PETG bottles (for subcutaneous formulations). Following aliquoting the formulations to a bottle, freezing may occur (e.g., at −60 degrees Celsius or less).

The pharmaceutical compositions may comprise any vedolizumab composition provided herein, e.g., compositions comprising vedolizumab having a reduced level of basic isoform species, and/or compositions comprising vedolizumab having a reduced level of host cell protein and a pharmaceutically acceptable carrier or excipient. In some embodiments, the pH of the pharmaceutical composition is between 6.0-7.0, for example, pH 6.0-6.2, pH 6.0-6.4, pH 6.0-6.6, pH 6.0-6.8, pH 6.1-6.3, pH 6.1-6.5, pH 6.1-6.7, pH 6.1-6.9, pH 6.2-6.4, pH 6.2-6.6, pH 6.2-6.8, pH 6.2-7.0, pH 6.3-6.5, pH 6.3-6.7, pH 6.3-6.9, pH 6.4-6.6, pH 6.4-6.8, pH 6.4-7.0, pH 6.5-6.7, pH 6.5-6.9, pH p 6.6-pH 6.8, pH 6.6-7.0, pH 6.7-6.9, or pH 6.8-7.0. In some embodiments, the pharmaceutical composition is at a pH of about 6.0, about 6.1, about 6.2, about 6.3, about 6.4, about 6.5, about 6.6, about 6.7, about 6.8, about 6.9, or about 7.0.

The pharmaceutical composition may additionally be supplemented with amino acids or sugars. In some embodiments, the pharmaceutical composition further comprises an amino acid, such as arginine or histidine. In some embodiments, the pharmaceutical composition further comprises a sugar, such as sucrose or trehalose. In some embodiments, the pharmaceutical composition of anti-α4β7 antibody provided herein comprises arginine, histidine, and/or polysorbate 80. In some embodiments, the pharmaceutical composition of anti-α4β7 antibody provided herein comprises citrate, arginine, histidine, and/or polysorbate 80.

Compositions comprising vedolizumab provided herein, e.g., compositions comprising vedolizumab having a reduced level of basic isoform species, and/or compositions comprising vedolizumab having a reduced level of host cell protein, may be used in methods of inhibiting the activity of integrin α4β7 in vitro or in vivo.

In one aspect, a composition described herein can be used for treating a disease or disorder in a subject comprising administering to a subject a composition comprising the anti-α4β7 antibody in an effective amount to treat the disease or disorder in humans. The human subject may be an adult (e.g., 18 years or older), an adolescent, or a child. The human subject may be a person 65 years or older.

In one embodiment, a composition described herein is used to treat a subject who may have had a lack of an adequate response with, loss of response to, or was intolerant to treatment with an immunomodulator, a TNF-alpha antagonist, or combinations thereof. The subject may have previously received treatment with at least one corticosteroid (e.g., prednisone) and had an inadequate response with, were intolerant to, or demonstrated dependence on corticosteroids for treatment, e.g., of inflammatory bowel disease. An inadequate response to corticosteroids refers to signs and symptoms of persistently active disease despite a history of at least one 4-week induction regimen that included a dose equivalent to prednisone 30 mg daily orally for 2 weeks or intravenously for 1 week. A loss of response to corticosteroids refers to two failed attempts to taper corticosteroids to below a dose equivalent to prednisone 10 mg daily orally. Intolerance of corticosteroids includes a history of Cushing's syndrome, osteopenia/osteoporosis, hyperglycemia, insomnia and/or infection.

An immunomodulator may be, for example, oral azathioprine, 6-mercaptopurine, or methotrexate. An inadequate response to an immunomodulator refers to signs and symptoms of persistently active disease despite a history of at least one 8-week regimen or oral azathioprine (greater than or equal to 1.5 mg/kg), 6-mercaptopurine (greater than or equal to 0.75 mg/kg), or methotrexate (greater than or equal to 12.5 mg/week). Intolerance of an immunomodulator includes, but is not limited to, nausea/vomiting, abdominal pain, pancreatitis, LFT abnormalities, lymphopenia, TPMT genetic mutation and/or infection.

A TNFalpha antagonist is, for example, an agent that inhibits the biological activity of TNFalpha, and preferably binds TNFalpha, such as a monoclonal antibody, e.g., REMICADE (infliximab), HUMIRA (adalimumab), CIMZIA (certolizumab pegol), SIMPONI (golimumab) or a circulating receptor fusion protein such as ENBREL (etanercept). An inadequate response to a TNF-alpha antagonist refers to signs and symptoms of persistently active disease despite a history of at least one 4-week induction regimen of infliximab 5 mg/kg IV, 2 doses at least 2 weeks apart; one 80 mg subcutaneous dose of adalimumab, followed by one 40 mg dose at least two weeks apart; or 400 mg subcutaneously of certolizumab pegol, 2 doses at least 2 weeks apart. A loss of response to a TNF-alpha antagonist refers to recurrence of symptoms during maintenance dosing following prior clinical benefit. Intolerance of a TNFalpha antagonist includes, but is not limited to infusion related reaction, demyelination, congestive heart failure, and/or infection.

A loss of maintenance of remission, as used herein for ulcerative colitis subjects, refers to an increase in Mayo score of at least 3 points and a Modified Baron Score of at least 2.

In one embodiment, diseases which can be treated accordingly include, but are not limited to, inflammatory bowel disease (IBD), such as ulcerative colitis, Crohn's disease, ileitis, Celiac disease, nontropical Sprue, enteropathy associated with seronegative arthropathies, microscopic or collagenous colitis, eosinophilic gastroenteritis, or pouchitis resulting after proctocolectomy, and ileoanal anastomosis. In some embodiments, the inflammatory bowel disease is Crohn's disease or ulcerative colitis. Additional diseases which can be treated include, for example, primary sclerosing cholangitis (PSC), and graft-versus-host disease (GVHD).

Ulcerative colitis may be moderate to severely active ulcerative colitis (e.g., having a Mayo score of six to 12 with endoscopy subscore of two or three). Treatment may result in induction and maintenance of clinical response, induction and maintenance of clinical remission, or mucosal healing in patients suffering from moderate to severely active ulcerative colitis. Treatment may also result in a reduction, elimination, or reduction and elimination of corticosteroid use by the patient (e.g., corticosteroid-free remission).

Crohn's disease may be moderate to severely active Crohn's disease (e.g., Crohn's Disease Activity Index (CDAI) score 220 to 450). Treatment may achieve clinical response or achieve clinical remission in patients suffering from moderate to severely active Crohn's disease. Treatment may also result in a reduction, elimination, or reduction and elimination of corticosteroid use by the patient (e.g., corticosteroid-free remission).

Pancreatitis and insulin-dependent diabetes mellitus are other diseases which can be treated using compositions of the invention. It has been reported that MAdCAM (e.g., MAdCAM-1) is expressed by some vessels in the exocrine pancreas from NOD (non-obese diabetic) mice, as well as from BALB/c and SJL mice. Expression of MAdCAM (e.g., MAdCAM-1) was reportedly induced on endothelium in inflamed islets of the pancreas of the NOD mouse, and MAdCAM (e.g., MAdCAM-1) was the predominant addressin expressed by NOD islet endothelium at early stages of insulitis (Hanninen, A., et al., J. Clin. Invest., 92: 2509-2515 (1993)). Treatment of NOD mice with either anti-MAdCAM or anti-beta 7 antibodies prevented the development of diabetes (Yang et al., Diabetes, 46:1542-1547 (1997)). Further, accumulation of lymphocytes expressing α4β7 within islets was observed, and MAdCAM-1 was implicated in the binding of lymphoma cells via α4β7 to vessels from inflamed islets (Hanninen, A., et al., J. Clin. Invest., 92: 2509-2515 (1993)) or to the gastrointestinal tract in mantle cell lymphoma (Geissmann et al., Am. J. Pathol., 153:1701-1705 (1998)).

Examples of inflammatory diseases associated with mucosal tissues which can be treated using a composition of the invention include cholecystitis, cholangitis (Adams and Eksteen Nature Reviews 6:244-251 (2006) Grant et al., Hepatology 33:1065-1072 (2001)), e.g., primary sclerosing cholangitis, Behcet's disease, e.g., of the intestine, or pericholangitis (bile duct and surrounding tissue of the liver), and graft versus host disease (e.g., in the gastrointestinal tract (e.g., after a bone marrow transplant) (Petrovic et al. Blood 103:1542-1547 (2004)). As seen in Crohn's disease, inflammation often extends beyond the mucosal surface, accordingly chronic inflammatory diseases, such as sarcoidosis, chronic gastritis, e.g., autoimmune gastritis (Katakai et al., Int. Immunol., 14:167-175 (2002)) and other idiopathic conditions can be amenable to treatment.

The invention also relates to a method of inhibiting leukocyte infiltration of mucosal tissue. The invention also relates to a method for treating cancer (e.g., an α4β7 positive tumor, such as a lymphoma). Other examples of inflammatory diseases associated with mucosal tissues which can be treated using a formulation of the invention include mastitis (mammary gland) and irritable bowel syndrome.

Diseases or pathogens whose etiologies exploit the interaction of MAdCAM (e.g., MAdCAM-1) with α4β7 can be treated with an anti-α4β7 antibody in a formulation described herein. Examples of such diseases include immunodeficiency disorders, such as caused by human immunodeficiency virus (See, e.g., WO2008140602).

A composition of the invention is administered in an effective amount of the anti-α4β7 antibody which inhibits binding of α4β7 integrin to a ligand thereof. For therapy, an effective amount will be sufficient to achieve the desired therapeutic (including prophylactic) effect (such as an amount sufficient to reduce or prevent α4β7 integrin-mediated binding and/or signaling, thereby inhibiting leukocyte adhesion and infiltration and/or associated cellular responses). An effective amount of an anti-α4β7 antibody, e.g., an effective titer sufficient to maintain saturation, e.g., neutralization, of α4β7 integrin, can induce clinical response or remission in inflammatory bowel disease. An effective amount of an anti-α4β7 antibody can lead to mucosal healing in ulcerative colitis or Crohn's disease. A formulation of the invention can be administered in a unit dose or multiple doses. The dosage can be determined by methods known in the art and can be dependent, for example, upon the individual's age, sensitivity, tolerance and overall well-being. Examples of modes of administration include topical routes such as nasal or inhalational or transdermal administration, enteral routes, such as through a feeding tube or suppository, and parenteral routes, such as intravenous, intramuscular, subcutaneous, intraarterial, intraperitoneal, or intravitreal administration. In one embodiment, the total dose is 165 mg. In another embodiment, the total dose is 108 mg. In another embodiment, the total dose is 216 mg. In another embodiment, the total dose is 300 mg.

In some aspects, the dosing regimen for treating a disease described herein, e.g., UC or Crohn's, has two phases, an induction phase and a maintenance phase. In the induction phase, the antibody or antigen-binding fragment thereof is administered in a way that quickly provides an effective amount of the antibody or antigen binding fragment thereof suitable for certain purposes, such as inducing immune tolerance to the antibody or antigen-binding fragment thereof or for inducing a clinical response and ameliorating inflammatory bowel disease symptoms. A patient can be administered an induction phase treatment when first being treated by an anti-α4β7 antibody, when being treated after a long absence from therapy, e.g., more than three months, more than four months, more than six months, more than nine months, more than one year, more than eighteen months or more than two years since anti-α4β7 antibody therapy or during maintenance phase of anti-α4β7 antibody therapy if there has been a return of inflammatory bowel disease symptoms, e.g., a relapse from remission of disease. In some embodiments, the induction phase regimen results in a higher mean trough serum concentration, e.g., the concentration just before the next dose, than the mean steady state trough serum concentration maintained during the maintenance regimen.

In the maintenance phase, the antibody or antigen-binding fragment thereof is administered in a way that continues the response achieved by induction therapy with a stable level of antibody or antigen-binding fragment thereof. A maintenance regimen can prevent return of symptoms or relapse of inflammatory bowel disease. A maintenance regimen can provide convenience to the patient, e.g., be a simple dosing regimen or require infrequent trips for treatment. In some embodiments, the maintenance regimen can include administration of the anti-α4β7 antibody or antigen-binding fragment thereof, e.g., in a formulation described herein, by a strategy selected from the group consisting of low dose, infrequent administration, self-administration and a combination any of the foregoing.

In one embodiment, e.g., during an induction phase of therapy, the dosing regimen provides an effective amount of an anti-α4β7 antibody or antigen-binding fragment in a formulation described herein for inducing remission of an inflammatory bowel disease in a human patient. The duration of induction phase can be about four weeks, about five weeks, about six weeks, about seven weeks, or about eight weeks of treatment. In some embodiments, the induction regimen can utilize a strategy selected from the group consisting of high dose, frequent administration, and a combination of high dose and frequent administration of the anti-α4β7 antibody or antigen-binding fragment thereof, e.g., in a formulation described herein. Induction dosing can be once, or a plurality of more than one dose, e.g., at least two doses. During induction phase, a dose can be administered once per day, every other day, twice per week, once per week, once every ten days, once every two weeks or once every three weeks. In some embodiments, the induction doses are administered within the first two weeks of therapy with the anti-α4β7 antibody. In one embodiment, induction dosing can be once at initiation of treatment (day 0) and once at about two weeks after initiation of treatment. In another embodiment, the induction phase duration is six weeks. In another embodiment, the induction phase duration is six weeks and a plurality of induction doses are administered during the first two weeks.

In some embodiments, e.g., when initiating treatment of a patient with severe inflammatory bowel disease (e.g., in patients who have failed anti-TNFalpha therapy), the induction phase needs to have a longer duration than for patients with mild or moderate disease. In some embodiments, the induction phase for a patient with a severe disease can have a duration of at least 6 weeks, at least 8 weeks, at least 10 weeks, at least 12 weeks or at least 14 weeks. In one embodiment, an induction dosing regimen for a patient with a severe disease can include a dose at week 0 (initiation of treatment), a dose at week 2 and a dose at week 6. In another embodiment, an induction dosing regimen for a patient with a severe disease can comprise a dose at week 0 (initiation of treatment), a dose at week 2, a dose at week 6 and a dose at week 10.

The dose can be administered once per week, once every 2 weeks, once every 3 weeks, once every 4 weeks, once every 6 weeks, once every 8 weeks or once every 10 weeks. A higher or more frequent dose, e.g., every other day, once per week, once every 2 weeks, once every 3 weeks or once every 4 weeks can be useful for inducing remission of active disease or for treating a new patient, e.g., for inducing tolerance to the anti-α4β7 antibody. A dose once every 2 weeks, once every 3 weeks, once every 4 weeks, once every 5 weeks, once every 6 weeks, once every 8 weeks or once every 10 weeks, can be useful for preventative therapy, e.g., to maintain remission of a patient with chronic disease. In one aspect, the treatment regimen is treatment at day 0, about week 2, about week 6 and every 1 or 2 weeks thereafter. In certain aspects, the treatment regimen is treatment at day 0, about week 2, about week 6 and every 8 weeks thereafter. In another aspect, the induction treatment regimen is treatment every other day for a total of 6 treatments.

In some aspects, a durable clinical remission, for example, a clinical remission which is sustained through at least two, at least three, at least four visits with a caretaking physician within a six month or one year period after beginning treatment, may be achieved with an optimized dosing regimen.

In some aspects, a durable clinical response, for example, a clinical response which is sustained for at least 6 months, at least 9 months, at least a year, after the start of treatment, may be achieved with an effective dosing regimen.

The disclosure is further illustrated by the following examples. The examples provided are for illustrative purposes only, and should not be construed as limiting the scope or content of the disclosure in any way.

### EXAMPLES

The following examples describe various steps in an exemplary process for purification of vedolizumab from a CHO cell culture.

### Example 1. Controlling Charged Isoforms of Vedolizumab

Vedolizumab has three charged isoforms: acidic, major, and basic. Cation exchange (CEX)-HPLC can be used to quantitate the isoform distribution of vedolizumab based on the relative areas of the chromatogram representing the acidic, major, and basic species. An exemplary CEX-HPLC profile depicting these three vedolizumab species is shown in FIG. 1.

Using CEX-HPLC, the charged isoform distribution of vedolizumab was assessed after storage under various conditions. As summarized in Table 1, in-process holds during manufacturing of vedolizumab impacted the distribution of charged isoform species, with the basic isoform being the most impacted by hold conditions.

TABLE 1
Qualitative Changes in Basic Isoform in Process Intermediates

| | | | % Basic Isoform |
|---|---|---|---|
| Process Intermediate | Storage Temp | Storage pH | Change |
| Cell Free Harvest | 2-8° C. | ~ 7.0 | Not determined |
| CEX Load | 2-8° C. | 5.1 | Increases (slowly) |
| CEX Load | Ambient | 5.1 | Increases (fast) |
| CEX Eluate | Ambient | 6.7 | Decreases (slowly) |
| mixed mode Eluate | Ambient | 7.2 | Decreases (fast) |
| AEX | Ambient | 7.2 | Decreases (fast) |

These results indicate that the proportion of different isoform species of vedolizumab can be predictably modulated by holding the antibody under certain conditions. Basic isoform species tend to increase with the duration of the hold at a pH less than approximately 6.5. In contrast, basic isoform species decrease with the duration of the hold at a pH greater than approximately 6.5. These changes are observed with vedolizumab produced both at pilot scale (Table 2) and manufacturing scale (Table 3).

TABLE 2
Vedolizumab isoforms during in-process holds - pilot purification

| | | | | CEX-HPLC | | |
|---|---|---|---|---|---|---|
| | | | Time- | | % | |
| | Hold | Storage | point | % | Major | % |
| In-Process Step | Temp. | pH | (Days) | Acidic | Isoform | Basic |
| CEX Load | 2-8° C. | 5.1 | 0 | 22.5 | 67.8 | 9.8 |
| | | 5.1 | 1 | 22.4 | 66.8 | 10.8 |
| | | 5.1 | 3 | 22.3 | 66.2 | 11.4 |
| | Ambient | 5.1 | 4 | 21.9 | 65.9 | 12.3 |
| CEX Eluate | Ambient | 6.7 | 0 | 22.3 | 66.4 | 11.2 |
| | | 6.7 | 1 | 22.5 | 66.4 | 11.1 |
| | | 6.7 | 4 | 23.0 | 66.4 | 10.6 |
| Mixed mode Eluate | Ambient | 7.2 | 0 | 22.5 | 68.0 | 9.5 |
| | | 7.2 | 1 | 22.6 | 68.6 | 8.8 |
| | | 7.2 | 3 | 23.1 | 68.9 | 8.0 |
| AEX Flow Through | Ambient | 7.2 | 0 | 23.1 | 68.9 | 7.9 |
| Material | | 7.2 | 1 | 23.3 | 69.0 | 7.7 |
| | | 7.2 | 4 | 23.7 | 69.7 | 6.6 |

TABLE 3
Vedolizumab isoforms during in-process holds - manufacturing-scale purification

| In-Process | Storage | CEX | Timepoints - Days | | | | | | | |
|---|---|---|---|---|---|---|---|---|---|---|
| Step | Conditions | Results | 0 | 1 | 2 | 3 | 4 | 5 | 6 | 7 |
| CEX Load | Stainless | % Acidic | 20.5 | 20.4 | 20.2 | 20.3 | 20.2 | 20.2 | | |
| | Steel, | % Major | 67.0 | 66.8 | 66.8 | 66.5 | 66.6 | 66.4 | | |
| | 2-8° C., | % Basic | 12.6 | 12.8 | 12.9 | 13.2 | 13.2 | 13.4 | | |
| | pH 5.1 | | | | | | | | | |
| CEX Load | Stainless | % Acidic | 20.5 | 20.2 | 20.0 | 20.1 | 20.1 | 20.0 | | |
| | Steel, | % Major | 67.0 | 66.3 | 66.3 | 66.0 | 65.8 | 65.7 | | |
| | Ambient | % Basic | 12.6 | 13.5 | 13.7 | 13.9 | 14.0 | 14.2 | | |
| | until T1, | | | | | | | | | |
| | then at 2- | | | | | | | | | |
| | 8° C., | | | | | | | | | |
| | pH 5.1 | | | | | | | | | |
| Mixed | Stainless | % Acidic | 20.4 | 20.7 | 20.9 | 21.1 | 21.2 | 21.2 | | 21.4 |
| mode | Steel, | % Major | 68.0 | 68.1 | 68.3 | 68.5 | 68.8 | 68.9 | | 69.1 |
| Eluate | Ambient, | % Basic | 11.6 | 11.2 | 10.8 | 10.4 | 10.0 | 9.8 | | 9.5 |
| | pH 7.2 | | | | | | | | | |
| AEX Flow | Stainless | % Acidic | 20.7 | 21.0 | 21.1 | 21.3 | 21.4 | | 21.7 | |
| Through | Steel, | % Major | 68.3 | 68.4 | 68.6 | 69.0 | 69.1 | | 69.0 | |
| Material | Ambient, | % Basic | 11.0 | 10.5 | 10.3 | 9.7 | 9.5 | | 9.3 | |
| | pH 7.2 | | | | | | | | | |

An additional experiment was performed to assess the charged isoform distribution of vedolizumab derived from GS-CHO cells. The percentage of major, acidic, and basic species of vedolizumab was assessed after storage for 0-7 days at 5° C. or room temperature at a variety of pHs (i.e., pH 4.7, 5.1, 5.3, 5.7, 5.9, 6.1, 6.5, or 6.9). As shown in Tables 4-11, improved stability was observed at pHs greater than or equal to pH 5.9. Following Protein A capture and elution, typical pH ranges for neutralization (pH˜4.9 to 5.2) were associated with increased formation of basic species. The increase in basic species was slowed when vedolizumab was stored at pH 5.9 or 6.1 relative to basic species observed during storage at pH<5.9. As shown in Tables 10 and 11, the increase in basic species at pH greater than or equal to pH 6.5 is halted or even reversed.

TABLE 4
Vedolizumab isoform distribution s
during in-proces hold at pH 4.7

| Storage | Hold | Acidic | Major | Basic |
|---|---|---|---|---|
| Temperature (° C.) | Day | % | % | % |
| T0 | 0 | 14.40 | 71.56 | 14.04 |
| 5° C. | 1 | 14.22 | 71.72 | 14.07 |
| 5° C. | 2 | 14.26 | 71.33 | 14.41 |
| 5° C. | 3 | 14.34 | 70.92 | 14.74 |
| 5° C. | 5 | 13.99 | 70.98 | 15.03 |
| 5° C. | 7 | 14.03 | 70.72 | 15.26 |
| Room Temperature | 1 | 13.99 | 70.95 | 15.06 |
| Room Temperature | 2 | 13.68 | 68.90 | 17.42 |
| Room Temperature | 3 | 13.05 | 67.90 | 19.05 |
| Room Temperature | 5 | 12.88 | 65.06 | 22.06 |
| Room Temperature | 7 | 12.29 | 62.18 | 25.54 |

TABLE 5
Vedolizumab isoform distribution
during in-process hold at pH 5.1

| Storage | Hold | Acidic | Major | Basic |
|---|---|---|---|---|
| Temperature (° C.) | Day | % | % | % |
| T0 | 0 | 14.37 | 71.60 | 14.02 |
| 5° C. | 1 | 14.21 | 71.38 | 14.42 |
| 5° C. | 2 | 14.09 | 71.39 | 14.52 |
| 5° C. | 3 | 14.25 | 71.47 | 14.28 |
| 5° C. | 5 | 14.07 | 71.19 | 14.74 |
| 5° C. | 7 | 14.15 | 70.51 | 15.34 |
| Room Temperature | 1 | 13.95 | 71.20 | 14.85 |
| Room Temperature | 2 | 13.65 | 69.63 | 16.72 |
| Room Temperature | 3 | 13.55 | 67.97 | 18.48 |
| Room Temperature | 5 | 13.07 | 66.01 | 20.92 |
| Room Temperature | 7 | 12.63 | 63.18 | 24.19 |

TABLE 6
Vedolizumab isoform distribution
during in-process hold at pH 5.3

| Storage | Hold | Acidic | Major | Basic |
|---|---|---|---|---|
| Temperature(° C.) | Day | % | % | % |
| T0 | 0 | 14.23 | 72.22 | 13.55 |
| 5° C. | 1 | 14.36 | 71.61 | 14.03 |
| 5° C. | 2 | 14.36 | 71.02 | 14.62 |
| 5° C. | 3 | 14.21 | 71.59 | 14.20 |
| 5° C. | 5 | 13.98 | 71.06 | 14.96 |
| 5° C. | 7 | 14.00 | 70.94 | 15.06 |
| Room Temperature | 1 | 14.03 | 71.04 | 14.94 |
| Room Temperature | 2 | 13.83 | 70.03 | 16.15 |
| Room Temperature | 3 | 13.48 | 68.68 | 17.84 |
| Room Temperature | 5 | 13.29 | 66.88 | 19.84 |
| Room Temperature | 7 | 12.77 | 64.93 | 22.30 |

TABLE 7
Vedolizumab isoform distribution
during in-process hold at pH 5.7

| Storage | Hold | Acidic | Major | Basic |
|---|---|---|---|---|
| Temperature (° C.) | Day | % | % | % |
| T0 | 0 | 14.34 | 71.49 | 14.17 |
| 5° C. | 1 | 14.56 | 71.66 | 13.78 |
| 5° C. | 2 | 14.49 | 71.87 | 13.64 |
| 5° C. | 3 | 14.81 | 71.62 | 13.58 |
| 5° C. | 5 | 14.37 | 71.15 | 14.47 |
| 5° C. | 7 | 14.30 | 70.94 | 14.76 |
| Room Temperature | 1 | 14.59 | 71.09 | 14.32 |
| Room Temperature | 2 | 14.09 | 70.42 | 15.49 |
| Room Temperature | 3 | 14.14 | 69.11 | 16.75 |
| Room Temperature | 5 | 13.80 | 68.27 | 17.94 |
| Room Temperature | 7 | 13.82 | 66.46 | 19.73 |

TABLE 8
Vedolizumab isoform distribution
during in-process hold at pH 5.9

| Storage | Hold | Acidic | Major | Basic |
|---|---|---|---|---|
| Temperature (° C.) | Day | % | % | % |
| T0 | 0 | 14.62 | 71.53 | 13.86 |
| 5° C. | 1 | 14.29 | 71.59 | 14.13 |
| 5° C. | 2 | 14.57 | 71.57 | 13.86 |
| 5° C. | 3 | 14.44 | 71.29 | 14.27 |
| 5° C. | 5 | 14.32 | 71.34 | 14.34 |
| 5° C. | 7 | 14.50 | 71.29 | 14.21 |
| Room Temperature | 1 | 14.34 | 71.18 | 14.48 |
| Room Temperature | 2 | 14.31 | 70.88 | 14.81 |
| Room Temperature | 3 | 14.26 | 70.46 | 15.28 |
| Room Temperature | 5 | 14.47 | 68.98 | 16.55 |
| Room Temperature | 7 | 14.09 | 68.08 | 17.84 |

TABLE 9
Vedolizumab isoform distribution
during in-process hold at pH 6.1

| Storage | Hold | Acidic | Major | Basic |
|---|---|---|---|---|
| Temperature (° C.) | Day | % | % | % |
| T0 | 0 | 14.52 | 71.73 | 13.75 |
| 5° C. | 1 | 14.54 | 71.51 | 13.95 |
| 5° C. | 2 | 14.39 | 71.57 | 14.04 |
| 5° C. | 3 | 14.42 | 71.69 | 13.89 |
| 5° C. | 5 | 14.38 | 71.80 | 13.82 |
| 5° C. | 7 | 14.58 | 71.21 | 14.21 |
| Room Temperature | 1 | 14.35 | 71.61 | 14.04 |
| Room Temperature | 2 | 14.33 | 71.18 | 14.49 |
| Room Temperature | 3 | 14.55 | 70.64 | 14.81 |
| Room Temperature | 5 | 14.38 | 69.88 | 15.74 |
| Room Temperature | 7 | 14.27 | 69.40 | 16.33 |

Vedolizumab isoform distribution
during in-process hold at pH 6.5

| Storage | Hold | Acidic | Major | Basic |
|---|---|---|---|---|
| Temperature (° C.) | Day | % | % | % |
| T0 | 0 | 14.49 | 71.70 | 13.81 |
| 5° C. | 3 | 14.30 | 72.30 | 13.40 |
| 5° C. | 7 | 14.33 | 72.03 | 13.65 |
| Room Temperature | 1 | 14.52 | 71.57 | 13.91 |
| Room Temperature | 2 | 14.64 | 71.47 | 13.89 |
| Room Temperature | 3 | 14.68 | 71.72 | 13.60 |
| Room Temperature | 5 | 14.71 | 71.60 | 13.69 |
| Room Temperature | 7 | 14.86 | 71.00 | 14.15 |

TABLE 11
Vedolizumab isoform distribution
during in-process hold at pH 6.9

| Storage | Hold | Acidic | Major | Basic |
|---|---|---|---|---|
| Temperature(° C.) | Day | % | % | % |
| T0 | 0 | 14.42 | 71.34 | 14.25 |
| 5C | 3 | 14.59 | 71.58 | 13.82 |
| 5C | 7 | 14.71 | 71.60 | 13.69 |
| Room Temperature | 1 | 14.96 | 71.56 | 13.48 |
| Room Temperature | 2 | 15.08 | 71.87 | 13.05 |
| Room Temperature | 3 | 15.09 | 72.14 | 12.77 |
| Room Temperature | 5 | 15.58 | 72.39 | 12.03 |
| Room Temperature | 7 | 15.96 | 71.88 | 12.16 |

This data indicates that the level of the major isoform and the level of basic isoform species in a vedolizumab preparation can be modulated by pH. In particular, basic isoform species are increased by exposure of the antibody to low pH (<pH 5.9), with an accompanying decrease in major isoform. The level of basic isoform species increases more rapidly, and to a greater extent, with exposure to decreasing pH. In addition, this trend is reversible by exposure to elevated pH, e.g., >pH 6.5.

### Example 2. Reducing Basic Isoforms of Vedolizumab

To further assess the impact of pH on the formation of basic isoforms of vedolizumab, the antibody was exposed to a high pH condition (200 mM Tris, pH 9), and cation exchange (CEX)-HPLC was used to quantitate the isoform distribution of vedolizumab. For each condition, the relative amount of each vedolizumab isoform was quantified by determining the relative area under the chromatogram peak corresponding to the acidic, main, and basic isoforms.

As shown in Table 12, three peaks corresponding to basic species of vedolizumab were present at pH 6.3 (control) (i.e., “Basic Peak 1,” “Basic Peak 2,” and “Basic Peak 3”). At elevated pH, a significant decrease in Basic Peak 2 was observed, as shown in Table 12.

TABLE 12
Sensitivity of Basic Peak 2 to Elevated pH

| | | Following Exposure to |
|---|---|---|
| | Control | 200 mM Tris HC1, pH 9 |
| Name | % Area | % Area |
| Basic Peak-1 | 6.85 | 6.8 |
| Basic Peak-2 | 3.09 | 0.85 |
| Basic Peak-3 | 0.83 | 0.56 |

Material eluting from a CEX resin with a retention time characteristic of Basic Peak 2 was collected and subjected to enzymatic digestion, followed by analysis using mass spectrometry (MS). A MS peak characteristic of succinimide was present in the Basic Peak 2 CEX fraction from the control preparation, but absent from the preparation analyzed following exposure to pH 9. Analysis of the primary amino acid sequence of vedolizumab identified an aspartic acid residue in CDR-H3 of vedolizumab positioned near residues that favor the isomerization of aspartic acid to succinimide, i.e., glycine or serine at the n+1 position (CDR-H3: GGYDGWDYAIDY (SEQ ID NO: 4)). This finding suggests that the increase in basic species of vedolizumab observed at low pH may be attributable to isomerization of this aspartic acid residue to succinimide. Maintenance of the antibody at or near neutral pH can slow or prevent the formation of Basic Peak 2, and treatment with elevated pH (>pH 6.9) can reverse the isomerization reaction, converting succinimide back to aspartic acid (or isoaspartic acid).

To further assess the impact of pH on Basic Peak 2, the relative peak area was determined by CEX-HPLC following exposure of the antibody to varying pH conditions (pH 6.5, pH 7, pH 8, pH 8.5, or pH 9). As shown in Table 13, Basic Peak 2 was highly sensitive to pH, and decreased with increasing pH, consistent with the findings reported in Example 1.

TABLE 13
Loss of Basic Peak 2 at Elevated pH

| pH | % Peak Area | % Loss |
|---|---|---|
| 6.5 | 3.3 | 0.0 |
| 7 | 3.26 | 1.2 |
| 8 | 2.13 | 35.5 |
| 8.5 | 1.36 | 58.8 |
| 9 | 0.69 | 79.1 |

The level of the vedolizumab isoform corresponding to Basic Peak 2 was then assessed based on vedolizumab preparations derived from two distinct CHO cell lines (DHFR-CHO and GS-CHO). As shown in Table 14, reduction of Basic Peak 2 was observed in vedolizumab preparations derived from both DHFR-CHO and GS-CHO cell lines.

TABLE 14
Loss of Basic Peak 2 in Antibody Preparation
Derived from Two Distinct CHO Cell Lines

| | DHFR-CHO | | GS-CHO | |
|---|---|---|---|---|
| Peak | Control | Tris, pH 9 | Control | Tris, pH 9 |
| Basic-1 | 4.9 | 4.7 | 6.9 | 7.0 |
| Basic-2 | 2.0 | 0.6 | 3.4 | 0.7 |
| Basic-3 | 1.2 | 1.1 | 1.2 | 0.9 |

### Example 3. Influence of Anion Exchange Load Conductivity on Host Cell Protein Clearance

As it is generally desirable to reduce the amount of host cell protein contaminants in therapeutic protein compositions, manufacturing methods for producing a vedolizumab composition with reduced host cell protein content were examined.

The standard operating range of buffer conductivity for anion exchange (AEX) (e.g., via an anion exchange Q membrane adsorber), is approximately 11-15 mS/cm (average approximately 13.6 mS/cm). To assess impurity clearance at conditions beyond standard operating range, impurity clearance was tested in compositions obtained using lower conductivity AEX conditions. Two independent starting preparations of vedolizumab clarified harvest were tested (Harvest 1 and Harvest 2). All samples were subjected to AEX purification after adjusting the conductivity of the load material to standard conductivity (˜13.6 mS/cm), or low conductivity (˜11 mS/cm). As shown in Table 15, HCP levels decreased with a low conductivity AEX load material as compared to a standard conductivity AEX load material.

TABLE 15
HCP clearance using a rabbit-goat (RaGo) process specific ELISA

| | Clarified Harvest 1 | Clarified Harvest 2 |
|---|---|---|
| Process Step | HCP ppm | HCP ppm |
| Clarified Harvest | 448,000 | 380,000 |
| Mixed Mode Eluate | 22.5 | 7.88 |
| Following AEX (Standard | 11.1 | 3.79 |
| Conductivity Load) | | |
| Following AEX (Low | 4.82 | <3.22 |
| Conductivity Load) | | |

Next, AEX performance (in flow-through mode) was tested with loading buffers having a range of conductivities below the standard operating conditions (i.e., below 13-15 mS/cm). Table 16 summarizes the loading condition, conductivity, host cell protein content, and percentage of acidic and basic isoforms. Testing was performed using CHO host cell protein ELISA. As shown in Table 16, the amount of host cell protein decreased with decreasing AEX loading buffer conductivity.

TABLE 16
Conductivity ranging study - Q Membrane Performance

| | Amount | | | | | HPLC_CEX | | |
|---|---|---|---|---|---|---|---|---|
| | of Water | % | | | | | % | |
| Load | Added | Water | Cond. @ | Cond. @ | HCP | % | Main | % |
| Condition | (mL) | Added | 21° C. | 25° C. | (ppm) | Acidic | Isoform | Basic |
| Mixed mode | NA | NA | 13.35 | | 39.3 | 24.1 | 65.5 | 10.4 |
| Eluate Pool | | | | | | | | |
| 11 mS/cm | 24 | 24.2% | 10.96 | 11.00 | 6.87 | 24.5 | 65.8 | 9.7 |
| Load | | | | | | | | |
| 10.5 mS/cm | 32 | 32.3% | 10.45 | 10.48 | 6.74 | 24.4 | 65.6 | 10.0 |
| Load | | | | | | | | |
| 10 mS/cm | 38 | 38.4% | 9.986 | 10.07 | 5.15 | 24.5 | 65.5 | 10.1 |
| Load | | | | | | | | |
| 9.5 mS/cm | 46 | 46.5% | 9.425 | 9.44 | 5.03 | 24.4 | 65.7 | 9.8 |
| Load | | | | | | | | |
| 9 mS/cm | 54 | 54.5% | 8.965 | 8.97 | <14.00 | 24.6 | 65.7 | 9.7 |
| Load | | | | | | | | |

The impact of lowered AEX conductivity on HCP clearance was then assessed by two different HCP ELISA methods. In the first method, a rabbit primary antibody and an anti-rabbit secondary antibody were used in a HCP ELISA (RaRa). The second method utilized a rabbit primary antibody and a goat secondary antibody in the HCP ELISA (RaGo). Tables 17 and 18 summarize the results of these two HCP ELISA methods following use of a standard or low conductivity AEX load, respectively. Both methods demonstrate that HCP clearance is improved by reducing the conductivity of the AEX load material.

TABLE 17
Standard conductivity AEX load conditions (~13.3 mS/cm)
Vedolizumab Purified with Standard Conductivity AEX Load

| | | RaRa | RaGo |
|---|---|---|---|
| Sample ID | Process Step | ppm | ppm |
| A | Clarified Harvest | 784,961 | 484,000 |
| B | Mixed Mode Eluate | 7.67 | 11.3 |
| C | AEX (std) | 4.77 | 5.81 |

TABLE 18
Low conductivity AEX load conditions (~10.0 mS/cm)
Vedolizumab Purified with Low Conductivity AEX Load

| | | RaRa | RaGo |
|---|---|---|---|
| Sample ID | Process Step | ppm | ppm |
| A | Clarified Harvest | 784,961 | 484,000 |
| B | Mixed Mode Eluate | 7.67 | 11.3 |
| C | AEX (std) | 3.07 | <2.44 |

The amount of HCP in recombinant protein preparations is variable. As shown herein, HCP clearance from preparations of vedolizumab can be improved by reducing the conductivity during purification using AEX. Irrespective of the HCP concentration in the starting material, lower conductivity AEX conditions achieved a greater reduction in HCP as compared to standard, higher conductivity AEX conditions.

### Example 4. Impact of Equilibration and Wash Conditions on the Loading Capacity of a Mixed Mode Resin

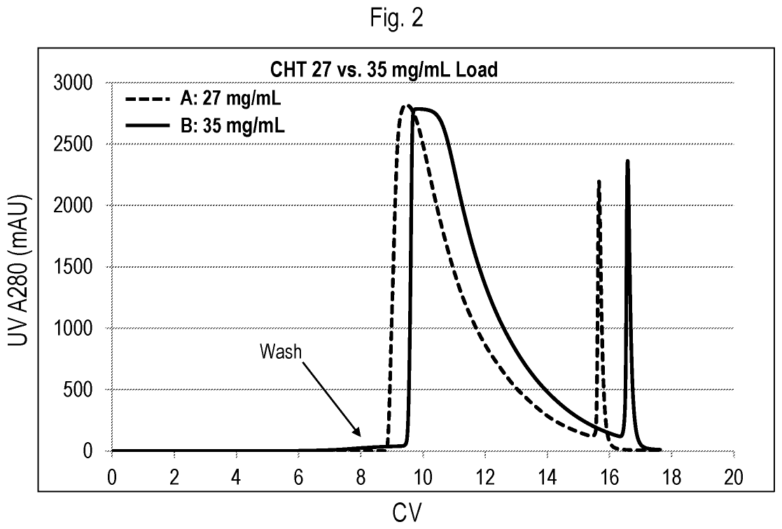

In order to minimize the loss of vedolizumab during purification, various purification conditions were examined to identify steps at which the yield of the antibody product is reduced. It was observed that protein loss occurs during the wash step of ceramic hydroxyapatite (CHT) purification under high concentration loading conditions. For example, FIG. 2 compares the elution profile of vedolizumab from a CHT column following loading of 27 mg/ml or 35 mg/ml using a standard wash and equilibration buffer (75 mM NaCl, 10 mM Sodium Phosphate, pH 7.2). As shown in FIG. 2, protein began to bleed off of the column during the wash step under high concentration loading conditions (35 mg/ml).

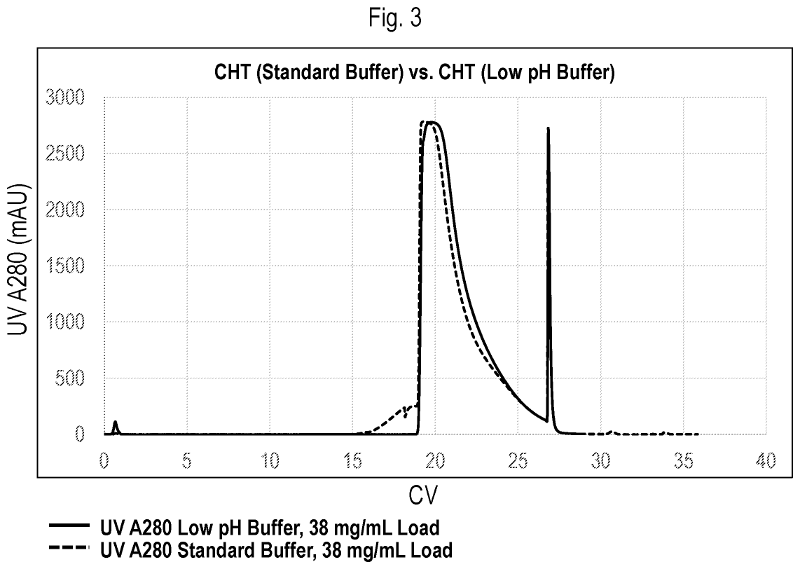

To increase the loading capacity of vedolizumab on the CHT column, and to improve the yield of vedolizumab by reducing the amount of protein lost in the CHT wash step, an alternative CHT equilibration, load, and wash buffer was assessed. The vedolizumab load amount (g/l) on the CHT column and resulting yield achieved was determined after operation of the CHT column using a lower pH buffer (50 mM NaCl, 10 mM sodium phosphate, pH 6.7) for CHT equilibration and wash, as compared to operation of the CHT column using a standard CHT buffer at an elevated pH (75 mM NaCl, 10 mM sodium phosphate, pH 7.2). As shown in FIG. 3, there was a significant amount of protein lost during the wash step using standard CHT buffer conditions (dashed line), but there was no protein loss observed using the low pH buffer (solid line). Further, as summarized in Table 19, use of the low pH buffer at the equilibration and wash steps allowed for a greater loading volume on the CHT column, and resulted in 2-3% higher yield without altering product quality (e.g., percent aggregates) in the final eluate, as compared to that achieved using the standard CHT buffer.

TABLE 19
Analytical Comparison: Standard CHT vs. CHT with low pH Buffers

| | | | Total | | | | HPLC - |
|---|---|---|---|---|---|---|---|
| | | | Protein | | HPLC - | HPLC - | CEX |
| | | Eluate | (mg/ml | | SEC | SEC | % |
| | Loading | Volume | by | Yield | % | % | Major |
| Sample | (mg/mL) | (CV) | A280) | % | Aggregates | Monomer | Isoform |
| CHT Load | | NA | 12.5 | NA | 1.2 | 98.3 | 63.0 |
| Buffer: | 38 | 6.8 | 5.00 | 87.9% | 0.2 | 99.3 | 63.7 |
| Standard | | | | | | | |
| Buffer: | 38 | 7.0 | 4.80 | 89.0% | 0.3 | 99.2 | 64.0 |
| Standard | | | | | | | |
| Buffer: | 38 | 6.8 | 5.00 | 88.3% | 0.2 | 99.2 | 63.8 |
| Standard | | | | | | | |
| Buffer: | 35 | 6.1 | 5.0 | 89.5% | 0.2 | 99.4 | 66.6 |
| Standard | | | | | | | |
| Buffer: | 27 | 5.8 | 4.1 | 89.9% | 0.1 | 99.4 | 66.5 |
| Standard | | | | | | | |
| Buffer: low | 38 | 7.0 | 5.14 | 93.3% | 0.2 | 99.3 | 64.0 |
| pH Buffer | | | | | | | |
| Buffer: low | 45 | 7.2 | 5.86 | 92.8% | 0.3 | 99.2 | 63.6 |
| pH Buffer | | | | | | | |

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

SEQUENCE LISTING TABLE

| SEQ | | |
|---|---|---|
| ID | | |
| NO: | DESCRIPTION | SEQUENCE |
| 1 | Heavy chain | QVQLVQSGAEVKKPGASVKVSCKGSGYTFTSYWMHWVRQA |
| | (HC) variable | PGQRLEWIGEIDPSESNTNYNQKFKGRVTLTVDISASTAYMEL |
| | region (amino | SSLRSEDTAVYYCARGGYDGWDYAIDYWGQGTLVTVSS |
| | acid) | |
| 2 | HC CDR1 | SYWMH |
| | (amino acid) | |
| 3 | HC CDR2 | EIDPSESNTNYNQKFKG |
| | (amino acid) | |
| 4 | HC CDR3 | GGYDGWDYAIDY |
| | (amino acid) | |
| 5 | Light chain | DVVMTQSPLSLPVTPGEPASISCRSSQSLAKSYGNTYLSWYLQ |
| | (LC) variable | KPGQSPQLLIYGISNRFSGVPDRFSGSGSGTDFTLKISRVEAED |
| | region (amino | VGVYYCLQGTHQPYTFGQGTKVEIK |
| | acid) | |
| 6 | LC CDR1 | RSSQSLAKSYGNTYLS |
| | (amino acid) | |
| 7 | LC CDR2 | GISNRFS |
| | (amino acid) | |
| 8 | LC CDR3 | LQGTHQPYT |
| | (amino acid) | |
| 9 | Heavy chain | QVQLVQSGAEVKKPGASVKVSCKGSGYTFTSYWMHWVRQA |
| | amino acid | PGQRLEWIGEIDPSESNTNYNQKFKGRVTLTVDISASTAYMEL |
| | sequence | SSLRSEDTAVYYCARGGYDGWDYAIDYWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKPS |
| | | NTKVDKKVEPKSCDKTHTCPPCPAPELAGAPSVFLFPPKPKDT |
| | | LMISRTPEVTCVVVDVSHEDPEVKFNWYVDGVEVHNAKTKP |
| | | REEQYNSTYRVVSVLTVLHQDWLNGKEYKCKVSNKALPAPI |
| | | EKTISKAKGQPREPQVYTLPPSRDELTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 10 | Light chain | DVVMTQSPLSLPVTPGEPASISCRSSQSLAKSYGNTYLSWYLQ |
| | amino acid | KPGQSPQLLIYGISNRFSGVPDRFSGSGSGTDFTLKISRVEAED |
| | sequence | VGVYYCLQGTHQPYTFGQGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |

1. A method of producing a composition comprising vedolizumab, comprising:
providing a composition comprising vedolizumab at a pH greater than pH 6.5; and
incubating the composition comprising vedolizumab for a period of at least 20 minutes-10 hours; wherein the method reduces the level of basic vedolizumab isoform species,
thereby producing a composition comprising vedolizumab having a reduced level of basic isoform species.

2. (canceled)

3. The method of claim 1, wherein the method produces a composition comprising vedolizumab having <16%, <15%, <14%, <13%, <12%, <11% or <10% basic vedolizumab isoform species.

4. The method of claim 1, wherein the incubation is performed during vedolizumab purification, and wherein the incubation is performed (a) prior to ultrafiltration/diafiltration (UF/DF) of the antibody, or (b) prior to formulation of the antibody in a pharmaceutically acceptable buffer.

5. The method of claim 1, wherein the incubation is performed at ambient temperature, wherein the incubation is performed at 15-30° C., or wherein the incubation is performed at 20-25° C.

6. (canceled)

7. The method of claim 1, wherein:
the composition comprising vedolizumab is provided at a pH of about 6.5-8.5, at a pH of about 7.0-8.0, or at a pH of about 7.0-7.5; or
the composition comprising vedolizumab is provided at a pH of about pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, or pH 8.5.

8-10. (canceled)

11. The method of claim 1, wherein:
the composition comprising vedolizumab is incubated for a period of about 10-120 hours a period of about 12-120 hours, a period of about 12-96 hours, a period of about 12-72 hours, a period of about 12-48 hours, a period of about 24-120 hours, a period of about 24-96 hours, a period of about 24-72 hours, or a period of about 24-48 hours;
the composition comprising vedolizumab is incubated for a period of at least 12 hours; or
the composition comprising vedolizumab is incubated for a period of about 12 hours, about 24 hours, about 36 hours, about 48 hours, about 72 hours, about 96 hours, or about 120 hours.

12-21. (canceled)

22. A method of purifying a humanized anti-α4β7 antibody or an antigen binding portion thereof from a clarified cell culture harvest comprising
(i) providing a clarified cell culture harvest obtained from a culture of recombinant host cells expressing the anti-α4β7 antibody or an antigen binding portion thereof, and
(ii) purifying the anti-α4β7 antibody or an antigen binding portion thereof from the cell culture harvest, wherein the antibody is exposed to a pH at or below 4.0 for no more than 24 hours,
wherein the anti-α4β7 antibody or antigen binding portion thereof comprises a heavy chain variable region comprising the amino acid sequence set forth in SEQ ID NO:1, and a light chain variable region comprising the amino acid sequence set forth in SEQ ID NO:5.

23. The method of claim 22, wherein the anti-α4β7 antibody, or an antigen binding portion thereof, has a reduced level of basic isoform species (determined by CEX) as compared to a control, wherein the control is a composition comprising the anti-α4β7 antibody, or an antigen binding portion thereof, produced by the same method, wherein the antibody is exposed to a pH at or below 4.0 (e.g., pH 3.6-4.0) for a longer duration of time, i.e., greater than 24 hours.

24. The method of claim 22, wherein the anti-α4β7 antibody, or an antigen binding portion thereof is vedolizumab, or an antigen binding portion thereof.

25. The method of claim 24, wherein the composition comprising vedolizumab, or an antigen binding portion thereof, comprises a first basic isoform peak (BP1) and a second basic isoform peak (BP2), and wherein the method produces a composition comprising vedolizumab, or an antigen binding portion thereof, having a reduced level of BP2.

26. The method of claim 25, wherein the method produces a composition comprising vedolizumab, or an antigen binding portion thereof, having less than 2%, less than 1.5%, less than 1%, or less than 0.7% BP2.

27. The method of claim 24, wherein the composition comprising vedolizumab is derived from a mammalian cell culture expressing vedolizumab.

28-30. (canceled)

31. The method of claim 27, wherein the method further comprises purifying the composition comprising vedolizumab from mammalian host cell protein (HCP) using one or more chromatographic separation steps selected from the group consisting of affinity chromatography, cation exchange chromatography, anion exchange chromatography, and ceramic hydroxyapatite (CHT) chromatography.

32-43. (canceled)

44. The method of claim 22, wherein the method comprises incorporating the composition into a pharmaceutical formulation.

45. The method of claim 44, wherein the pharmaceutical formulation is a lyophilized pharmaceutical formulation or a liquid pharmaceutical formulation.

46-48. (canceled)

49. A composition comprising vedolizumab, wherein the composition is produced by or is obtainable by the method of claim 1.

50. (canceled)

51. The composition of claim 49, wherein the basic vedolizumab isoform species comprises less than 16%, less than 15%, less than 14%, less than 13%, less than 12%, less than 11%, or less than 10% of the vedolizumab species present in the composition.

52. A low basic species composition comprising an anti-α4β7 antibody, wherein the composition comprises less than 16%, less than 15%, less than 14%, less than 13%, less than 12%, less than 11%, or less than 10% total basic isoform species of the anti-α4β7 antibody, wherein the basic isoform species have a net positive charge relative to a main isoform of the anti-α4β7 antibody and can be quantified by determining the relative area of peaks that elute more slowly from a cation exchange (CEX) resin than a peak corresponding to the main isoform, and wherein the anti-α4β7 antibody comprises a heavy chain variable region comprising SEQ ID NO:1, and a light chain variable region comprising SEQ ID NO:5.

53. The composition of claim 52, wherein the composition comprises a first basic isoform peak (BP1) and a second basic isoform peak (BP2).

54. The composition of claim 53, wherein the composition comprises less than 2% BP2, less than 1.5% BP2, less than 1% BP2, or less than 0.7% BP2.

55-57. (canceled)

58. The composition of claim 53, wherein the ratio of BP1 to BP2 is at least 3, at least 5, at least 7, or at least 10.

59-61. (canceled)

62. A pharmaceutical composition comprising the composition of claim 52 and a pharmaceutically acceptable carrier or excipient.

63. A method of producing a composition comprising vedolizumab, comprising:
(a) contacting a sample containing vedolizumab and host cell protein (HCP) with an anion exchange resin in the presence of a loading buffer, wherein the loading buffer has a conductivity of 11 mS/cm or less, such that HCP binds to the anion exchange resin; and
(b) collecting the flow through material from the anion exchange resin,
wherein the flow through material comprises vedolizumab and a reduced amount of HCP.

64. (canceled)

65. The method of claim 63, wherein:
the loading buffer has a conductivity of 9 mS/cm to 11 mS/cm;
the loading buffer has a conductivity of 10 mS/cm or less, or 9 mS/cm or less; or
wherein the loading buffer has a conductivity of about 9 mS/cm, 9.5 mS/cm, 10 mS/cm, 10.5 mS/cm, or 11 mS/cm.

66-68. (canceled)

69. The method of claim 63, wherein the HCP is a Chinese Hamster Ovary (CHO) cell protein.

70-71. (canceled)

72. The method of claim 63, further comprising contacting the anion exchange resin with a wash buffer.

73. The method of claim 72, wherein:
the wash buffer has a conductivity of less than 11 mS/cm;
the wash buffer has a conductivity of 9 mS/cm to 11 mS/cm; or
the wash buffer has the same conductivity as the loading buffer.

74-75. (canceled)

76. The method of claim 72, wherein the loading buffer and/or the wash buffer comprises sodium chloride and/or sodium phosphate.

77. (canceled)

78. The method of claim 63, wherein:
the anion exchange resin is formatted as an anion exchange column or an anion exchange membrane; or
the anion exchange resin comprises a quaternary amine functional group.

79. (canceled)

80. The method of claim 63, wherein the sample containing vedolizumab and HCP is derived from a mammalian cell culture following one or more chromatographic separation steps.

81. The method of claim 80, wherein the one or more chromatographic separation steps comprise one or more steps selected from the group consisting of affinity chromatography, cation exchange chromatography, and ceramic hydroxyapatite (CHT) chromatography.

82. The method of claim 63, wherein:
the amount of HCP in the flow through material is 8 ppm or less, 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, or 2 ppm or less; or
the amount of HCP in the flow through material is reduced by at least 50% relative to the amount of HCP in the flow through material produced when the method is performed using the same sample with a loading buffer having a conductivity greater than 12 mS/cm.

83-84. (canceled)

85. The method of claim 63, wherein the method comprises incorporating the composition into a pharmaceutical formulation.

86. The method of claim 85, wherein the pharmaceutical formulation is a lyophilized pharmaceutical formulation or a liquid pharmaceutical formulation.

87-89. (canceled)

90. The method of claim 45, wherein the liquid pharmaceutical formulation is suitable for subcutaneous administration to a human.

91. A composition comprising vedolizumab produced by or is obtainable by the method of claim 63.

92. (canceled)

93. The composition of claim 91, wherein the amount of HCP in the composition is 8 ppm or less, 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, or 2 ppm or less.

94. A method of increasing the yield of vedolizumab recovered following elution from a mixed mode chromatography resin, comprising equilibrating the mixed mode chromatography resin with an equilibration buffer, loading a solution comprising vedolizumab and a loading buffer onto the mixed mode chromatography resin such that vedolizumab binds the mixed mode chromatography resin, washing the mixed mode chromatography resin with a wash buffer, and eluting vedolizumab from the mixed mode chromatography resin with an elution buffer, wherein the equilibration buffer, the loading buffer, and/or the wash buffer have a pH at or below 7.0.

95. The method of claim 94, wherein:
the equilibration buffer, the loading buffer, and/or the wash buffer have a pH of 6.0-7.0, a pH of 6.5-7.0, or a pH of 6.6-6.8; or
the equilibration buffer, the loading buffer, and/or the wash buffer have a salt concentration of 30 mM to 70 mM, 40 mM to 70 mM, 50 mM to 65 mM, or 55 mM-65 mM.

96-101. (canceled)

102. The method of claim 95, wherein the salt comprises sodium chloride and/or sodium phosphate.

103-108. (canceled)

109. The method of claim 94, wherein:
the equilibration buffer, the loading buffer, and/or the wash buffer have the same pH;
the equilibration buffer, the loading buffer, and/or the wash buffer have the same salt concentration; or
the equilibration buffer, the loading buffer, and/or the wash buffer are the same buffer.

110-111. (canceled)

112. The method of claim 94, wherein the mixed mode resin is a ceramic hydroxyapatite resin.

113. A composition comprising vedolizumab, wherein the composition is produced by or is obtainable by the method of claim 22.


SEQUENCE LISTING (10 sequences)

SEQ ID NO: 1 - protein, 121 aa, Artificial Sequence [sequence omitted]

SEQ ID NO: 2 - protein, 5 aa, Artificial Sequence
  Description of Artificial Sequence Synthetic peptide
SYWMH

SEQ ID NO: 3 - protein, 17 aa, Artificial Sequence
  Description of Artificial Sequence Synthetic peptide
EIDPSESNTNYNQKFKG

SEQ ID NO: 4 - protein, 12 aa, Artificial Sequence
  Description of Artificial Sequence Synthetic peptide
GGYDGWDYAIDY

SEQ ID NO: 5 - protein, 112 aa, Artificial Sequence [sequence omitted]

SEQ ID NO: 6 - protein, 16 aa, Artificial Sequence
  Description of Artificial Sequence Synthetic peptide
RSSQSLAKSYGNTYLS

SEQ ID NO: 7 - protein, 7 aa, Artificial Sequence
  Description of Artificial Sequence Synthetic peptide
GISNRFS

SEQ ID NO: 8 - protein, 9 aa, Artificial Sequence
  Description of Artificial Sequence Synthetic peptide
LQGTHQPYT

SEQ ID NO: 9 - protein, 451 aa, Artificial Sequence
  Description of Artificial Sequence Synthetic polypeptide
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  Description of Artificial Sequence Synthetic polypeptide
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC